(19) United States
(12) Patent Application Publication
(10) Pub. No.: US 2017/0218228 A1
(43) Pub. Date: Aug. 3, 2017

(54) Three Dimensional Printing of Bio-Ink Compositions

(71) Applicant: Tufts University, Medford, MA (US)

(72) Inventors: Rodrigo R. JOSE, Medford, MA (US); Fiorenzo OMENETTO, Lexington, MA (US); David KAPLAN, Concord, MA (US)

(73) Assignee: Tufts University, Medford, MA (US)

(21) Appl. No.: 15/329,419

(22) PCT Filed: Jul. 30, 2015
(86) PCT No.: PCT/US15/42764

§ 371 Date: Jan. 26, 2017

Related U.S. Application Data
(60) Provisional application No. 62/030,903, filed on Jul. 30, 2014.

(51) Int. Cl. C09D 189/00; A61L 31/14; A61L 31/16; A61L 31/18; B29C 67/00; B33Y 30/00; B33Y 10/00; B33Y 80/00; C09D 7/12; A61L 31/04; B33Y 70/00

(52) U.S. Cl. CPC C09D 189/00; A61L 31/047; A61L 31/148; A61L 31/16; A61L 31/18; B33Y 70/00; B33Y 30/00; B33Y 10/00; B33Y 80/00; C09D 7/1216; B29C 67/0059; B29C 67/0085; B29K 2995/0056; B29L 2031/7532

(57) ABSTRACT

3D printing of biopolymer-based inks provides for manufacturing a broad range of products with desirable properties. A print nozzle may be charged to form a cone-shaped ink droplet to result in increased resolution, more reliable contact with irregular surfaces, and a mechanism to control contacting the ink to the print surface.

### RELATED APPLICATIONS

This international patent application claims the benefit of priority under 35 U.S.C. 119(e) of U.S. provisional patent application No. 62/030,903, filed Jul. 30, 2014, entitled “THREE DIMENSIONAL PRINTING OF BIO-INK COMPOSITIONS”, the contents of which is hereby incorporated by reference in its entirety herein.

### GOVERNMENT SUPPORT

This invention was made with government support under grant no. 3P41EB002520-0951, awarded by the National Institutes of Health. The United States Government has certain rights in the invention.

### BACKGROUND

Three-dimensional printing is a type of computer-based printing that creates a three-dimensional object by progressively depositing material onto a substrate (i.e., a printable surface). The concept of three-dimensional printing has been around for over thirty years, but availability of the technology has been limited commercially until the last several years. In many current three-dimensional printing systems, an ink-jet-type printer is used to serially print a material such as a thermoplastic, a metal alloy, or a plaster as layers of particles or three-dimensional dots on the substrate. Computer-control of the location and number of such layers can direct so-called “additive manufacturing” of a designed article.

### SUMMARY

The present invention encompasses a recognition that certain biological compositions are particularly suitable for use as inks in printing technologies (e.g., ink jet and/or 3D printing technologies), and can be valuably employed to generate biocompatible three dimensional (“3D”) structures with surprising and beneficial attributes (e.g., structural and/or physical properties).

The present invention provides, among other things, certain biologically-based ink compositions (herein, “bio-ink compositions”), as well as articles and/or devices that are engineered and fabricated from such compositions. In certain embodiments, provided bio-ink compositions are self-curing. In certain embodiments, provided bio-ink compositions are substantially free of organic solvent. In some embodiments, provided bio-ink compositions are characterized in that, upon printing, they cure to form a crystallized layer that is substantially insoluble in water so that the crystallized layers do not dissolve, denature, and/or decompose when exposed to subsequent printed layers. Thus, in many embodiments, provided bio-ink compositions display material and/or chemical features that are suitable for use as 3D-printable inks.

Implementations of the invention are useful for a wide range of applications, including but not limited to: medical/surgical devices, imaging, optoelectronics, photonics, therapeutics, biomedical and tissue engineering, synthetic biology, drug delivery, and a variety of consumer products. The present invention also provides methods of preparing bio-ink compositions, methods of printing, and improved printing apparatus.

In some embodiments, bio-ink compositions for use in accordance with the present invention are printed, extruded, and/or deposited on a surface. In accordance with some embodiments, micro-scale, nano-scale, and pico-scale level printed structures are fabricated on the surface of a printable substrate from certain bio-ink compositions disclosed herein.

In some embodiments, when printed, extruded, and/or deposited bio-ink compositions form crystallized layers. In some embodiments, crystallized layers of bio-ink compositions are defined by a repeating secondary structure, such as an alpha-helix or a beta-sheet and/or hydrogen bonding.

In some embodiments, such printed structures include two-dimensional (“2D”) structures.

In some embodiments, bio-ink compositions are characterized in that when formed, resultant crystallized layers are substantially insoluble. In some embodiments, substantially insoluble layers do not dissolve, degrade, denature, and/or decompose when exposed to solvents or additional printed layers. In some embodiments, substantially insoluble layers do not dissolve, degrade, denature, and/or decompose once transferred physiological environments, simulated physiological environments, or completely submersed in solvent, for example water/phosphate buffered saline (PBS).

In some embodiments, 3D structures form when layers of bio-ink compositions ink are printed, extruded, and/or deposited atop previous layers. In some embodiments, printable bio-ink compositions for use in accordance with the present invention form 3D structures when individual layers are serially printed, extruded, and/or deposited on a printable substrate and without a need to machine, mill, or mold patterns in solid materials to form such 3D structures.

In some embodiments, bio-ink compositions for use in accordance with the present invention self-cure. In some embodiments, bio-ink compositions that self-cure do not require damaging cure mechanisms yet produce robust structures. In some embodiments, bio-ink compositions substantially concurrently self-cure upon printing, extruding, and/or depositing on a printable surface. In some embodiments, a short drying and/or curing time occurs after printing, extruding, and/or depositing of a bio-ink composition. In some embodiments, a short drying and/or curing time occurs between printing of subsequent layers. In some embodiments, a short drying and/or curing time is in a range between about 0.1 seconds and about 600 seconds. In some embodiments, drying time is dependent on a layer thickness. In some embodiments, drying time is dependent on a volume of ink. In some embodiments, drying time is dependent on environmental factors. In some embodiments, environmental factors include, for example, temperature and/or humidity.

In some embodiments, bio-ink compositions for use in accordance with the present invention that are printed, extruded, and/or deposited generate 3D structures that possess more consistent geometry and more regular features, including sharp angles and clean edges. In some embodiments, 3D structures formed from bio-ink compositions for use in accordance with the present invention have consistent geometry and/or more regular features that are more easily achievable and can be maintained during exposure to subsequent printings, solvents, and/or physiological environments.

In some embodiments, bio-ink compositions are characterized in that when formed, resultant crystallized layers are partially soluble when exposed to solvents or additional printed layers. In some embodiments, partially soluble layers dissolve, degrade, denature, and/or decompose over a predetermined time and/or a shortened time relative to a substantially insoluble crystallized layer.

In some embodiments, bio-ink compositions include a polypeptide. In some embodiments, polypeptides and fragments thereof may be used to make bio-ink compositions as described herein. Suitable polypeptides for practicing the present invention may be produced from various sources, for examples, including: regenerated (e.g., purified) proteins from natural sources, recombinant proteins or co-polymers produced in heterologous systems, synthetic or chemically produced peptides, or any combination of these sources.

In some embodiments, polypeptides (e.g., families and subfamilies of such proteins) suitable for carrying out the present invention include the following: fibroins, actins, collagens, catenins, claudins, coilins, elastins, elaunins, extensins, fibrillins, lamins, laminins, keratins, tublins, viral structural proteins, zein proteins (seed storage protein) and any combinations thereof.

In some embodiments, polypeptides for use in accordance with present invention are or comprise silk (e.g., silk fibroin).

In some embodiments, described bio-ink compositions comprise a polypeptide having a specified range or ranges of molecular weights (e.g., fragments). In some embodiments, described bio-ink compositions are substantially free of protein fragments exceeding a specified molecular weight. Where such fragments correspond to reduced size, relative to the naturally occurring full-length counterpart, such polypeptide fragments are broadly herein referred to as “low molecular weight.” In some embodiments, bio-ink compositions are comprised of low molecular weight polypeptides, for example in that the bio-ink compositions are substantially free of, and/or are prepared from solutions that are substantially free of, polypeptides having a molecular weight above about 400 kDa. In some embodiments, described biopolymer inks are substantially free of protein fragments over 200 kDa. In some embodiments, the highest molecular weight polymers in provided bio-ink compositions are less than about 300 kDa-about 400 kDa (e.g., less than about 400 kDa, less than about 375 kDa, less than about 350 kDa, less than about 325 kDa, less than about 300 kDa, etc.). In some embodiments, provided bio-ink compositions are comprised of polymers (e.g., protein polymers) having molecular weights within the range of about 20 kDa-about 400 kDa.

In some embodiments, provided polypeptides have molecular weights within a range between a lower bound (e.g., about 20 kDa, about 30 kDa, about 40 kDa, about 50 kDa, about 60 kDa, or more) and an upper bound (e.g., about 400 kDa, about 375 kDa, about 350 kDa, about 325 kDa, about 300 kDa, or less). In some embodiments, bio-ink compositions for use in accordance with the present invention are fabricated from polypeptides having a molecular weight ranging between about 3.5 kDa and about 120 kDa. In some embodiments, such bio-ink compositions are particularly useful. Those skilled in the art will appreciate that, typically, when a polypeptide is said to include a specified molecular weight (including within a specified molecular weight range), the polypeptide is substantially free of other molecular weight species of that polypeptide.

Discussed herein and/or known in the art are various technologies for obtaining or preparing bio-polymers of particular molecular weights. To give but one example, it is known in the art that different molecular weight preparations of silk fibroin may be prepared or obtained by boiling silk solutions for different amounts of time. For example, established conditions (see, for example, L. S. Wray, et. al., 99 J. Biomedical Materials Research Part B: Applied Biomaterials, (2011), which is incorporated by reference in its entirety herein) are known to generate silk fibroin compositions with maximal molecular weights in the range of about 300 kD-about 400 kD after about 5 minutes of boiling; compositions with molecular weights about 60 kD are can be achieved under comparable conditions after about 60 minutes of boiling.

In some particular embodiments, bio-ink compositions for use in accordance with the present invention are provided, prepared, and/or manufactured from a solution of silk fibroin that has been boiled for at least about 5, 10, 15, 20, 25, 30, 35, 40, 45, 50, 55, 60, 70, 80, 90, 100, 110, 120, 150, 180, 210, 240, 270, 310, 340, 370, 410 minutes or more. In some embodiments, such boiling is performed at a temperature within the range of: about 30° C., about 35° C., about 40° C., about 45° C., about 50° C., about 55° C., about 60° C., about 65° C., about 70° C., about 75° C., about 80° C., about 85° C., about 90° C., about 95° C., about 100° C., about 105° C., about 110° C., about 115° C., about at least 120° C. In some embodiments, such boiling is performed at a temperature below about 65° C. In some embodiments, such boiling is performed at a temperature of about 60° C. or less.

In some embodiments, bio-ink compositions for use in accordance with the present invention are provided, prepared, and/or manufactured from a polypeptide (e.g., silk such as silk fibroin) solution of about 0.5 wt % polypeptide to about 30 wt % polypeptide. In some embodiments, bio-ink compositions for use in accordance with the present invention are provided, prepared, and/or manufactured from a polypeptide (e.g., silk, such as silk fibroin) solution that is less than about 30 wt % polypeptide. In some embodiments, bio-ink compositions for use in accordance with the present invention are provided, prepared, and/or manufactured from a polypeptide solution that is less than about 20 wt % polypeptide. In some embodiments, bio-ink compositions for use in accordance with the present invention are provided, prepared, and/or manufactured from a polypeptide solution that is less than about 10 wt % polypeptide. Indeed, in some embodiments, the present invention provides the surprising teaching that useful bio-ink compositions with particularly valuable properties can be provided, prepared, and/or manufactured from a polypeptide solution that is less than about 10 wt % polypeptide, or even that is about 5% wt %, about 4 wt %, about 3 wt %, about 2 wt %, about 1 wt % polypeptide or less.

In some embodiments, bio-ink compositions for use in accordance with the present invention are provided, prepared, and/or manufactured from a solution of polypeptide (e.g., of silk such as silk fibroin) that is adjusted to (e.g., by dialysis) and/or maintained at a sub-physiological pH (e.g., at or below a pH significantly under pH 7). In some embodiments, bio-ink compositions are provided, prepared, and/or manufactured from a solution of protein polymer with a pH for instance about 6 or less, about 5 or less, about 4 or less, about 3 or less, about 2 or less, about 1.5 or less, or about 1 or less. In some embodiments, bio-ink compositions are provided, prepared, and/or manufactured from a solution of protein polymer with a pH in a range for example of at least 6, at least 7, at least 8, at least 9, and at least about 10.

In some embodiments, bio-ink composition compositions include a humectant. A humectant is generally a water soluble solvent and any one of a group of hygroscopic substances with hydrating properties (i.e., used to keep things moist). A humectant's affinity to form hydrogen bonds with molecules of water confers some important crucial traits. Humectants often are a molecule with several hydrophilic groups, most often hydroxyl groups, however, amines and carboxyl groups, sometimes esterified, can be encountered as well.

In some embodiments, humectants suited for use in the present invention include the following, non-limiting examples: butylene glycol, hexylene glycol, glyceryl triacetate (E1518), neoagarobiose, propylene glycol (E1520), vinyl alcohol. In some embodiments, humectants are sugar alcohols and/or sugar polyols, for examples, including: aloe vera gel, alpha hydroxy acids (e.g., lactic acid), arabitol, ethylene glycol, erythritol, fucitol, galactitol, glycerol, glycerin, 1,2,6-hexanetriol, iditol, inositol, isomalt, lactitol, maltitol, maltitol (E965), maltotetraitol, maltotriitol, mannitol, MP Diol, polyglycitol, polymeric polyols (e.g., polydextrose (E1200)), quillaia (E999), 1,3-propanediol, ribitol, sorbitol, sorbitol (E420), threitol, urea, volemitol, xylitol, and any combinations thereof.

In some embodiments, a humectant for use in accordance with the present invention is or comprises glycerol.

In some embodiments, humectants for use in accordance with the present invention are provided from a solution of about 0.5 wt % humectant to about 30 wt % humectant (e.g., glycerol). In some embodiments, bio-ink compositions for use in accordance with the present invention are provided, prepared, and/or manufactured from a humectant solution that is less than about 10 wt % humectant. In some embodiments, the present invention provides bio-ink compositions provided, prepared, and/or manufactured from a humectant solution that is less than about 10 wt % humectant, or even that is about 5% wt %, about 4 wt %, about 3 wt %, about 2 wt %, about 1 wt % humectant or less.

In some embodiments, bio-ink compositions for use in accordance with the present invention are a composition prepared as a blend of a polypeptide and a humectant.

In some embodiments, bio-ink compositions including polypeptides and humectants form crystallized layers that are substantially insoluble when exposed to solvents and/or physiological conditions.

In some embodiments of the present invention, provided bio-ink compositions that include polypeptides and humectants, may be particularly useful for printing inks into insoluble crystallized layers upon which additional layers can be subsequently printed. In some embodiments, bio-ink compositions comprising polypeptides and humectants form crystallized layers immediately cured upon printing or substantially concurrent with a step of extruding, and/or depositing.

In some embodiments, a ratio of a polypeptide to a humectant modulates a degree of imparted crystallinity of an article and/or device printed from a bio-ink composition described herein. In some embodiments, bio-ink compositions comprising polypeptides and humectants form partially soluble crystallized layers that are characterized in that partially soluble crystallized layers dissolve, degrade, denature, and/or decompose over a predetermined time and/or a shortened time relative to a substantially insoluble crystallized layer.

In some embodiments, bio-ink compositions comprising polypeptides and humectants form crystallized layers whereby subsequent additional crystallized layers of the ink can be printed substantially concurrent atop prior layers to form a 3D structure.

Thus, in some aspects, the present disclosure provides the insight that a humectant as an additive in to a bio-ink compositions confers certain advantages to 3D-printing ink compositions (e.g., bio-ink compositions).

In some embodiments, bio-ink compositions for use in accordance with the present invention are a composition prepared as a blend of a polypeptide and a humectant, wherein the polypeptide comprises about 2% w/v to about 25% w/v of the bio-ink composition and the humectant comprises about 2% w/v to about 30% w/v of the bio-ink composition.

In some embodiments, bio-ink compositions for use in accordance with the present invention are a composition prepared as a blend of a polypeptide and a humectant, wherein the polypeptide comprises a range of about 0.05 mM to about 10 mM of the ink and the humectant comprises a range of about 5 mM to about 1000 mM of the ink. In some embodiments, bio-ink compositions for use in accordance with the present invention are a composition prepared as a blend of a polypeptide and a humectant, wherein the polypeptide comprises 0.5 mM of the ink and the humectant comprises about 400 mM of the ink.

A ratio of a polypeptide to a humectant may be about 20 to 1, about 15 to 1, about 10 to 1, about 5 to 1, about 2 to 1, or about 1 to 1.

In some embodiments, bio-ink compositions do not require organic solvents. In some embodiments, bio-ink compositions are substantially free of organic solvents.

In some embodiments, provided and/or utilized bio-ink compositions do not require drying steps such as, for example, alcohol treatments, shearing, gelling, or e-gelling in between printing, extruding, or depositing bio-ink compositions of the present invention. In some embodiments, multiple additional layers of bio-ink composition as disclosed herein may be immediately or substantially concurrently applied atop prior layers to form a 3D structure without such intervening steps.

In some embodiments, bio-ink compositions have a viscosity of between about 1-20 centipoise (cP) as measured at room temperature of between about 18-26° C.

Bio-ink compositions in accordance with the present invention may also contain one or more added agents, or additives, or dopants. In some embodiments, such added agents are stabilized by the polypeptide present in the ink composition.

In some embodiments, described bio-ink compositions comprise one or more suitable viscosity-modifying agents (i.e., viscosity modifiers or viscosity adjusters).

In some embodiments, bio-ink compositions may contain a surfactant, which acts as a wetting and/or penetrating agent.

In some embodiments, bio-ink compositions agents add functionality. In some embodiments, bio-ink compositions comprising a polypeptide and a humectant and do not utilize alcohol treatments, shearing, gelling, or e-gelling to cure. As such, in some embodiments, bio-ink compositions can incorporate biological agents such as drugs, growth factors, or cells without potential harm caused by such treatments. A non-limiting list of suitable agents that may be added to functionalize provided bio-ink compositions include: cells and fractions thereof (viruses and viral particles; prokaryotic cells such as bacteria; eukaryotic cells such as mammalian cells and plant cells; fungi), conductive particles, dyes/pigments, inorganic particles, metallic particles, proteins and fragments or complexes thereof (e.g., enzymes, antigens, antibodies and antigen-binding fragments thereof). In some embodiments, agents include, for example nucleic acids and/or nucleic acid analogues. In some embodiments, agents, for examples include: anti-proliferative, diagnostic agents, immunological agents, therapeutic agents, preventative agent, prophylactic agents, to name but a few are incorporated within bio-ink compositions of the present invention. In some embodiments, agents includes drugs (e.g., antibiotics, small molecules or low molecular weight organic compounds). In some embodiments, agents added to bio-ink compositions disclosed herein are releaseable. In some embodiments, a controlled release of an agent is achieved through diffusion as layers of ink dissolve, degrade, denature, decompose, and/or delaminate.

In some embodiments, provided bio-ink compositions are biocompatible. In some embodiments, provided bio-ink compositions are biodegradable. In some embodiments, provided bio-ink compositions are biocompatible and biodegradable.

In some embodiments, the present invention includes methods of printing bio-ink compositions as described herein. In some embodiments, methods utilizing such bio-ink compositions comprise a polypeptide and a humectant. In some embodiments, methods utilize bio-ink compositions comprising silk fibroin and glycerol.

In some embodiments, provided 3D-printing technologies include steps of applying stacked layers of a bio-ink composition to a surface (e.g., a substrate surface) to create a 3D structure.

In some embodiments, methods include flowing a bio-ink composition from a print head onto a substrate while moving the flowing ink and substrate relative to one another so that the ink is printed on a surface of a substrate.

In some embodiments, methods of the present invention include bio-ink compositions that do not require steps of curing and/or solvents to cure printed bio-ink compositions so that subsequent additional layers can be printed substantially concurrent atop after printing, extruding, and/or deposition of a prior layer of a bio-ink composition to form a 3D structure.

In some embodiments, provided 3D printing technologies therefore involve application of multiple layers of ink (e.g., bio-ink composition) without intervening drying steps such as alcohol treatments, shearing, gelling, e-gelling, or crystallization. Some such embodiments, therefore avoid a need for chemical treatments, evaporation and/or annealing periods, and/or electrogelation steps between layer applications.

Among other things, the present invention also provides a printer system also herein referred to as a 3D printer or an extruder for printing bio-ink compositions as described herein.

In some embodiments, a 3D printer system for use in accordance with the present invention may include a print head with at least one extruder configured to dispense components of a bio-ink composition during printing. In some embodiments, a 3D printing system includes a print head having at least one extruder configured to provide bio-ink composition onto a surface of a printable substrate. In some embodiments, at least one extruder includes more than 1, more than 2, more than 3, more than 4, more than 5, or more than 10 extruders.

In some embodiments, a 3D printer system as disclosed herein includes multi-motor stepper controlled robotics. In some embodiments, a 3D printing system includes a multi-motor stepper for high precision movement. In some embodiments, multi-motor steppers control movement of a substrate. In some embodiments, multi-motor steppers control movement of printer head. In some embodiments, multi-motor steppers control movement of a least one extruder. In some embodiments, such robotics are suited for precise control of movement of printing components so that printing, depositing, and/or extruding of bio-ink compositions is accomplished with high resolution and low volume. In some embodiments, low volume deposition provides for enhanced curing of bio-ink compositions.

In some embodiments, a 3D printing system includes a ground electrode and a power supply configured to apply a voltage between a least one extruder nozzle and a ground electrode to cause a bio-ink composition to form a Taylor cone as it exits an extruder nozzle. In some embodiments, a 3D printer for use in accordance with the present invention may further include a controller configured to control an applied voltage to selectably contact and disengage a Taylor cone from a surface in a predetermined manner in accordance with a programmed pattern. In some embodiments, a 3D printing system includes a power supply configured to apply a voltage between the at least one extruder nozzle and the ground electrode to cause the bio-ink composition to form a Taylor cone as it exits the extruder nozzle.

In some embodiments, methods of the present invention include applying a voltage to a bio-ink compositions while flowing from a print head. Applying a voltage in such a manner will cause disclosed bio-ink compositions to form a Taylor cone. In some embodiments, provided 3D-printing methods include steps of applying a voltage between a conductive extruder nozzle of a print head through which a bio-ink composition is printed and a ground electrode on a side of a substrate onto which the bio-ink composition is printed, which side is opposite the print head. In some embodiments, methods further comprise contacting a tip of a Taylor cone with a substrate. In some embodiments, methods include: applying a voltage while dragging a Taylor cone across a surface of a substrate, thereby printing an ink on a surface of a substrate along a path defined by movement. In some certain embodiments, methods of the present invention further include electrically controlling an applied voltage to selectably contact and disengage a Taylor cone from the surface. In some embodiments, an applied voltage, for example, is at least about 0.25 kV, is at least about 0.5 kV, at least about 1 kV, at least about 1.5 kV, at least about 2 kV, at least about 2.5 kV, at least about 3 kV, at least about 3.5 kV, at least about 4 kV, at least about 4.5 kV, at least about 5 kV, or combinations thereof wherein the voltage is fluctuated between and among any of these.

In some embodiments, a 3D printer system of the present invention includes a printable substrate. In some embodiments, a printable substrate is rotatable substrate. In some embodiments, a rotatable substrate is a tube.

In some embodiments, provided 3D-printing methods include steps of rotating a substrate onto which a 3D structure is being printed relative to a print head through which a bio-ink composition is printed via formation of a Taylor cone, while dragging a Taylor cone across a rotating substrate surface so that a tubular structure is formed. In some such embodiments, a substrate may be rotated about an axis that is perpendicular to a direction of bio-ink composition flow from a print head.

In some embodiments, of the present invention, 3D printing of a bio-ink composition is utilized to generate an article (e.g., an implantable article) comprising a coating, wherein the coating, and/or optionally the article, may be constructed by 3D bio-ink composition printing. In certain such embodiments, a bio-ink composition pattern may be configured to indicate a presence of a coating, e.g., applied onto some or all surfaces of the article. In some such embodiments, a coating may comprise one or more agents including for example one or more biologically active agents (e.g., drugs). In some such embodiments, an article may be implantable (e.g., configured and otherwise appropriate for implantation into a body).

In some embodiments, the present invention allows fused filament fabrication to be conducted similar to conventional thermoset 3D printing polymers, but without the side-effect of heat damage to a printed article. In some embodiments, the present invention further allows multi-layer fused filament fabrication to occur without intermittent steps which would damage sensitive incorporated molecules such as drugs, growth factors, or cells.

In some embodiments, bio-ink compositions for use in accordance with the present invention when cured are removable from a printable surface. In some embodiments, a silk-glycerol blend bio-ink composition printed onto a substrate is very flexible, yet robust. Thin prints, for example, on an order of about 5 μm to about 1500 μm can easily be removed (or peeled) from the substrate without breaking.

In some embodiments, of the present invention, 3D printing of a bio-ink composition may be utilized to generate an article having a device body and further having a bio-ink composition pattern comprised of markings, for example at respective ends of a device body, to allow for identification of the article and/or its location. In certain such embodiments, an article is implantable and/or markings permit detection of an article, for example via X-ray imaging. In certain particular embodiments, such articles may be detected and/or monitored for example during and/or after implantation in a body, e.g., via detection of bio-ink composition markings.

In some embodiments, a bio-ink composition as described herein with a radiopaque marker added printed onto a surface of a device body in a predetermined pattern is useful as identifiable via X-ray imaging when placed in situ in a patient in situ.

In some particular embodiments, provided 3D printing technologies are effectively utilized to produce an article such as a stent or an anastomosis device.

### BRIEF DESCRIPTION OF THE DRAWING

FIG. 1 shows a silk film being lifted from a substrate.

FIG. 2 shows a summary of the solubility of films produced from various silk/polyol blends.

FIG. 3 shows a complex shape formed from bio-ink composition blends.

FIG. 4 shows printed bio-ink composition droplets.

FIG. 5 shows printed bio-ink composition droplets.

FIG. 6 shows printed bio-ink composition droplets.

FIG. 7 shows printed bio-ink composition droplets.

FIG. 8 shows bio-ink composition printed on the outer diameter of tubing.

FIG. 9 shows a 3D bio-ink composition printing system.

FIG. 10 shows a 3D bio-ink composition printing system highlighting multiple extruders.

FIG. 11 shows an extruder with a standard droplet profile passing over an imperfect surface.

FIG. 12 shows an electrically charged extruder with a Taylor cone droplet profile passing over an imperfect surface.

FIG. 13 shows an extruder with a standard droplet profile and an electrically charged extruder with a droplet having the profile of a Taylor cone.

FIG. 14 show structures printed onto substrates using the bio-ink composition and a 3D bio-ink composition printing system.

FIG. 15 show structures printed onto substrates using the bio-ink composition and a 3D bio-ink composition printing system.

FIG. 16 shows profilometry data for three bio-ink composition depositions.

FIG. 17 shows a silk-glycerol bio-ink composition printed onto a substrate.

FIG. 18 shows printed radiopaque bio-ink composition patterns for degradable surgical implants.

FIG. 19 shows printed radiopaque bio-ink composition patterns for degradable surgical implants.

FIG. 20 shows resorbable radiopaque bio-ink composition markers printed onto a polymer implant substrate.

FIG. 21 shows a pattern of drug-containing bio-ink composition microdroplets.

FIG. 22 shows stress profiles of drug-containing bio-ink composition microdroplet patterns when exposed to fluid streams.

FIG. 23 shows stress profiles of drug-containing bio-ink composition microdroplet patterns when exposed to fluid streams.

FIG. 24 shows a pattern of drug-containing bio-ink composition microdroplets on a continuous substrate.

FIG. 25 shows a pattern of drug-containing bio-ink composition microdroplets on a perforated substrate.

FIG. 26 shows an interferometry analysis of the 3D surface profile of a bio-ink composition droplet and pattern of droplets.

FIG. 27 shows an interferometry analysis of the 3D surface profile of a bio-ink composition droplet and pattern of droplets.

FIG. 28 shows a substrate mounting system.

FIG. 29 shows a substrate mounting system.

FIG. 30 Process flow of device fabrication: a) Coating of rods for clip and coupler components; b) Spherical barb tip deposition for coupler components; c) Removal of tubes from rods for clip components; d) Removal of tubes with spherical barbs from rods for couplers; e) Initial trimming of coupler components; f) Initial trimming of clip components from tube, and creation of seats using biopsy punch.

FIG. 31 shows a Phase contrast images of (a) the coupler device barb and edge of the luminal opening; (b) high and low magnification of the hydrated coupler device cross-section showing layers of deposited silk:glycerol; (c and d) cross-section of dry and hydrated coupler devices.

FIG. 32 shows a) a schematic of a procedure for loading devices with heparin (top) and bulk loading via hydration in heparinized solution (bottom), b) a perfusion system used to perform release studies, c) a standard curve emission versus concentration, d) total quantity of Heparin released from example devices over a 24-hour time period, and e) amount of remnant drug retained in the devices at 0, 1, and 24 hours.

### DEFINITIONS

In order for the present disclosure to be more readily understood, certain terms are first defined below. Additional definitions for the following terms and other terms are set forth throughout the specification.

The present specification describes certain inventions relating to so-called “three-dimensional (3D) printing”, which can be distinguished from “two-dimensional (2D) printing” in that, the printed product has significant mass in three dimensions (i.e., has length, width, and height) and/or significant volume. By contrast, 2D printing generates printed products (e.g., droplets, sheets, layers) that, although rigorously three-dimensional in that they exist in three-dimensional space, are characterized in that one dimension is significantly small as compared with the other two. By analogy, those skilled in the art will appreciate that an article with dimensions of a piece of paper could reasonably be considered to be a “2D” article relative to a wooden block (e.g., a 2×4×2 block of wood), which would be considered a “3D” article. Those of ordinary skill will therefore readily appreciate the distinction between 2D printing and 3D printing, as those terms are used herein. In many embodiments, 3D printing is achieved through multiple applications of certain 2D printing technologies, having appropriate components and attributes as described herein.

In this application, unless otherwise clear from context, the term “a” may be understood to mean “at least one.” As used in this application, the term “or” may be understood to mean “and/or.” In this application, the terms “comprising” and “including” may be understood to encompass itemized components or steps whether presented by themselves or together with one or more additional components or steps. Unless otherwise stated, the terms “about” and “approximately” may be understood to permit standard variation as would be understood by those of ordinary skill in the art. Where ranges are provided herein, the endpoints are included. As used in this application, the term “comprise” and variations of the term, such as “comprising” and “comprises,” are not intended to exclude other additives, components, integers or steps.

As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Administration”: As used herein, the term “administration” refers to the administration of a composition to a subject. Administration may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal and vitreal.

“Associated”: As used herein, the term “associated” typically refers to two or more entities in physical proximity with one another, either directly or indirectly (e.g., via one or more additional entities that serve as a linking agent), to form a structure that is sufficiently stable so that the entities remain in physical proximity under relevant conditions, e.g., physiological conditions. In some embodiments, associated entities are covalently linked to one another. In some embodiments, associated entities are non-covalently linked. In some embodiments, associated entities are linked to one another by specific non-covalent interactions (i.e., by interactions between interacting ligands that discriminate between their interaction partner and other entities present in the context of use, such as, for example: streptavidiin/avidin interactions, antibody/antigen interactions, etc.). Alternatively or additionally, a sufficient number of weaker non-covalent interactions can provide sufficient stability for moieties to remain associated. Exemplary non-covalent interactions include, but are not limited to, affinity interactions, metal coordination, physical adsorption, host-guest interactions, hydrophobic interactions, pi stacking interactions, hydrogen bonding interactions, van der Waals interactions, magnetic interactions, electrostatic interactions, dipole-dipole interactions, etc.

“Biocompatible:” As used herein, the term “biocompatible” is intended to describe any material which does not elicit a substantial detrimental response in vivo.

“Biodegradable”: As used herein, the term “biodegradable” is used to refer to materials that, when introduced into cells, are broken down by cellular machinery (e.g., enzymatic degradation) or by hydrolysis into components that cells can either reuse or dispose of without significant toxic effect(s) on the cells. In certain embodiments, components generated by breakdown of a biodegradable material do not induce inflammation and/or other adverse effects in vivo. In some embodiments, biodegradable materials are enzymatically broken down. Alternatively or additionally, in some embodiments, biodegradable materials are broken down by hydrolysis. In some embodiments, biodegradable polymeric materials break down into their component and/or into fragments thereof (e.g., into monomeric or submonomeric species). In some embodiments, breakdown of biodegradable materials (including, for example, biodegradable polymeric materials) includes hydrolysis of ester bonds. In some embodiments, breakdown of materials (including, for example, biodegradable polymeric materials) includes cleavage of urethane linkages. Exemplary biodegradable polymers include, for example, polymers of hydroxy acids such as lactic acid and glycolic acid, including but not limited to poly(hydroxyl acids), poly(lactic acid)(PLA), poly(glycolic acid)(PGA), poly(lactic-co-glycolic acid)(PLGA), and copolymers with PEG, polyanhydrides, poly(ortho)esters, polyesters, polyurethanes, poly(butyric acid), poly(valeric acid), poly(caprolactone), poly(hydroxyalkanoates, poly(lactide-co-caprolactone), blends and copolymers thereof. Many naturally occurring polymers are also biodegradable, including, for example, proteins such as albumin, collagen, gelatin and prolamines, for example, zein, and polysaccharides such as alginate, cellulose derivatives and polyhydroxyalkanoates, for example, polyhydroxybutyrate blends and copolymers thereof. Those of ordinary skill in the art will appreciate or be able to determine when such polymers are biocompatible and/or biodegradable derivatives thereof (e.g., related to a parent polymer by substantially identical structure that differs only in substitution or addition of particular chemical groups as is known in the art).

As used herein, the phrase “characteristic sequence element” refers to a sequence element found in a polymer (e.g., in a polypeptide or nucleic acid) that represents a characteristic portion of that polymer. In some embodiments, presence of a characteristic sequence element correlates with presence or level of a particular activity or property of the polymer. In some embodiments, presence (or absence) of a characteristic sequence element defines a particular polymer as a member (or not a member) of a particular family or group of such polymers. A characteristic sequence element typically comprises at least two monomers (e.g., amino acids or nucleotides). In some embodiments, a characteristic sequence element includes at least 2, 3, 4, 5, 6, 7, 8, 9, 10, 11, 12, 13, 14, 15, 20, 25, 30, 35, 40, 45, 50, or more monomers (e.g., contiguously linked monomers). In some embodiments, a characteristic sequence element includes at least first and second stretches of continguous monomers spaced apart by one or more spacer regions whose length may or may not vary across polymers that share the sequence element.

“Comparable”: As used herein, the term “comparable”, as used herein, refers to two or more agents, entities, situations, sets of conditions, etc. that may not be identical to one another but that are sufficiently similar to permit comparison therebetween so that conclusions may reasonably be drawn based on differences or similarities observed. Those of ordinary skill in the art will understand, in context, what degree of identity is required in any given circumstance for two or more such agents, entities, situations, sets of conditions, etc. to be considered comparable.

“Conjugated”: As used herein, the terms “conjugated,” “linked,” “attached,” and “associated with,” when used with respect to two or more moieties, means that the moieties are physically associated or connected with one another, either directly or via one or more additional moieties that serves as a linking agent, to form a structure that is sufficiently stable so that the moieties remain physically associated under the conditions in which structure is used. Typically the moieties are attached either by one or more covalent bonds or by a mechanism that involves specific binding. Alternately, a sufficient number of weaker interactions can provide sufficient stability for moieties to remain physically associated.

“Dosage form”: As used herein, the term “dosage form” refers to a physically discrete unit of a therapeutic agent for administration to a subject. Each unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen).

“Hydrophilic”: As used herein, the term “hydrophilic” and/or “polar” refers to a tendency to mix with, or dissolve easily in, water.

“Hydrophobic”: As used herein, the term “hydrophobic” and/or “non-polar”, refers to a tendency to repel, not combine with, or an inability to dissolve easily in, water.

“Hygroscopic”: As used herein, the term “hygroscopic”

“Hydrolytically degradable”: As used herein, the term “hydrolytically degradable” is used to refer to materials that degrade by hydrolytic cleavage. In some embodiments, hydrolytically degradable materials degrade in water. In some embodiments, hydrolytically degradable materials degrade in water in the absence of any other agents or materials. In some embodiments, hydrolytically degradable materials degrade completely by hydrolytic cleavage, e.g., in water. By contrast, the term “non-hydrolytically degradable” typically refers to materials that do not fully degrade by hydrolytic cleavage and/or in the presence of water (e.g., in the sole presence of water).

As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

The phrase “non-natural amino acid” refers to an entity having the chemical structure of an amino acid (i.e.:

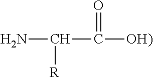

and therefore being capable of participating in at least two peptide bonds, but having an R group that differs from those found in nature. In some embodiments, non-natural amino acids may also have a second R group rather than a hydrogen, and/or may have one or more other substitutions on the amino or carboxylic acid moieties.

“Nucleic acid”: As used herein, the term “nucleic acid” as used herein, refers to a polymer of nucleotides. In some embodiments, a nucleic acid agent can be or comprise deoxyribonucleic acid (DNA), ribonucleic acid (RNA), peptide nucleic acid (PNA), morpholino nucleic acid, locked nucleic acid (LNA), glycol nucleic acid (GNA) and/or threose nucleic acid (TNA). In some embodiments, nucleic acid agents are or contain DNA; in some embodiments, nucleic acid agents are or contain RNA. In some embodiments, nucleic acid agents include naturally-occurring nucleotides (e.g., adenosine, thymidine, guanosine, cytidine, uridine, deoxyadenosine, deoxythymidine, deoxyguanosine, and deoxycytidine). Alternatively or additionally, in some embodiments, nucleic acid agents include non-naturally-occurring nucleotides including, but not limited to, nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups. In some embodiments, nucleic acid agents include phosphodiester backbone linkages; alternatively or additionally, in some embodiments, nucleic acid agents include one or more non-phosphodiester backbone linkages such as, for example, phosphorothioates and 5′-N-phosphoramidite linkages. In some embodiments, a nucleic acid agent is an oligonucleotide in that it is relatively short (e.g., less that about 5000, 4000, 3000, 2000, 1000, 900, 800, 700, 600, 500, 450, 400, 350, 300, 250, 200, 150, 100, 90, 80, 70, 60, 50, 45, 40, 35, 30, 25, 20, 15, 10 or fewer nucleotides in length).

“Physiological conditions”: As used herein, the phrase “physiological conditions” relates to the range of chemical (e.g., pH, ionic strength) and biochemical (e.g., enzyme concentrations) conditions likely to be encountered in the intracellular and extracellular fluids of tissues. For most tissues, the physiological pH ranges from about 6.8 to about 8.0 and a temperature range of about 20-40 degrees Celsius, about 25-40 degrees Celsius, about 30-40 degrees Celsius, about 35-40 degrees Celsius, about 37 degrees Celsius, atmospheric pressure of about 1. In some embodiments, physiological conditions utilize or include an aqueous environment (e.g., water, saline, Ringers solution, or other buffered solution); in some such embodiments, the aqueous environment is or comprises a phosphate buffered solution (e.g., phosphate-buffered saline).

The term “polypeptide”, as used herein, generally has its art-recognized meaning of a polymer of at least three amino acids, linked to one another by peptide bonds. In some embodiments, the term is used to refer to specific functional classes of polypeptides. For each such class, the present specification provides several examples of amino acid sequences of known exemplary polypeptides within the class; in some embodiments, such known polypeptides are reference polypeptides for the class. In such embodiments, the term “polypeptide” refers to any member of the class that shows significant sequence homology or identity with a relevant reference polypeptide. In many embodiments, such member also shares significant activity with the reference polypeptide. Alternatively or additionally, in many embodiments, such member also shares a particular characteristic sequence element with the reference polypeptide (and/or with other polypeptides within the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (i.e., a conserved region that may in some embodiments may be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a useful polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide. In some embodiments, a polypeptide may comprise natural amino acids, non-natural amino acids, or both. In some embodiments, a polypeptide may comprise only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups, e.g., modifying or attached to one or more amino acid side chains, and/or at the polypeptide's N-terminus, the polypeptide's C-terminus, or both. In some embodiments, a polypeptide may be cyclic. In some embodiments, a polypeptide is not cyclic. In some embodiments, a polypeptide is linear.

“Small molecule”: As used herein, the term “small molecule” is used to refer to molecules, whether naturally-occurring or artificially created (e.g., via chemical synthesis), having a relatively low molecular weight and being an organic and/or inorganic compound. Typically, a “small molecule” is monomeric and have a molecular weight of less than about 1500 g/mol. In general, a “small molecule” is a molecule that is less than about 5 kilodaltons in size. In some embodiments, a small molecule is less than about 4 kilodaltons, 3 kilodaltons, about 2 kilodaltons, or about 1 kilodalton. In some embodiments, the small molecule is less than about 800 daltons, about 600 daltons, about 500 daltons, about 400 daltons, about 300 daltons, about 200 daltons, or about 100 daltons. In some embodiments, a small molecule is less than about 2000 grams/mol, less than about 1500 grams/mol, less than about 1000 grams/mol, less than about 800 grams/mol, or less than about 500 grams/mol. In some embodiments, a small molecule is not a polymer. In some embodiments, a small molecule does not include a polymeric moiety. In some embodiments, a small molecule is not a protein or polypeptide (e.g., is not an oligopeptide or peptide). In some embodiments, a small molecule is not a polynucleotide (e.g., is not an oligonucleotide). In some embodiments, a small molecule is not a polysaccharide. In some embodiments, a small molecule does not comprise a polysaccharide (e.g., is not a glycoprotein, proteoglycan, glycolipid, etc.). In some embodiments, a small molecule is not a lipid. In some embodiments, a small molecule is a modulating agent. In some embodiments, a small molecule is biologically active. In some embodiments, a small molecule is detectable (e.g., comprises at least one detectable moiety). In some embodiments, a small molecule is a therapeutic. Preferred small molecules are biologically active in that they produce a local or systemic effect in animals, preferably mammals, more preferably humans. In certain preferred embodiments, the small molecule is a drug. Preferably, though not necessarily, the drug is one that has already been deemed safe and effective for use by the appropriate governmental agency or body. For example, drugs for human use listed by the FDA under 21 C.F.R. §§330.5, 331 through 361, and 440 through 460; drugs for veterinary use listed by the FDA under 21 C.F.R. §§500 through 589, incorporated herein by reference, are all considered acceptable for use in accordance with the present application.

“Stable”: As used herein, the term “stable,” when applied to compositions means that the compositions maintain one or more aspects of their physical structure and/or activity over a period of time under a designated set of conditions. In some embodiments, the period of time is at least about one hour; in some embodiments, the period of time is about 5 hours, about 10 hours, about one (1) day, about one (1) week, about two (2) weeks, about one (1) month, about two (2) months, about three (3) months, about four (4) months, about five (5) months, about six (6) months, about eight (8) months, about ten (10) months, about twelve (12) months, about twenty-four (24) months, about thirty-six (36) months, or longer. In some embodiments, the period of time is within the range of about one (1) day to about twenty-four (24) months, about two (2) weeks to about twelve (12) months, about two (2) months to about five (5) months, etc. In some embodiments, the designated conditions are ambient conditions (e.g., at room temperature and ambient pressure). In some embodiments, the designated conditions are physiologic conditions (e.g., in vivo or at about 37 degrees Celsius for example in serum or in phosphate buffered saline). In some embodiments, the designated conditions are under cold storage (e.g., at or below about 4 degrees Celsius, −20 degrees Celsius, or −70 degrees Celsius). In some embodiments, the designated conditions are in the dark.

“Substantially”: As used herein, the term “substantially”, and grammatical equivalents, refer to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result.

“Substantially free” As used herein, the term “substantially free” means that it is absent or present at a concentration below detection measured by a selected art-accepted means, or otherwise is present at a level that those skilled in the art would consider to be negligible in the relevant context.

“Sustained release”: As used herein, the term “sustained release” and in accordance with its art-understood meaning of release that occurs over an extended period of time. The extended period of time can be at least about 3 days, about 5 days, about 7 days, about 10 days, about 15 days, about 30 days, about 1 month, about 2 months, about 3 months, about 6 months, or even about 1 year. In some embodiments, sustained release is substantially burst-free. In some embodiments, sustained release involves steady release over the extended period of time, so that the rate of release does not vary over the extended period of time more than about 5%, about 10%, about 15%, about 20%, about 30%, about 40% or about 50%.

“Treating”: As used herein, the term “treating” refers to partially or completely alleviating, ameliorating, relieving, inhibiting, preventing (for at least a period of time), delaying onset of, reducing severity of, reducing frequency of and/or reducing incidence of one or more symptoms or features of a particular disease, disorder, and/or condition. In some embodiments, treatment may be administered to a subject who does not exhibit symptoms, signs, or characteristics of a disease and/or exhibits only early symptoms, signs, and/or characteristics of the disease, for example for the purpose of decreasing the risk of developing pathology associated with the disease. In some embodiments, treatment may be administered after development of one or more symptoms, signs, and/or characteristics of the disease.

As used herein, the term “variant” refers to an entity that shows significant structural identity with a reference entity but differs structurally from the reference entity in the presence or level of one or more chemical moieties as compared with the reference entity. In many embodiments, a variant also differs functionally from its reference entity. In general, whether a particular entity is properly considered to be a “variant” of a reference entity is based on its degree of structural identity with the reference entity. As will be appreciated by those skilled in the art, any biological or chemical reference entity has certain characteristic structural elements. A variant, by definition, is a distinct chemical entity that shares one or more such characteristic structural elements. To give but a few examples, a small molecule may have a characteristic core structural element (e.g., a macrocycle core) and/or one or more characteristic pendent moieties so that a variant of the small molecule is one that shares the core structural element and the characteristic pendent moieties but differs in other pendent moieties and/or in types of bonds present (single vs double, E vs Z, etc.) within the core, a polypeptide may have a characteristic sequence element comprised of a plurality of amino acids having designated positions relative to one another in linear or three-dimensional space and/or contributing to a particular biological function, a nucleic acid may have a characteristic sequence element comprised of a plurality of nucleotide residues having designated positions relative to on another in linear or three-dimensional space. For example, a variant polypeptide may differ from a reference polypeptide as a result of one or more differences in amino acid sequence and/or one or more differences in chemical moieties (e.g., carbohydrates, lipids, etc.) covalently attached to the polypeptide backbone. In some embodiments, a variant polypeptide shows an overall sequence identity with a reference polypeptide that is at least 85%, 86%, 87%, 88%, 89%, 90%, 91%, 92%, 93%, 94%, 95%, 96%, 97%, or 99%. Alternatively or additionally, in some embodiments, a variant polypeptide does not share at least one characteristic sequence element with a reference polypeptide. In some embodiments, the reference polypeptide has one or more biological activities. In some embodiments, a variant polypeptide shares one or more of the biological activities of the reference polypeptide. In some embodiments, a variant polypeptide lacks one or more of the biological activities of the reference polypeptide. In some embodiments, a variant polypeptide shows a reduced level of one or more biological activities as compared with the reference polypeptide. In many embodiments, a polypeptide of interest is considered to be a “variant” of a parent or reference polypeptide if the polypeptide of interest has an amino acid sequence that is identical to that of the parent but for a small number of sequence alterations at particular positions. Typically, fewer than 20%, 15%, 10%, 9%, 8%, 7%, 6%, 5%, 4%, 3%, 2% of the residues in the variant are substituted as compared with the parent. In some embodiments, a variant has 10, 9, 8, 7, 6, 5, 4, 3, 2, or 1 substituted residue as compared with a parent. Often, a variant has a very small number (e.g., fewer than 5, 4, 3, 2, or 1) number of substituted functional residues (i.e., residues that participate in a particular biological activity). Furthermore, a variant typically has not more than 5, 4, 3, 2, or 1 additions or deletions, and often has no additions or deletions, as compared with the parent. Moreover, any additions or deletions are typically fewer than about 25, about 20, about 19, about 18, about 17, about 16, about 15, about 14, about 13, about 10, about 9, about 8, about 7, about 6, and commonly are fewer than about 5, about 4, about 3, or about 2 residues. In some embodiments, the parent or reference polypeptide is one found in nature. As will be understood by those of ordinary skill in the art, a plurality of variants of a particular polypeptide of interest may commonly be found in nature, particularly when the polypeptide of interest is an infectious agent polypeptide.

### DETAILED DESCRIPTION OF CERTAIN EMBODIMENTS

The present disclosure describes bio-ink compositions and their use in various printing applications, including for example ink-jet printing and/or 3D-printing. Provided bio-ink compositions are particularly useful in biological contexts, for example to produce scaffolds useful for tissue engineering.

### Bio-ink Compositions

As described herein, the present invention provides bio-ink compositions suitable for use in printing applications (e.g., ink-jet printing and/or 3D printing).

Typically, a provided bio-ink composition is a liquid composition comprising a biologically-compatible polymer and a solvent or dispersing medium. In many embodiments, the composition is substantially free of organic solvents. In some embodiments, the composition is an aqueous composition (e.g., the solvent or dispersing medium is or comprises water). In some embodiments, a solvent and/or dispersing medium, for example, is or comprises water, cell culture medium, buffers (e.g., phosphate buffered saline), buffered solutions (e.g. PBS), polyols (for example, glycerol, propylene glycol, liquid polyethylene glycol, and the like), agar, gelatin, Dulbecco's Modified Eagle Medium, fetal bovine serum, or suitable combinations and/or mixtures thereof. Particularly desirable bio-ink compositions for use in the practice of the present invention are characterized by a viscosity of between about 1 centipoise (cP), and about 10,000 cP, where 1 cP=1 mPa-s=0.001 Pa s, as measured at a temperature of between about 10-50° C.

Among other things, the present invention encompasses the recognition that particularly useful bio-ink compositions are characterized in that, when deposited on a substrate, they can be cured to a form that is resistant to degradation by subsequent depositions. For example, in some embodiments, where a bio-ink composition is or comprises an aqueous composition, it is characterized in that it cures to a water-insoluble form, which can then be a substrate for deposition of a subsequent layer of the bio-ink without being significantly solubilized.

In some embodiments, appropriate bio-ink compositions for use in accordance with the present invention are self-curing (e.g., form layers that immediately cure upon printing, extruding, and/or depositing. In some embodiments, curing of appropriate bio-ink compositions is triggered by a curing agent (e.g., a chemical, electrophysical, and/or environmental agent, condition, or set thereof). In some embodiments, curing involves evaporation of some or all of a particular solvent. In some embodiments, curing involves structural modification (e.g., introduction of cross-links) to one or more components of a bio-ink composition, and particularly to a biopolymer component. In some embodiments, curing involves alteration in structural form of one or more components of a bio-ink composition, and particularly of a biopolymer component; in some such embodiments, curing involves a significant increase in crystallinity of a bio-ink composition and/or of a biopolymer component therein. In some embodiments, such an increase in crystallinity is attributable in whole or in part to an increase in beta-sheet character in a bio-ink composition and/or in a biopolymer component thereof, particularly in a polypeptide component thereof (e.g., in a silk fibroin polypeptide component thereof, as described in further detail below).

In some embodiments, curing involves conversion of a deposited bio-ink composition into a form that, as noted above, is resistant to degradation by application of subsequent printed layers. In some embodiments, curing involves conversion to a form that is characterized in that dissolves, degrades, denatures, and/or decomposes over a predetermined time and/or under predetermined conditions.

Those of ordinary skill will appreciate that desirably such curing occurs within a relatively short period of time after deposition, for example, between about 0.5 seconds and about 600 seconds. Additionally, in some embodiments, in contrast to competitive direct-write applications where silk has been printed directly into organic solvent (such as methanol) baths, a bio-ink composition is characterized in that it can be cured via evaporation-induced buckling of silk depositions which, when blended with certain non-toxic additives, cure to crystallized structural prints. In some embodiment, evaporation-induced buckling of silk depositions bypasses deleterious curing mechanisms.

On the other hand, particularly preferred bio-ink compositions are characterized that they can be maintained in an uncured state (e.g., in a liquid state, in some embodiments characterized by flowability as described herein). In some embodiments, bio-ink compositions are characterized that they can be maintained in an uncured state for a time sufficient to permit printing of at least one layer.

In some embodiments, bio-ink compositions remain in an uncured state for an extended period in a container for storage. In some embodiment, a storage container is a cartridge configured for a printing or extruding apparatus. In some embodiments, a cartridge may hold at least 1 mL, at least 5 mL, at least 10 mL, at least 15 mL, at least 20 mL, at least 50 mL, at least 100 mL or more. In some embodiments, a storage container is a drum, for example a 50 gallon drum. In some embodiments, a storage container may serve as a reservoir. In some embodiments, a storage container may include a pump line. In some embodiments, storage conditions include, for example, sealed in a glass container as 4° C. In some embodiments, storage conditions include, for example, sealed in a plastic container at room temperature. In some embodiments, storage conditions include, for example, a humidity in a range of less than about 1% to about 100%.

In some embodiments, storage of a silk solution may occur at a temperature of: about 1° C., about 2° C., about 3° C., about 4° C., about 5° C., about 6° C., about 7° C., about 8° C., about 9° C., about 10° C., about 15° C., about 20° C., about 25° C., about 30° C., about 35° C., about 40° C., about 45° C., or about at least 50° C.

Alternatively or additionally, in some embodiments, particularly desirable bio-ink compositions are characterized in that they can adopt or be converted to a stable-storage form. In some embodiments, bio-ink compositions may be stored for an extended period. In some embodiments, bio-ink compositions may be stored for at least a year, at least two years, at least five years, or more. In some embodiments, after extended storage, bio-ink compositions are equivalently printable.

In some embodiments, a bio-ink composition for use in the present invention are stored and/or utilized at a sub-physiological pH (e.g., at or below a pH significantly under pH 7). In some embodiments, provided compositions are prepared, manufactured, provided and/or maintained from a solution with a pH for instance about 6 or less, about 5 or less, about 4 or less, about 3 or less, about 2 or less, about 1.5 or less, or about 1 or less. In some embodiments, the pH is in a range for example of at least 6, at least 7, at least 8, at least 9, and at least about 10.

In some embodiments, bio-ink compositions for use in accordance with the present invention comprise, in addition to a biopolymer, a humectant and/or one or more other components or additives. In some embodiments, appropriate bio-ink compositions are substantially free of biologically incompatible or deleterious materials. In some embodiments, bio-ink compositions for use in the practice of the present invention comprise one or more other agents and/or functional moieties, for example, viscosity-modifying agents, surfactants, therapeutics, preventatives, diagnostics, pigments/dyes, and combinations thereof.

In some embodiments, bio-ink compositions for use in accordance with the present invention are characterized by their usefulness in a variety of printing applications that do not involve biologically incompatible or deleterious methodologies (e.g., heat treatments, contact with biologically incompatible agents, components, or conditions.

The present invention encompasses the recognition that certain provided compositions have characteristics particularly useful in 3D printing applications. The present invention particularly encompasses the recognition that certain bio-ink compositions developed for 2D printing may be utilized and/or adapted as described herein to achieve 3D printing, if developed, prepared and/or utilized to have appropriate characteristics and/or behavior as described herein; particular such bio-ink compositions of interest include those described in PCT Patent Application No. PCT/US2013/072435, filed on Nov. 27, 2013, the entire contents of which are hereby incorporated by reference.

Biologically-Compatible Polymers

As described herein, provided bio-ink compositions utilize a biocompatible polymer as the ink. In some embodiments, the biocompatible polymer is, comprises, or is a fragment or variant of, a biological polymer (i.e., a polymer that exists in nature). In some embodiments, a biocompatible polymer is or comprises a biodegradable polymer.

In general, a biocompatible polymer for use in accordance with the present invention may be obtained or provided using any available technology or source. For example, in some embodiments, a biocompatible polymer may be obtained from a natural source. In some embodiments, a biocompatible polymer may be obtained from a man-made source (e.g., a genetically engineered cell or organism, or a synthetic setting).

In many embodiments, a biocompatible polymer for use in accordance with the present invention is or comprises a polypeptide. In some embodiments, a polypeptide appropriate for use in the practice of the present invention is, comprises, or is a fragment or variant of a biological polypeptide. Useful such biological peptides include those selected from the group consisting of fibroins, actins, collagens, catenins, claudins, coilins, elastins, elaunins, extensins, fibrillins, keratins, lamins, laminins, silks, tublins, viral structural proteins, zein proteins (seed storage protein) and any combinations thereof.

In some embodiments, a polypeptide appropriate for use in a bio-ink composition as described herein shows significant sequence identity with a naturally-occurring reference polypeptide, or with another known reference polypeptide. In some embodiments, such a polypeptide may show at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% overall amino acid sequence identity with an appropriate reference polypeptide. Alternatively or additionally, in some embodiments, a polypeptide appropriate for use in a bio-ink composition as described herein shares at least one characteristic sequence element with such a reference polypeptide.

Silks

In some embodiments, a polypeptide is or comprises a silk polypeptide, such as a silk fibroin polypeptide. In nature, silk is produced as protein fiber, typically made by specialized glands of animals, and often used in nest construction. Organisms that produce silk include the Hymenoptera (bees, wasps, and ants and other types of arthropods, most notably various arachnids such as spiders (e.g., spider silk), also produce silk. Silk fibers generated by insects and spiders represent the strongest natural fibers known and rival even synthetic high performance fibers.

The first reported examples of silk being used as a textile date to ancient China (see Elisseeff, “The Silk Roads: Highways of Culture and Commerce,” Berghahn Books/UNESCO, New York (2000); see also Vainker, “Chinese Silk: A Cultural History,” Rutgers University Press, Piscataway, N.J. (2004)); it has been highly prized in that industry ever since. Indeed, silk has been extensively investigated for its potential in textile, biomedical, photonic and electronic applications. Glossy and smooth, silk is favored by not only fashion designers but also tissue engineers because it is mechanically tough but degrades harmlessly inside the body, offering new opportunities as a highly robust and biocompatible material substrate (see Altman et al., Biomaterials, 24: 401 (2003); see also Sashina et al., Russ. J. Appl. Chem., 79: 869 (2006)). Thus, even among biocompatible polymers (and particularly among biocompatible polypeptides, including natural polypeptides), silk and silk polypeptides are of particular interest and utility.

Silk fibroin is a polypeptide, like collagen, but with a unique feature: it is produced from the extrusion of an amino-acidic solution by a living complex organism (while collagen is produced in the extracellular space by self-assembly of cell-produced monomers). Silk is naturally produced by various species, including, without limitation: Antheraea mylitta; Antheraea pernyi; Antheraea yamamai; Galleria mellonella; Bombyx mori; Bombyx mandarina; Galleria mellonella; Nephila clavipes; Nephila senegalensis; Gasteracantha mammosa; Argiope aurantia; Araneus diadematus; Latrodectus geometricus; Araneus bicentenarius; Tetragnatha versicolor; Araneus ventricosus; Dolomedes tenebrosus; Euagrus chisoseus; Plectreurys tristis; Argiope trifasciata; and Nephila madagascariensis. Embodiments of the present invention may utilize silk proteins from any such organism. In some embodiments, the present invention utilizes silk or silk proteins from a silkworm, such as Bombyx mori (e.g., from cocoons or glands thereof). In some embodiments, the present invention utilizes silks or silk proteins from a spider, such as Nephila clavipes (e.g., from nests/webs or glands thereof).

In general, silk polypeptides for use in accordance with the present invention may be or include natural silk polypeptides, or fragments or variants thereof. In some embodiments, such silk polypeptides may be utilized as natural silk, or may be prepared from natural silk or from organisms that produce it. Alternatively, silk polypeptides utilized in the present invention may be prepared through an artificial process, for example, involving genetic engineering of cells or organisms (e.g., genetically engineered bacteria, yeast, mammalian cells, non-human organisms, including animals, or transgenic plants) to produce a silk polypeptide, and/or by chemical synthesis.

In some particular embodiments, silk polypeptides are obtained from cocoons produced by a silkworm, in certain embodiments by the silkworm Bombyx mori; such cocoons are of particular interest as a source of silk polypeptide because they offer low-cost, bulk-scale production of silk polypeptides. Moreover, isolation methodologies have been developed that permit preparation of cocoon silk, and particularly of Bombyx mori cocoon silk in a variety of forms suitable for various commercial applications.

Silkworm cocoon silk contains two structural proteins, the fibroin heavy chain (˜350 kDa) and the fibroin light chain (˜25 kDa), which are associated with a family of non-structural proteins termed sericins, that glue the fibroin chains together in forming the cocoon. The heavy and light fibroin chains are linked by a disulfide bond at the C-terminus of the two subunits (see Takei, et al. J. Cell Biol., 105: 175, 1987; see also Tanaka, et al J. Biochem. 114: 1, 1993; Tanaka, et al Biochim. Biophys. Acta., 1432: 92, 1999; Kikuchi, et al Gene, 110: 151, 1992). The sericins are a high molecular weight, soluble glycoprotein constituent of silk which gives the stickiness to the material. These glycoproteins are hydrophilic and can be easily removed from cocoons by boiling in water. This process is often referred to as “degumming” In some embodiments, silk polypeptide compositions utilized in accordance with the present invention are substantially free of sericins (e.g., contain no detectable sericin or contain sericin at a level that one of ordinary skill in the pertinent art will consider negligible for a particular use).

To give but one particular example, in some embodiments, silk polypeptide compositions for use in accordance with the present invention are prepared by processing cocoons spun by silkworm, Bombyx mori so that sericins are removed and silk polypeptides are solubilized. In some such embodiments, cocoons are boiled (e.g., for a specified length of time, often approximately 30 minutes) in an aqueous solution (e.g., of 0.02 M Na₂CO₃), then rinsed thoroughly with water to extract the glue-like sericin proteins. Extracted silk is then dissolved in a solvent, for example, LiBr (such as 9.3 M). A resulting silk fibroin solution can then be further processed for a variety of applications as described elsewhere herein.

In some embodiments, silk polypeptide compositions for use in the practice of the present invention comprise silk fibroin heavy chain polypeptides and/or silk fibroin light chain polypeptides; in some such embodiments, such compositions are substantially free of any other polypeptide. In some embodiments that utilize both a silk fibroin heavy chain polypeptide and a silk fibroin light chain polypeptide, the heavy and light chain polypeptides are linked to one another via at least one disulfide bond. In some embodiments, where the silk fibroin heavy and light chain polypeptides are present, they are linked via one, two, three or more disulfide bonds.

Exemplary natural silk polypeptides that may be useful in accordance with the present invention may be found in International Patent Publication Number WO 2011/130335, International Patent Publication Number WO 97/08315 and/or U.S. Pat. No. 5,245,012, the entire contents of each of which are incorporated herein by reference. Table 1, below, provides an exemplary list of silk-producing species and silk proteins:

TABLE 1
An exemplary list of silk-producing species and silk proteins (adopted from Bini et al. (2003), J.
Mol. Biol. 335(1): 27-40).

| | | | |
|---|---|---|---|
| Accession | Species | Producing gland | Protein |

Silkworms

| | | | |
|---|---|---|---|
| AAN28165 | Antheraea mylitta | Salivary | Fibroin |
| AAC32606 | Antheraea pernyi | Salivary | Fibroin |
| AAK83145 | Antheraea yamamai | Salivary | Fibroin |
| AAG10393 | Galleria mellonella | Salivary | Heavy-chain fibroin |
| | | | (N-terminal) |
| AAG10394 | Galleria mellonella | Salivary | Heavy-chain fibroin |
| | | | (C-terminal) |
| P05790 | Bombyx mori | Salivary | Fibroin heavy chain precursor, Fib-H, |
| | | | H-fibroin |
| CAA27612 | Bombyx mandarina | Salivary | Fibroin |
| Q26427 | Galleria mellonellla | Salivary | Fibroin light chain precursor, Fib-L, |
| | | | L-fibroin, PG-1 |
| P21828 | Bombyx mori | Salivary | Fibroin light chain precursor, Fib-L, |
| | | | L-fibroin |

Spiders

| | | | |
|---|---|---|---|
| P19837 | Nephila clavipes | Major ampullate | Spidroin 1, |
| | | | dragline silk fibroin 1 |
| P46804 | Nephila clavipes | Major ampullate | Spidroin 2, |
| | | | dragline silk fibroin 2 |
| AAK30609 | Nephila senegalensis | Major ampullate | Spidroin 2 |
| AAK30601 | Gasteracantha | Major ampullate | Spidroin 2 |
| | mammosa | | |
| AAK30592 | Argiope aurantia | Major ampullate | Spidroin 2 |
| AAC47011 | Araneus diadematus | Major ampullate | Fibroin-4, ADF-4 |
| AAK30604 | Latrodectus | Major ampullate | Spidroin 2 |
| | geometricus | | |
| AAC04503 | Araneus bicentenarius | Major ampullate | Spidroin 2 |
| AAK30615 | Tetragnatha versicolor | Major ampullate | Spidroin 1 |
| AAN85280 | Araneus ventricosus | Major ampullate | Dragline silk protein-1 |
| AAN85281 | Araneus ventricosus | Major ampullate | Dragline silk protein-2 |
| AAC14589 | Nephila clavipes | Minor ampullate | MiSp1 silk protein |
| AAK30598 | Dolomedes tenebrosus | Ampullate | Fibroin 1 |
| AAK30599 | Dolomedes tenebrosus | Ampullate | Fibroin 2 |
| AAK30600 | Tuagrus chisoseus | Combined | Fibroin 1 |
| AAK30610 | Plectreurys tristis | Larger ampule- | Fibroin 1 |
| | | shaped | |
| AAK30611 | Plectreurys tristis | Larger ampule- | Fibroin 2 |
| | | shaped | |
| AAK30612 | Plectreurys tristis | Larger ampule- | Fibroin 3 |
| | | shaped | |
| AAK30613 | Plectreurys tristis | Larger ampule- | Fibroin 4 |
| | | shaped | |
| AAK30593 | Argiope trifasciata | Flagelliform | Silk protein |
| AAF36091 | Nephila | Flagelliform | Fibroin, silk protein |
| | madagascariensis | | (N-terminal) |
| AAF36092 | Nephila | Flagelliform | Silk protein |
| | madagascariensis | | (C-terminal) |
| AAC38846 | Nephila clavipes | Flagelliform | Fibroin, silk protein |
| | | | (N-terminal) |
| AAC38847 | Nephila clavipes | Flagelliform | Silk protein |
| | | | (C-terminal) |

Silk fibroin polypeptides are characterized by a structure that typically reflects a modular arrangement of large hydrophobic blocks staggered by hydrophilic, acidic spacers, and, typically, flanked by shorter (˜100 amino acid), often highly conserved, terminal domains (at one or both of the N and C termini) In many embodiments, the hydrophobic blocks comprise or consist of alanine and/or glycine residues; in some embodiments alternating glycine and alanine; in some embodiments alanine alone. In many embodiments, the hydrophilic spacers comprise or consist of amino acids with bulky side-groups. Naturally occurring silk fibroin polypeptides often have high molecular weight (200 to 350 kDa or higher) with transcripts of 10,000 base pairs and higher and >3000 amino acids (reviewed in Omenetto and Kaplan (2010) Science 329: 528-531).

In some embodiments, core repeat sequences of the hydrophobic blocks found in silk fibroin polypeptides are represented by one or more of the following amino acid sequences and/or formulae:

(SEQ ID NO: 1)
(GAGAGS)5-15;
(SEQ ID NO: 2)
(GX)5-15 (X = V, I, A);
(SEQ ID NO: 3)
GAAS;
(SEQ ID NO: 4)
(S1-2A11-13);
(SEQ ID NO: 5)
GX1-4 GGX;
(SEQ ID NO: 6)
GGGX (X = A, S, Y, R, D V, W, R, D);
(SEQ ID NO: 7)
(S1-2A1-4)1-2;
(SEQ ID NO: 8)
GLGGLG;
(SEQ ID NO: 9)
GXGGXG (X = L, I, V, P);
(SEQ ID NO: 10)
GPX (X = L, Y, I); (GP(GGX)1-4 Y)n
(X = Y, V, S, A);
(SEQ ID NO: 11)
GRGGAn;
(SEQ ID NO: 12)
GGXn (X = A, T, V, S); GAG(A)6-7GGA;
and
(SEQ ID NO: 13)
GGX GX GXX (X = Q, Y, L, A, S, R).

In some embodiments, a fibroin polypeptide contains multiple hydrophobic blocks, e.g., 3, 4, 5, 6, 7, 8, 9, 10, 11, 12, 13, 14, 15, 16, 17, 18, 19 and 20 hydrophobic blocks within the polypeptide. In some embodiments, a fibroin polypeptide contains between 4-17 hydrophobic blocks. In some embodiments, a fibroin polypeptide comprises at least one hydrophilic spacer sequence (“hydrophilic block”) that is about 4-50 amino acids in length. Non-limiting examples of such hydrophilic spacer sequences include:

(SEQ ID NO: 14)
TGSSGFGPYVNGGYSG;
(SEQ ID NO: 15)
YEYAWSSE;
(SEQ ID NO: 16)
SDFGTGS;
(SEQ ID NO: 17)
RRAGYDR;
(SEQ ID NO: 18)
EVIVIDDR;
(SEQ ID NO: 19)
TTIIEDLDITIDGADGPI
and
(SEQ ID NO: 20)
TISEELTI.

In certain embodiments, a fibroin polypeptide contains a hydrophilic spacer sequence that is a variant of any one of the representative spacer sequences listed above. In some embodiments, a variant spacer sequence shows at least 75%, at least 80%, at least 85%, at least 90%, or at least 95% identity to one or more of the hydrophilic spacer sequences listed above, which may be considered to be reference hydrophilic spacer sequences.

In some embodiments, a fibroin polypeptide suitable for the present invention does not contain any of the hydrophilic spacer sequences listed above; in some embodiments, such a fibroin polypeptide further does not contain any variant of such a hydrophilic spacer sequence.

It is generally believed that features of silk fibroin polypeptide structure contribute to the material properties and/or functional attributes of the polypeptide. For example, sequence motifs such as poly-alanine (polyA) and polyalanine-glycine (poly-AG) are inclined to be beta-sheet-forming; the presence of one or more hydrophobic blocks as described herein therefore may contribute to a silk polypeptide's ability to adopt a beta-sheet conformation, and/or the conditions under which such beta-sheet adoption occurs.

In some embodiments, the silk fiber can be an unprocessed silk fiber, e.g., raw silk or raw silk fiber. The term “raw silk” or “raw silk fiber” refers to silk fiber that has not been treated to remove sericin, and thus encompasses, for example, silk fibers taken directly from a cocoon. Thus, by unprocessed silk fiber is meant silk fibroin, obtained directly from the silk gland. When silk fibroin, obtained directly from the silk gland, is allowed to dry, the structure is referred to as silk I in the solid state. Thus, an unprocessed silk fiber comprises silk fibroin mostly in the silk I conformation (a helix dominated structure). A regenerated or processed silk fiber on the other hand comprises silk fibroin having a substantial silk II (a (3-sheet dominated structure).

Inducing a conformational change in silk fibroin can facilitate formation of a solid-state silk fibroin and/or make the silk fibroin at least partially insoluble. Further, inducing formation of beta-sheet conformation structure in silk fibroin can prevent silk fibroin from contracting into a compact structure and/or forming an entanglement. In some embodiments, a conformational change in the silk fibroin can alter the crystallinity of the silk fibroin in the silk particles, such as increasing crystallinity of the silk fibroin, e.g., silk II beta-sheet crystallinity.

In some embodiments, the conformation of the silk fibroin in the silk fibroin foam can be altered after formation.

In some embodiments, bio-ink compositions as disclosed herein cure to possess some degree of silk II beta-sheet crystallinity.

In some embodiments, bio-ink compositions that cure form printed articles with a high degree of silk II beta-sheet crystallinity. In some embodiments, bio-ink compositions that subsequently form printed articles with a high degree of silk II beta-sheet crystallinity are insoluble to solvents. In some embodiments, bio-ink compositions that subsequently form printed articles with a high degree of silk II beta-sheet crystallinity are insoluble to immersion in solvents. In some embodiments, bio-ink compositions that subsequently form printed articles with a high degree of silk II beta-sheet crystallinity are insoluble when layers of a bio-ink composition are subsequently printed, deposited, and/or extruded atop a printed article.

In some embodiments, bio-ink compositions that cure form printed articles with a low degree of silk II beta-sheet crystallinity. In some embodiments, bio-ink compositions that subsequently form printed articles with a low degree of silk II beta-sheet crystallinity are at least partially soluble to solvents. In some embodiments, bio-ink compositions that subsequently form printed articles with a low degree of silk II beta-sheet crystallinity are at least partially soluble when layers of a bio-ink composition are subsequently printed, deposited, and/or extruded atop a printed article.

In some embodiments, physical properties of silk fibroin can be modulated when selecting and/or altering a degree of crystallinity of silk fibroin. In some physical properties of silk fibroin include, for example, mechanical strength, degradability, and/or solubility. In some embodiments, inducing a conformational change in silk fibroin can alter the rate of release of an active agent from the silk matrix.

In some embodiments, a conformational change can be induced by any methods known in the art, including, but not limited to, alcohol immersion (e.g., ethanol, methanol), water annealing, water vapor annealing, heat annealing, shear stress (e.g., by vortexing), ultrasound (e.g., by sonication), pH reduction (e.g., pH titration), and/or exposing the silk particles to an electric field and any combinations thereof.

Also, GXX motifs contribute to 31-helix formation; GXG motifs provide stiffness; and, GPGXX (SEQ ID NO: 22) contributes to beta-spiral formation. In light of these teachings and knowledge in the art (see, for example, review provided by Omenetto and Kaplan Science 329: 528, 2010), those of ordinary skill, reading the present specification, will appreciate the scope of silk fibroin polypeptides and variants thereof that may be useful in practice of particular embodiments of the present invention.

In some embodiments, bio-ink compositions as disclosed herein are or comprise a silk ionomeric composition. In some embodiments, bio-ink compositions as disclosed herein are or comprise ionomeric particles distributed in a solution. (See for example, WO 2011/109691 A2, to Kaplan et al., entitled Silk-Based Ionomeric Compositions, which describes silk-based ionomeric compositions and methods of manufacturing, which is hereby incorporated by reference in its entirety herein).

In some embodiments, bio-ink compositions comprising silk-based ionomeric particles may exist in fluid suspensions (or particulate solutions) or colloids, depending on the concentration of the silk fibroin. In some embodiments, bio-ink compositions comprising ionmeric particles include positively and negatively charged silk fibroin associated via electrostatic interaction.

In some embodiments, silk ionomeric particles are reversibly cross-linked through electrostatic interactions. In some embodiments, silk ionomeric compositions reversibly transform from one state to the other state when exposed to an environmental stimulus. In some embodiments, environmental stimuli silk ionomeric compositions respond to include for example, a change in pH, a change in ionic strength, a change in temperature, a change in an electrical current applied to the composition, or a change on a mechanical stress as applied to the composition. In some embodiments, silk ionomeric compositions transform into a dissociated charged silk fibroin solution.

Keratins

Keratins are members of a large family of fibrous structural proteins (see, for example, Moll et al, Cell 31:11 1982 that, for example, are found in the outer layer of human skin, and also provide a key structural component to hair and nails. Keratin monomers assemble into bundles to form intermediate filaments, which are tough and insoluble and form strong unmineralized tissues found in reptiles, birds, amphibians, and mammals.

Two distinct families of keratins, type I and type II, have been defined based on homologies to two different cloned human epidermal keratins (see Fuchs et al., Cell 17:573, 1979, which is hereby incorporated by reference in its entirety herein). Like other intermediate filament proteins, keratins contain a core structural domain (typically approximately 300 amino acids long) comprised of four segments in alpha-helical conformation separated by three relatively short linker segments predicted to be in beta-turn confirmation (see Hanukoglu & Fuchs Cell 33:915, 1983, which is hereby incorporated by reference in its entirety herein). Keratin monomers supercoil into a very stable, left-handed superhelical structure; in this form, keratin can multimerise into filaments. Keratin polypeptides typically contain several cysteine residues that can become crosslinked

In some embodiments, bio-ink compositions for use in the practice of the present invention comprise one or more keratin polypeptides.

Molecular Weight

The present disclosure appreciates that preparations of a particular biopolymer that differ in the molecular weight of the included biopolymer (e.g., average molecular weight and/or distribution of molecular weights) may show different properties relevant to practice of the present invention, including, for example, different viscosities and/or flow characteristics, different abilities to cure, etc. In some embodiments, a molecular weight of a biopolymer may impact a self-life of a bio-ink composition. Those of ordinary skill, reading the present disclosure and armed with knowledge in the art, will be able to prepare and utilize various bio-ink compositions with appropriate molecular weight characteristics for relevant embodiments of the invention.

In some particular embodiments, bio-ink compositions for use in accordance with the present invention include biopolymers whose molecular weight is within a range bounded by a lower limit and an upper limit, inclusive. In some embodiments, the lower limit is at least 1 kDa, at least 5 kDa, at least 10 kDa, at least 15 kDa, at least 20 kDa, at least 25 kDa, at least 30 kDa, at least 40 kDa, at least 50 kDa, at least 60 kDa, at least 70 kDa, at least 80 kDa, at least 90 kDa, at least 100 kDa, at least 150 kDa, at least 200 kDa; in some embodiments, the upper limit is less than 500 kDa, less than 450 kDa, less than 400 kDa, less than 350 kDa, less than 300 kDa, less than 250 kDa, less than 200 kDa, less than 175 kDa, less than 150 kDa, less than 120 kDa, less than 100 kDa, less than 90 kDa, less than 80 kDa, less than 70 kDa, less than 60 kDa, less than 50 kDa, less than 40 kDa, less than 30 kDa, less than 25 kDa, less than 20 kDa, less than 15 kDa, less than 12 kDa, less than 10 kDa, less than 9 kDa, less than 8 kDa, less than 7 kDa, less than 6 kDa, less than 5 kDa, less than 4 kDa, less than 3.5 kDa, less than 3 kDa, less than 2.5 kDa, less than 2 kDa, less than 1.5 kDa, or less than about 1.0 kDa, etc.

In some embodiments, a “low molecular weight” bio-ink composition is utilized. In some such embodiments, the composition contains biopolymers within a molecular weight range between about 3.5 kDa and about 120 kDa. To give but one example, low molecular weight silk fibroin compositions, and methods of preparing such compositions as may be useful in the context of the present invention, are described in detail in U.S. provisional application 61/883,732, entitled “LOW MOLECULAR WEIGHT SILK FIBROIN AND USES THEREOF,” the entire contents of which are incorporated herein by reference.

In some embodiments, bio-ink compositions for use in accordance with the present invention are substantially free of biopolymer components outside of a particular molecular weight range or threshold. For example, in some embodiments, a bio-ink composition is substantially free of biopolymer components having a molecular weight above about 400 kDa. In some embodiments, described biopolymer inks are substantially free of protein fragments over 200 kDa. “In some embodiments, the highest molecular weight biopolymers in provided bio-ink compositions have a molecular weight that is less than about 300 kDa-about 400 kDa (e.g., less than about 400 kDa, less than about 375 kDa, less than about 350 kDa, less than about 325 kDa, less than about 300 kDa, etc.).

In some embodiments, bio-ink compositions for use in accordance with the present invention are comprised of polymers (e.g., protein polymers) having molecular weights within the range of about 20 kDa-about 400 kDa, or within the range of about 3.5 kDa and about 120 kDa.

Those skilled in the art will appreciate that bio-ink compositions of a desired molecular weight (i.e., containing biopolymers within a particular molecular weight range and/or substantially free of biopolymers outside of that molecular weight range) may be prepared ab initio, or alternatively may be prepared either by fragmenting compositions of higher-molecular weight compositions, or by aggregating compositions of lower molecular weight polymers.

To give but one example, it is known in the art that different molecular weight preparations of silk fibroin polypeptides may be prepared or obtained by boiling silk solutions for different amounts of time. For example, established conditions (see, for example, Wray, et. al., 99 J. Biomedical Materials Research Part B: Applied Biomaterials 2011, which is hereby incorporated by reference in its entirety herein) are known to generate silk fibroin polypeptide compositions with maximal molecular weights in the range of about 300 kDa-about 400 kDa after about 5 minutes of boiling; compositions with molecular weights about 60 kDa are can be achieved under comparable conditions after about 60 minutes of boiling.

In some particular embodiments, silk fibroin polypeptide compositions of desirable molecular weight can be derived by degumming silk cocoons at or close to (e.g., within 5% of) an atmospheric boiling temperature, where such degumming involves at least about: 1 minute of boiling, 2 minutes of boiling, 3 minutes of boiling, 4 minutes of boiling, 5 minutes of boiling, 6 minutes of boiling, 7 minutes of boiling, 8 minutes of boiling, 9 minutes of boiling, 10 minutes of boiling, 11 minutes of boiling, 12 minutes of boiling, 13 minutes of boiling, 14 minutes of boiling, 15 minutes of boiling, 16 minutes of boiling, 17 minutes of boiling, 18 minutes of boiling, 19 minutes of boiling, 20 minutes of boiling, 25 minutes of boiling, 30 minutes of boiling, 35 minutes of boiling, 40 minutes of boiling, 45 minutes of boiling, 50 minutes of boiling, 55 minutes of boiling, 60 minutes or longer, including, e.g., at least 70 minutes, at least 80 minutes, at least 90 minutes, at least 100 minutes, at least 110 minutes, at least about 120 minutes or longer. As used herein, the term “atmospheric boiling temperature” refers to a temperature at which a liquid boils under atmospheric pressure.

In some embodiments, such degumming is performed at a temperature of: about 30° C., about 35° C., about 40° C., about 45° C., about 50° C., about 55° C., about 60° C., about 65° C., about 70° C., about 75° C., about 80° C., about 85° C., about 90° C., about 95° C., about 100° C., about 105° C., about 110° C., about 115° C., about 120° C., about 125° C., about 130° C., about 135° C., about 140° C., about 145° C., or about at least 150° C.

In some particular embodiments, bio-ink compositions for use in accordance with the present invention is provided, prepared, and/or manufactured from a solution of silk fibroin that has been boiled for at least about 5, 10, 15, 20, 25, 30, 35, 40, 45, 50, 55, 60, 70, 80, 90, 100, 110, 120, 150, 180, 210, 240, 270, 310, 340, 370, 410 minutes or more. In some embodiments, such boiling is performed at a temperature within the range of: about 30° C., about 35° C., about 40° C., about 45° C., about 50° C., about 55° C., about 60° C., about 65° C., about 70° C., about 75° C., about 80° C., about 85° C., about 90° C., about 95° C., about 100° C., about 105° C., about 110° C., about 115° C., about at least 120° C. In some embodiments, such boiling is performed at a temperature below about 65° C. In some embodiments, such boiling is performed at a temperature of about 60° C. or less.

In some embodiments, one or more processing steps of a bio-ink composition for use in accordance with the present invention is performed at an elevated temperature relative to ambient temperature. In some embodiments, such an elevated temperature can be achieved by application of pressure. For example, in some embodiments, elevated temperature (and/or other desirable effectis) can be achieved or simulated through application of pressure at a level between about 10-40 psi, e.g., at about 11 psi, about 12 psi, about 13 psi, about 14 psi, about 15 psi, about 16 psi, about 17 psi, about 18 psi, about 19 psi, about 20 psi, about 21 psi, about 22 psi, about 23 psi, about 24 psi, about 25 psi, about 26 psi, about 27 psi, about 28 psi, about 29 psi, about 30 psi, about 31 psi, about 32 psi, about 33 psi, about 34 psi, about 35 psi, about 36 psi, about 37 psi, about 38 psi, about 39 psi, or about 40 psi.

Concentration

In some embodiments, bio-ink compositions are prepared, provided, maintained and or utilized within a selected concentration range of biopolymer.

For example, in some embodiments, a bio-ink composition of interest may contain biopolymer (e.g., a polypeptide such as a silk fibroin polypeptide) at a concentration within the range of about 0.1 wt % to about 95 wt %, 0.1 wt % to about 75 wt %, or 0.1 wt % to about 50 wt %. In some embodiments, the aqueous silk fibroin solution can have silk fibroin at a concentration of about 0.1 wt % to about 10 wt %, about 0.1 wt % to about 5 wt %, about 0.1 wt % to about 2 wt %, or about 0.1 wt % to about 1 wt %. In some embodiments, the biopolymer is present at a concentration of about 10 wt % to about 50 wt %, about 20 wt % to about 50 wt %, about 25 wt % to about 50 wt %, or about 30 wt % to about 50 wt %. In some embodiments, a weight percent of silk in solution is about less than 1 wt %, is about less than 1.5 wt %, is about less than 2 wt %, is about less than 2.5 wt %, is about less than 3 wt %, is about less than 3.5 wt %, is about less than 4 wt %, is about less than 4.5 wt %, is about less than 5 wt %, is about less than 5.5 wt %, is about less than 6 wt %, is about less than 6.5 wt %, is about less than 7 wt %, is about less than 7.5 wt %, is about less than 8 wt %, is about less than 8.5 wt %, is about less than 9 wt %, is about less than 9.5 wt %, is about less than 10 wt %, is about less than 11 wt %, is about less than 12 wt %, is about less than 13 wt %, is about less than 14 wt %, is about less than 15 wt %, is about less than 16 wt %, is about less than 17 wt %, is about less than 18 wt %, is about less than 19 wt %, is about less than 20 wt %, is about less than 25 wt %, or is about less than 30 wt %.

In some particular embodiments, the present disclosure provides the surprising teaching that particularly useful bio-ink compositions with can be provided, prepared maintained and/or utilized with a biopolymer concentration that is less than about 10 wt %, or even that is about 5% wt %, about 4 wt %, about 3 wt %, about 2 wt %, about 1 wt % or less, particularly when the biopolymer is or comprises a silk biopolymer.

Humectants

In some embodiments, appropriate bio-ink compositions as described herein contain one or more humectants. In some embodiments, presence or level of included humectant impacts one or more cure characteristics of a bio-ink composition. For example, in some embodiments, presence or level of a humectant alters structure of a cured bio-ink composition and/or timing of curing under a given set of conditions. Alternatively or additionally, in some embodiments, presence or level of a humectant impacts conditions under which curing is achieved. In some particular examples, presence or level of a humectant may correlate with shortened cure times and/or reduced or eliminated need for external curing agents (e.g., chemical, electrophysical and/or environmental curing treatments). In some embodiments, presence or level of a humectant may correlate with increased flowability through a nozzle and/or reduced (frequency and/or degree of) nozzle clogging.

Generally, a humectant is a water soluble solvent and any one of a group of hygroscopic substances with hydrating properties, i.e., used to keep things moist. They often are a molecule with several hydrophilic groups, most often hydroxyl groups; however, amines and carboxyl groups, sometimes esterified, can be encountered as well (an ability to form hydrogen bonds with molecules of water, is typically a characteristic trait).

In some embodiments, humectants used in accordance with the present invention may be selected from a group consisting of, for example: propylene glycol (E1520), hexylene glycol, butylene glycol, glyceryl triacetate (E1518), vinyl alcohol, neoagarobiose, and combinations thereof. Alternatively or additionally, in some embodiments, bio-ink composition compositions comprise one or more humectants selected from the group consisting of sugar alcohols and sugar polyols. In some embodiments, sugar alcohols or sugar polyols for example include: alpha hydroxy acids (e.g., lactic acid), aloe vera gel, arabitol, erythritol, ethylene glycol, fucitol, galactitol, glycerol, glycerol/glycerin, honey, iditol, inositol, isomalt, lactitol, maltitol, maltitol (E965), maltotetraitol, maltotriitol, mannitol, MP Diol, polyglycitol, polymeric polyols (e.g., polydextrose (E1200)), quillaia (E999), ribitol, sorbitol (E420), threitol, urea, volemitol, xylitol, or combinations thereof. In some embodiments, a utilized humectant is or comprises glycerol. In some embodiments, a hutilized humectant is or comprises non-toxic polyols such as 1,3-propanediol and 1,2,6-hexanetriol.

In many embodiments, bio-ink compositions for use in the practice of the present invention are aqueous compositions that include a humectant (particularly such as glycerol and/or ethylene glycol).

In some embodiments, a bio-ink composition utilized in accordance with the present invention comprises humectant oat a level of about 0.5 wt % to about 30 wt %. In some embodiments, a bio-ink compositions for use in the practice of the present invention comprise less than about 10 wt % humectant. In some embodiments, a bio-ink composition for use in accordance with the present invention comprises less than about 10 wt % humectant, or even about 5% wt %, about 4 wt %, about 3 wt %, about 2 wt %, about 1 wt % humectant or less.

In some embodiments, a humectant for use in accordance with the present invention is or comprises glycerol. In some embodiments, a polypeptide is or comprises silk fibroin and glycerol is a humectant. In some embodiments, glycerol is incorporated as an additive specifically for the purpose of printing inks into insoluble crystallized layers upon which additional layers can be subsequently printed. Otherwise, subsequent print layers of fresh “ink” which may contain solvent, would dissolve the previous print layer, as they are printed. In some embodiments, bio-ink compositions for use in accordance with the present invention containing humectant do not need intermittent chemical treatments, lengthy evaporation, annealing periods, and/or electrogelation to cure. In some embodiments, bio-ink compositions comprising a biopolymer and a humectant form crystallized layers that immediately cure upon printing, extruding, and/or depositing and are therefore considered to be “self-curing”.

In some embodiments, bio-ink compositions for use in the practice of the present invention comprise a biopolymer ink (e.g., a polypeptide) and a humectant whereby a polypeptide and a humectant are present in absolute and relative amounts to one another so that the ink is characterized in that when printed on a substrate, it forms a crystallized layer whereby subsequent additional crystallized layers of an ink can be printed substantially concurrent atop prior layers to form a three-dimensional structure.

In some embodiments, a ratio of a polypeptide to a humectant modulates a degree of imparted crystallinity. In some embodiments, a ratio of a humectant (e.g. glycerol) to biopolymer (e.g., silk fibroin polypeptide) can be modulated to influence the degree of imparted crystallinity.

In some embodiments, a bio-ink composition for use in the practice of the present invention includes a biopolymer and a humectant in a biopolymer:humectant ratio that may be less than about 20 to 1, less than about 15 to 1, less than about 10 to 1, less than about 5 to 1, less than about 2 to 1, or less than about 1 to 1.

In some embodiments, inclusion of a humectant in a bio-ink composition for use in accordance with the present invention may materially improve one or more properties of the bio-ink composition relevant to printing inks into layers that cure to a form substantially resistant to degradation by subsequent printing of additional layers.

Agents/Additives

In some embodiments, bio-ink compositions for use in accordance with the present invention can further comprise one or more (e.g., one, two, three, four, five or more) agents, additives, and/or functional moieties. In some embodiments, an agent or additive may be covalently associated with a biopolymer or other component of a bio-ink composition (e.g., may be or comprise a functional moiety on such biopolymer or other component). In some embodiments, an agent or additive is not covalently associated with a biopolymer or other component of a bio-ink composition.

In some embodiments, an agent or additive is a component of a bio-ink composition in that it is combined with other components (e.g., biopolymer and/or humectant components). In some embodiments, an agent or additive is homogenously combined (e.g., mixed) with the other components. In some embodiments, an agent or additive is provided in a bio-ink composition so that it will be homogenously distributed within a printed layer of the bio-ink composition; in some embodiments, an agent or additive is provided in a bio-ink composition so that it will not be homogenously distributed within a printed layer of the bio-ink composition (e.g., will be present primarily on one surface or the other (or both) as compared with internally within the layer, will be present in a gradient throughout the layer, etc. In some embodiments, an agent or additive is provided in a bio-ink composition so that it will be released from a printed layer of the bio-ink composition, optionally according to a pre-determined rate and/or under a pre-determined set of conditions. In some embodiments, an agent or additive is incorporated after a bio-ink composition is printed (i.e. added to the printed article).

In some embodiments, an agent, additive, and/or functional moiety is or comprises a therapeutic agent, diagnostic agent, and/or preventative agent.

In general, an agent or additive can be present in a bio-ink composition as described herein at any desired amount. For example, in some embodiments, a total amount of agent or additives in an ink composition can be from about 0.01 wt % to about 99 wt %, from about 0.01 wt % to about 70 wt %, from about 5 wt % to about 60 wt %, from about 10 wt % to about 50 wt %, from about 15 wt % to about 45 wt %, or from about 20 wt % to about 40 wt %, of the total silk composition. In some embodiments, ratio of silk fibroin to additive in the composition can range from about 1000:1 (w/w) to about 1:1000 (w/w), from about 500:1 (w/w) to about 1:500 (w/w), from about 250:1 (w/w) to about 1:250 (w/w), from about 200:1 (w/w) to about 1:200 (w/w), from about 25:1 (w/w) to about 1:25 (w/w), from about 20:1 (w/w) to about 1:20 (w/w), from about 10:1 (w/w) to about 1:10 (w/w), or from about 5:1 (w/w) to about 1:5 (w/w).

In some embodiments, a bio-ink composition for use in accordance with the present invention may comprise a molar ratio of biopolymer to agent or additive of, e.g., at least 1000:1, at least 900:1, at least 800:1, at least 700:1, at least 600:1, at least 500:1, at least 400:1, at least 300:1, at least 200:1, at least 100:1, at least 90:1, at least 80:1, at least 70:1, at least 60:1, at least 50:1, at least 40:1, at least 30:1, at least 20:1, at least 10:1, at least 7:1, at least 5:1, at least 3:1, at least 1:1, at least 1:3, at least 1:5, at least 1:7, at least 1:10, at least 1:20, at least 1:30, at least 1:40, at least 1:50, at least 1:60, at least 1:70, at least 1:80, at least 1:90, at least 1:100, at least 1:200, at least 1:300, at least 1:400, at least 1:500, at least 600, at least 1:700, at least 1:800, at least 1:900, or at least 1:100.

In some embodiments, a bio-ink composition for use in accordance with the present invention may comprise a molar ratio of biopolymer to agent or additive of, e.g., at most 1000:1, at most 900:1, at most 800:1, at most 700:1, at most 600:1, at most 500:1, at most 400:1, at most 300:1, at most 200:1, 100:1, at most 90:1, at most 80:1, at most 70:1, at most 60:1, at most 50:1, at most 40:1, at most 30:1, at most 20:1, at most 10:1, at most 7:1, at most 5:1, at most 3:1, at most 1:1, at most 1:3, at most 1:5, at most 1:7, at most 1:10, at most 1:20, at most 1:30, at most 1:40, at most 1:50, at most 1:60, at most 1:70, at most 1:80, at most 1:90, at most 1:100, at most 1:200, at most 1:300, at most 1:400, at most 1:500, at most 1:600, at most 1:700, at most 1:800, at most 1:900, or at most 1:1000.

In some embodiments, a bio-ink composition for use in accordance with the present invention may comprise a molar ratio of biopolymer to agent or additive of, e.g. from about 1000:1 to about 1:1000, from about 900:1 to about 1:900, from about 800:1 to about 1:800, from about 700:1 to about 1:700, from about 600:1 to about 1:600, from about 500:1 to about 1:500, from about 400:1 to about 1:400, from about 300:1 to about 1:300, from about 200:1 to about 1:200, from about 100:1 to about 1:100, from about 90:1 to about 1:90, from about 80:1 to about 1:80, from about 70:1 to about 1:70, from about 60:1 to about 1:60, from about 50:1 to about 1:50, from about 40:1 to about 1:40, from about 30:1 to about 1:30, from about 20:1 to about 1:20, from about 10:1 to about 1:10, from about 7:1 to about 1:7, from about 5:1 to about 1:5, from about 3:1 to about 1:3, or about 1:1.

Viscosity-Modifying Agents

in some embodiments, bio-ink composition formulations useful in connection with the present invention may contain one or more viscosity-modifying agent, also referred to as viscosity modifiers or viscosity adjusters.

In some embodiments. an optimal range of viscosity is important for ensuing high quality, reproducible 3D printing. As such, in some embodiments, one or more of any suitable viscosity modifiers maybe used to adjust the viscosity of a bio-ink composition.

It should be noted, however, that in certain embodiments, bio-ink compositions as described herein not require addition of any such viscosity modifiers to be useful in the practice of the present invention. For example, so long as an ink composition viscosity is already at, near, or within a recommended viscosity or viscosity range as described herein, such addition may not be necessary.

In some embodiments, a humectant may function as a viscosity modifier, so that a bio-ink composition as described herein that includes a particular humectant (or level of such) may not require any additional viscosity-modifying agent.

In a broad sense, a viscosity modifying agent suitable for use in water-based inks is a water-soluble solvent that regulates or contributes to viscosity control in a liquid bio-ink composition. That, is, a viscosity modifying agent is one whose presence or level in a bio-ink composition as described herein.

In some embodiments, a bio-ink composition for use in the practice of the present invention contain between about 0.1-35 vol % of viscosity modifying agent.

In some embodiments, a bio-ink composition contains between about 0.5-30%, about 1.0-25%, about 5-20% of viscosity modifying agent (measured by volume). In some embodiments, s bio-ink composition contains about 0.5%, about 1.0%, about 2.0%, about 3.0%, about 4.0%, about 5.0%, about 6.0%, about 7.0%, about 8.0%, about 9.0%, about 10%, about 11%, about 12%, about 13%, about 14%, about 15%, about 16%, about 17%, about 18%, about 18%, about 20%, about 21%, about 22%, about 23%, about 24%, about 25%, about 26%, about 27%, about 28%, about 29%, about 30%, about 31%, about 32%, about 33%, about 34%, about 35%, of viscosity modifying agent (measured by volume).

Examples of other viscosity modifiers that may be included in bio-ink compositions utilized in accordance with the present invention may include, but are not limited to: acrylate esters, acrylic esters, acrylic monomer, aliphatic mono acrylate, aliphatic mono methacrylate, alkoxylated lauryl acrylate, alkoxylated phenol acrylate, alkoxylated tetrahydrofurfuryl acrylate, C12-C14 alkyl methacrylate, aromatic acrylate monomer, aromatic methacrylate monomer, caprolactone acrylate, cyclic trimethylol-propane formal acrylate, cycloaliphatic acrylate monomer, dicyclopentadienyl methacrylate, diethylene glycol methyl ether methacrylate, epoxidized soybean fatty acid esters, epoxidized linseed fatty acid esters, epoxy acrylate, epoxy (meth)acrylate, 2-(2-ethoxy-ethoxy) ethyl acrylate, ethoxylated (4) nonyl phenol acrylate, ethoxylated (4) nonyl phenol methacrylate, ethoxylated nonyl phenol acrylate, glucose, fructose, corn syrup, gum syrup, hydroxy-terminated epoxidized 1,3-polybutadiene, isobornyl acrylate, isobornyl methacrylate, isodecyl acrylate, isodecyl methacrylate, isooctyl acrylate, isooctyl methacrylate, lauryl acrylate, lauryl methacrylate, methoxy polyethylene glycol (350) monoacrylate, methoxy polyethylene glycol (350) monomethacrylate, methoxy poly-ethylene glycol (550) monoacrylate, methoxy polyethylene glycol (550) mono-methacrylate, nonyl-phenyl polyoxyethylene acrylate, octyldecyl acrylate, 2-phenoxyethyl acrylate, 2-phenoxyethyl methacrylate, polybutadiene polymer, polyester acrylate, polyester methacrylate, polyether acrylate, polyether methacrylate, polysorbates, stearyl acrylate, stearyl methacrylate, syrups, tetrahydrofurfuryl acrylate, tetrahydrofurfuryl methacrylate, triethylene glycol ethyl ether methacrylate, 3,3,5-trimethylcyclohexyl methacrylate, urethane acrylate and urethane methacrylate, and combinations thereof.

Surfactants

In some embodiments, bio-ink compositions for use in the practice of the present invention may contain a surfactant agent, for example which works as a wetting and/or penetrating agent. In some embodiments, addition of a surfactant agent to a bio-ink composition can modify or affect the surface tension of a the bio-ink composition (particularly of an aqueous bio-ink composition). In some embodiments, surface tension influences characteristics such as a bio-ink composition's flowability and or extrudability during printing.

In some embodiments, a surfactant agent is present at a concentration within a range between about 0.05-about 20%, e.g., between about 0.1-10% (either by volume or by weight) of a bio-ink composition.

Additives, Dopants, and Biologically Active Agents

In any of the embodiments, described herein, bio-ink compositions for use in the practice of the present invention may further include one or more agent(s) (e.g., dopants and additives) suitable for a particular intended purpose. In some embodiments, addition of such agents (or dopants) may be said to “functionalize” a bio-ink composition by providing added functionality.

Non-limiting examples of suitable agents (or dopants) to be added for functionalization of bio-ink compositions include but are not limited to: conductive or metallic particles; inorganic particles; dyes/pigments; drugs (e.g., antibiotics, small molecules or low molecular weight organic compounds); proteins and fragments or complexes thereof (e.g., enzymes, antigens, antibodies and antigen-binding fragments thereof); cells and fractions thereof (viruses and viral particles; prokaryotic cells such as bacteria; eukaryotic cells such as mammalian cells and plant cells; fungi); anti-proliferative agents, antibodies or fragments or portions thereof (e.g., paratopes or complementarity-determining regions), antibiotics or antimicrobial compounds, antigens or epitopes, aptamers, biopolymers, carbohydrates, cell attachment mediators (such as RGD), cytokines, cytotoxic agents, diagnostic agents (e.g. contrast agents; radionuclides; and fluorescent, luminescent, and magnetic moieties), drugs, enzymes, growth factors or recombinant growth factors and fragments and variants thereof, hormone antagonists, hormones, immunological agents, lipids, metals, nanoparticles (e.g., gold nanoparticles), nucleic acid analogs, nucleic acids (e.g., DNA, RNA, siRNA, modRNA, RNAi, and microRNA agents), nucleotides, nutraceutical agents, oligonucleotides, peptide nucleic acids (PNA), peptides, prodrugs, prophylactic agents (e.g. vaccines), proteins, radioactive elements and compounds, small molecules, therapeutic agents (e.g. antibiotics, NSAIDs, glaucoma medications, angiogenesis inhibitors, neuroprotective agents), toxins, or any combinations thereof

In some embodiments, bio-ink compositions for use in the practice of the present invention may further include inorganic fillers. In some embodiments, inorganic fillers provide structural support and strength to the material. In some embodiments, inorganic fillers provide support for incorporation of functionalized structures. In some embodiments, inorganic fillers include, for example, silica particles, hydroxyapatite particles, gold particles, or combinations thereof. Those skilled in the art will recognize that the fillers listed herein represent an exemplary, not comprehensive, list of inorganic filler materials and/or particles.

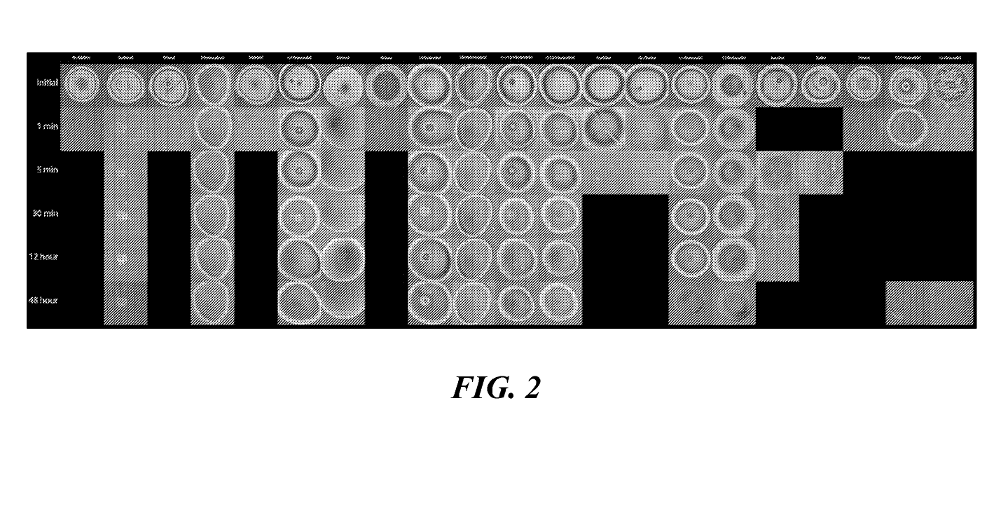

In some embodiments, printing anisotropically soluble layers may benefit from the inclusion of additional additives which impart various degrees of solubility to the film yet produce similar mechanical or hygroscopic properties. Such additions would minimize undesirable warping or stress localization between phases composing the printed layer to allow the production and handling of thin prints, such as illustrated in FIG. 2.

In some embodiments, printing, extruding or depositing bio-ink compositions for use in creating complex and/or hollow structures. In some embodiments, bio-ink compositions for use in creating complex and/or hollow structures include a pair of bio-ink compositions. In some embodiments, a pair of bio-ink compositions are useful for producing large scale, complex, irregular, and/or hollow 3D biocompatible, bioresorbable printing shapes. In some embodiments, a pair of bio-ink compositions include a sacrificial support material ink and permanent structural material ink. In some embodiments, a sacrificial support material ink dissolves leaving hollow structures behind.

In some embodiments, a bio-ink composition sacrificial support material ink further comprises an additive. In some embodiments, an additive suited for use in a sacrificial support material ink includes a hydrolyzed protein. In some embodiments, dissolvable inks contain linear polyols. In some embodiments, an additive suited for use in a sacrificial support material ink includes gelatin. In some embodiments, a bio based ink permanent structural material ink further comprises an additive. In some embodiments, an additive suited for use in a permanent structural material ink includes a polysaccharide. In some embodiments, an additive suited for use in a permanent structural material ink includes agar. In some embodiments, a specific pair for a process include a support material including 10% gelatin, 5% silk, 1% glycerol bulked bio-ink composition structural material is a 5% silk, 5% agar, 1% glycerol bulked bio-ink composition.

In some embodiments, a bio-ink composition may include additives for blending, for example, polyvinyl alcohol (PVA). In some embodiments, PVA solutions can also be used as a soluble support material.

In some embodiments, a useful additive for a bio-ink composition is a porogen. In some embodiments, porogens include poly(methyl methacrylate) (PMMA). In some embodiments, PMMA microspheres or rods can be included for porogenation.

In some embodiments, bio-ink compositions include a silk ionomeric composition as an additive or agent. (See for example, WO 2011/109691, which describes silk-based ionomeric compositions and methods of manufacturing, which is hereby incorporated by reference in its entirety herein).

In some embodiments, a useful additive is a biologically active agent. The term “biologically active agent” as used herein refers to any agent or entity which exerts at least one biological effect in vivo. For example, in some embodiments, a biologically active agent can be or comprise a therapeutic agent to treat or prevent a disease state or condition in a subject.

In certain embodiments, biologically active agents include, without limitation, organic molecules, inorganic materials, proteins, peptides, nucleic acids (e.g., genes, gene fragments, gene regulatory sequences, and antisense molecules), nucleoproteins, polysaccharides, glycoproteins, and lipoproteins. Classes of biologically active agents that can be incorporated into the composition described herein include, without limitation, anticancer agents, antibiotics, analgesics, anti-inflammatory agents, immunosuppressants, enzyme inhibitors, antihistamines, anti-convulsants, hormones, muscle relaxants, antispasmodics, ophthalmic agents, prostaglandins, anti-depressants, anti-psychotic substances, trophic factors, osteoinductive proteins, growth factors, and vaccines.

In some embodiments, a useful additive is or comprises a cell, e.g., a biological cell. Useful cells can come from any of a variety of sources, e.g., mammalian, insect, plant, etc. In some embodiments, the cell can be a human, rat or mouse cell. In general, any types of cells can be utilized. In may embodiments, cells are viable when present within a bio-ink composition, and/or within an article printed therewith. In some embodiments, cells that can be utilized in accordance with the present invention include, but are not limited to, mammalian cells (e.g. human cells, primate cells, mammalian cells, rodent cells, etc.), avian cells, fish cells, insect cells, plant cells, fungal cells, bacterial cells, and hybrid cells. In some embodiments, exemplary cells that can be can be utilized in accordance with the present invention include platelets, activated platelets, stem cells, totipotent cells, pluripotent cells, and/or embryonic stem cells. In some embodiments, exemplary cells include, but are not limited to, primary cells and/or cell lines from any tissue. For example, cardiomyocytes, myocytes, hepatocytes, keratinocytes, melanocytes, neurons, astrocytes, embryonic stem cells, adult stem cells, hematopoietic stem cells, hematopoietic cells (e.g. monocytes, neutrophils, macrophages, etc.), ameloblasts, fibroblasts, chondrocytes, osteoblasts, osteoclasts, neurons, sperm cells, egg cells, liver cells, epithelial cells from lung, epithelial cells from gut, epithelial cells from intestine, liver, epithelial cells from skin, etc, and/or hybrids thereof, can be included in the silk/platelet compositions disclosed herein. Those skilled in the art will recognize that the cells listed herein represent an exemplary, not comprehensive, list of cells. Cells can be obtained from donors (allogenic) or from recipients (autologous). Cells can be obtained, as a non-limiting example, by biopsy or other surgical means known to those skilled in the art.

In some embodiments, a utilized cell can be a genetically modified cell. For example, in some embodiments, a cell can be genetically modified to express and secrete a desired compound, e.g. a bioactive agent, a growth factor, a differentiation factor, a cytokine, or another polypeptide, gene product, or metabolic product of interest. Methods of genetically modifying cells for expressing and secreting compounds of interest are known in the art and readily utilized by those skilled in the art in the practice of the present invention.

In certain embodiments, differentiated cells that have been reprogrammed into stem cells can be used. To give but one example, human skin cells reprogrammed into embryonic stem cells by the transduction of Oct3/4, Sox2, c-Myc and Klf4 (see, for example, Junying, et al., Science, 318: 1917, 2007 and Takahashi et. al., Cell, 2007, 131:1, 2007).

In some embodiments, an additive for use in the practice of the present invention is or comprises a therapeutic agent. As used herein, the term “therapeutic agent” typically refers to a molecule, group of molecules, complex or substance that, when administered to an organism (e.g., according to a therapeutic regimen), achieves, or is expected to achieve (e.g., based on pre-clinical or clinical studies establishing an appropriate correlation) a particular diagnostic, therapeutic, and/or prophylactic result.

In some embodiments, a “therapeutic agent” may be or comprise a “drug” and/or a “vaccine.” In some embodiments, a therapeutic agent may be or comprise a human or animal pharmaceutical, treatment, remedy, nutraceutical, cosmeceutical, biological, diagnostic agent and/or contraceptive, including compositions useful in clinical and/or veterinary screening, prevention, prophylaxis, healing, wellness, detection, imaging, diagnosis, therapy, surgery, monitoring, cosmetics, prosthetics, forensics and the like. In some embodiments, a therapeutic agent may be or comprise an agricultural, workplace, military, industrial, and/or environmental therapeutic or remedy.

In some embodiments, a therapeutic agent may be or comprise, for example, an agent or entity that recognizes cellular receptors, membrane receptors, hormone receptors, therapeutic receptors, microbes, viruses or other selected targets in or on plant, animal and/or human cells.

In some embodiments, a therapeutic agent may provide a local and/or a systemic biological, physiological, or therapeutic effect in a biological system to which it is applied. For example, in some embodiments a therapeutic agent can act to control infection or inflammation, enhance cell growth and tissue regeneration, control tumor growth, act as an analgesic, promote anti-cell attachment, and enhance bone growth, among other functions. Alternatively or additionally, in some embodiments, a therapeutic agent may be or comprise an anti-viral agent, hormone, antibody, or therapeutic protein. In some embodiments, a therapeutic agent is or comprises a prodrug (e.g., an agent that is not biologically active when administered but, upon administration to a subject is converted to a biologically active agent through metabolism or some other mechanism).

In some embodiments, a therapeutic agent is or comprises an anti-viral agent, anesthetic, anticoagulant, anti-cancer agent, inhibitor of an enzyme, steroidal agent, anti-inflammatory agent, anti-neoplastic agent, antigen, vaccine, antibody, decongestant, antihypertensive, sedative, birth control agent, progestational agent, anti-cholinergic, analgesic, anti-depressant, anti-psychotic, β-adrenergic blocking agent, diuretic, cardiovascular active agent, vasoactive agent, anti-glaucoma agent, neuroprotectant, angiogenesis inhibitor, antibiotics, NSAIDs, glaucoma medications, angiogenesis inhibitors, and/or neuroprotective agents, etc.

In some embodiments, a therapeutic agent is or comprises an antibiotic, anti-viral agent, anesthetic, anticoagulant, anti-cancer agent, inhibitor of an enzyme, steroidal agent, anti-inflammatory agent, anti-neoplastic agent, antigen, vaccine, antibody, decongestant, antihypertensive, sedative, birth control agent, progestational agent, anti-cholinergic, analgesic, anti-depressant, anti-psychotic, β-adrenergic blocking agent, diuretic, cardiovascular active agent, vasoactive agent, anti-glaucoma agent, neuroprotectant, angiogenesis inhibitor, etc.

In various embodiments, a therapeutic agent be or include an compound or material of any chemical class including, for example, small organic or inorganic molecules; saccharines; oligosaccharides; polysaccharides; biological macromolecules, e.g., peptides, proteins, and peptide analogs and derivatives; peptidomimetics; antibodies and antigen binding fragments thereof; nucleic acids; nucleic acid analogs and derivatives; an extract made from biological materials such as bacteria, plants, fungi, or animal cells; animal tissues; naturally occurring or synthetic compositions; and any combinations thereof

In some embodiments, the therapeutic agent may be or comprise a small molecule.

In some embodiments, a therapeutic agent may be or comprise a polypeptide agent, e.g., an antibody agent.

In some embodiments, a therapeutic agent may be or comprise a nucleic acid agent such as, for example deoxyribonucleic acid (DNA), ribonucleic acid (RNA), nucleic acid analogues (e.g., locked nucleic acid (LNA), peptide nucleic acid (PNA), xeno nucleic acid (XNA)), or mixtures or combinations thereof, including, for example, DNA nanoplexes, siRNA, microRNA, shRNA, aptamers, ribozymes, decoy nucleic acids, antisense nucleic acids, RNA activators, and the like.

In some embodiments, a therapeutic agent is a drug indicated for the treatment a bone or tissue disease, for example, alendronate is indicated for the treatment of osteoporosis.

In some embodiments, a therapeutic agent is or comprises a mineral or mineral composite indicated for the treatment or reconstruction of bone or tissue, for example, hydroxyapatite as a supplement to induce bone growth or as a coating to promote bone ingrowth into prosthetic implants.

In some embodiments, a therapeutic agent may be a mixture of pharmaceutically active agents. For example, a local anesthetic may be delivered in combination with an anti-inflammatory agent such as a steroid. Local anesthetics may also be administered with vasoactive agents such as epinephrine. To give but another example, an antibiotic may be combined with an inhibitor of the enzyme commonly produced by bacteria to inactivate the antibiotic (e.g., penicillin and clavulanic acid).

Exemplary therapeutic agents include, but are not limited to, those found in Harrison's Principles of Internal Medicine, 13th Edition, Eds. T. R. Harrison et al. McGraw-Hill N.Y., NY; Physicians' Desk Reference, 50th Edition, 1997, Oradell New Jersey, Medical Economics Co.; Pharmacological Basis of Therapeutics, 8th Edition, Goodman and Gilman, 1990; United States Pharmacopeia, The National Formulary, USP XII NF XVII, 1990, the complete contents of all of which are incorporated herein by reference.

Therapeutic agents include the herein disclosed categories and specific examples. It is not intended that the category be limited by the specific examples. Those of ordinary skill in the art will recognize also numerous other compounds that fall within the categories and that are useful according to the present disclosure. Examples include a radiosensitizer, a steroid, a xanthine, a beta-2-agonist bronchodilator, an anti-inflammatory agent, an analgesic agent, a calcium antagonist, an angiotensin-converting enzyme inhibitors, a beta-blocker, a centrally active alpha-agonist, an alpha-1-antagonist, an anticholinergic/antispasmodic agent, a vasopressin analogue, an antiarrhythmic agent, an antiparkinsonian agent, an antiangina/antihypertensive agent, an anticoagulant agent, an antiplatelet agent, a sedative, an ansiolytic agent, a peptidic agent, a biopolymeric agent, an antineoplastic agent, a laxative, an antidiarrheal agent, an antimicrobial agent, an antifungal agent, a vaccine, a protein, or a nucleic acid. In a further aspect, the pharmaceutically active agent can be coumarin, albumin, steroids such as betamethasone, dexamethasone, methylprednisolone, prednisolone, prednisone, triamcinolone, budesonide, hydrocortisone, and pharmaceutically acceptable hydrocortisone derivatives; xanthines such as theophylline and doxophylline; beta-2-agonist bronchodilators such as salbutamol, fenterol, clenbuterol, bambuterol, salmeterol, fenoterol; antiinflammatory agents, including antiasthmatic anti-inflammatory agents, antiarthritis antiinflammatory agents, and non-steroidal antiinflammatory agents, examples of which include but are not limited to sulfides, mesalamine, budesonide, salazopyrin, diclofenac, pharmaceutically acceptable diclofenac salts, nimesulide, naproxene, acetaminophen, ibuprofen, ketoprofen and piroxicam; analgesic agents such as salicylates; calcium channel blockers such as nifedipine, amlodipine, and nicardipine; angiotensin-converting enzyme inhibitors such as captopril, benazepril hydrochloride, fosinopril sodium, trandolapril, ramipril, lisinopril, enalapril, quinapril hydrochloride, and moexipril hydrochloride; beta-blockers (i.e., beta adrenergic blocking agents) such as sotalol hydrochloride, timolol maleate, esmolol hydrochloride, carteolol, propanolol hydrochloride, betaxolol hydrochloride, penbutolol sulfate, metoprolol tartrate, metoprolol succinate, acebutolol hydrochloride, atenolol, pindolol, and bisoprolol fumarate; centrally active alpha-2-agonists such as clonidine; alpha-1-antagonists such as doxazosin and prazosin; anticholinergic/antispasmodic agents such as dicyclomine hydrochloride, scopolamine hydrobromide, glycopyrrolate, clidinium bromide, flavoxate, and oxybutynin; vasopressin analogues such as vasopressin and desmopressin; antiarrhythmic agents such as quinidine, lidocaine, tocainide hydrochloride, mexiletine hydrochloride, digoxin, verapamil hydrochloride, propafenone hydrochloride, flecainide acetate, procainamide hydrochloride, moricizine hydrochloride, and disopyramide phosphate; antiparkinsonian agents, such as dopamine, L-Dopa/Carbidopa, selegiline, dihydroergocryptine, pergolide, lisuride, apomorphine, and bromocryptine; antiangina agents and antihypertensive agents such as isosorbide mononitrate, isosorbide dinitrate, propranolol, atenolol and verapamil; anticoagulant and antiplatelet agents such as Coumadin, warfarin, acetylsalicylic acid, and ticlopidine; sedatives such as benzodiazapines and barbiturates; ansiolytic agents such as lorazepam, bromazepam, and diazepam; peptidic and biopolymeric agents such as calcitonin, leuprolide and other LHRH agonists, hirudin, cyclosporin, insulin, somatostatin, protirelin, interferon, desmopressin, somatotropin, thymopentin, pidotimod, erythropoietin, interleukins, melatonin, granulocyte/macrophage-CSF, and heparin; antineoplastic agents such as etoposide, etoposide phosphate, cyclophosphamide, methotrexate, 5-fluorouracil, vincristine, doxorubicin, cisplatin, hydroxyurea, leucovorin calcium, tamoxifen, flutamide, asparaginase, altretamine, mitotane, and procarbazine hydrochloride; laxatives such as senna concentrate, casanthranol, bisacodyl, and sodium picosulphate; antidiarrheal agents such as difenoxine hydrochloride, loperamide hydrochloride, furazolidone, diphenoxylate hdyrochloride, and microorganisms; vaccines such as bacterial and viral vaccines; antimicrobial agents such as penicillins, cephalosporins, and macrolides, antifungal agents such as imidazolic and triazolic derivatives; and nucleic acids such as DNA sequences encoding for biological proteins, and antisense oligonucleotides.

Anti-cancer agents include alkylating agents, platinum agents, antimetabolites, topoisomerase inhibitors, antitumor antibiotics, antimitotic agents, aromatase inhibitors, thymidylate synthase inhibitors, DNA antagonists, farnesyltransferase inhibitors, pump inhibitors, histone acetyltransferase inhibitors, metalloproteinase inhibitors, ribonucleoside reductase inhibitors, TNF alpha agonists/antagonists, endothelinA receptor antagonists, retinoic acid receptor agonists, immuno-modulators, hormonal and antihormonal agents, photodynamic agents, and tyrosine kinase inhibitors.

Antibiotics include aminoglycosides (e.g., gentamicin, tobramycin, netilmicin, streptomycin, amikacin, neomycin), bacitracin, corbapenems (e.g., imipenem/cislastatin), cephalosporins, colistin, methenamine, monobactams (e.g., aztreonam), penicillins (e.g., penicillin G, penicillinV, methicillin, natcillin, oxacillin, cloxacillin, dicloxacillin, ampicillin, amoxicillin, carbenicillin, ticarcillin, piperacillin, mezlocillin, azlocillin), polymyxin B, quinolones, and vancomycin; and bacteriostatic agents such as chloramphenicol, clindanyan, macrolides (e.g., erythromycin, azithromycin, clarithromycin), lincomyan, nitrofurantoin, sulfonamides, tetracyclines (e.g., tetracycline, doxycycline, minocycline, demeclocyline), and trimethoprim. Also included are metronidazole, fluoroquinolones, and ritampin.

Anti-depressants are substances capable of preventing or relieving depression. Examples of anti-depressants include imipramine, amitriptyline, nortriptyline, protriptyline, desipramine, amoxapine, doxepin, maprotiline, tranylcypromine, phenelzine, and isocarboxazide.

Antihistamines include pyrilamine, chlorpheniramine, and tetrahydrazoline.

Anti-inflammatory agents include corticosteroids, nonsteroidal anti-inflammatory drugs (e.g., aspirin, phenylbutazone, indomethacin, sulindac, tolmetin, ibuprofen, piroxicam, and fenamates), acetaminophen, phenacetin, gold salts, chloroquine, D-Penicillamine, methotrexate colchicine, allopurinol, probenecid, and sulfinpyrazone.

Anti-spasmodics include atropine, scopolamine, oxyphenonium, and papaverine.

Analgesics include aspirin, phenybutazone, idomethacin, sulindac, tolmetic, ibuprofen, piroxicam, fenamates, acetaminophen, phenacetin, morphine sulfate, codeine sulfate, meperidine, nalorphine, opioids (e.g., codeine sulfate, fentanyl citrate, hydrocodone bitartrate, loperamide, morphine sulfate, noscapine, norcodeine, normorphine, thebaine, nor-binaltorphimine, buprenorphine, chlomaltrexamine, funaltrexamione, nalbuphine, nalorphine, naloxone, naloxonazine, naltrexone, and naltrindole), procaine, lidocain, tetracaine and dibucaine.

Enzyme inhibitors are substances which inhibit an enzymatic reaction. Examples of enzyme inhibitors include edrophonium chloride, N-methylphysostigmine, neostigmine bromide, physostigmine sulfate, tacrine, tacrine, 1-hydroxy maleate, iodotubercidin, p-bromotetramiisole, 10-(alpha-diethylaminopropionyl)-phenothiazine hydrochloride, calmidazolium chloride, hemicholinium-3,3,5-dinitrocatechol, diacylglycerol kinase inhibitor I, diacylglycerol kinase inhibitor II, 3-phenylpropargylamine, N°-monomethyl-Larginine acetate, carbidopa, 3-hydroxybenzylhydrazine, hydralazine, clorgyline, deprenyl, hydroxylamine, iproniazid phosphate, 6-MeO-tetrahydro-9H-pyrido-indole, nialamide, pargyline, quinacrine, semicarbazide, tranylcypromine, N,N-diethylaminoethyl-2,2-diphenylvalerate hydrochloride, 3-isobutyl-1-methylxanthne, papaverine, indomethacind, 2-cyclooctyl-2-hydroxyethylamine hydrochloride, 2,3-dichloro-a-methylbenzylamine (DCMB), 8,9-dichloro-2,3,4,5-tetrahydro-1H-2-benzazepine hydrochloride, p-amino glutethimide, p-aminoglutethimide tartrate, 3-iodotyrosine, alpha-methyltyrosine, acetazolamide, dichlorphenamide, 6-hydroxy-2-benzothiazolesulfonamide, and allopurinol.

Hormones include estrogens (e.g., estradiol, estrone, estriol, diethylstibestrol, quinestrol, chlorotrianisene, ethinyl estradiol, mestranol), anti-estrogens (e.g., clomiphene, tamoxifen), progestins (e.g., medroxyprogesterone, norethindrone, hydroxyprogesterone, norgestrel), antiprogestin (mifepristone), androgens (e.g, testosterone cypionate, fluoxymesterone, danazol, testolactone), anti-androgens (e.g., cyproterone acetate, flutamide), thyroid hormones (e.g., triiodothyronne, thyroxine, propylthiouracil, methimazole, and iodixode), and pituitary hormones (e.g., corticotropin, sumutotropin, oxytocin, and vasopressin). Hormones are commonly employed in hormone replacement therapy and/or for purposes of birth control. Steroid hormones, such as prednisone, are also used as immunosuppressants and anti-inflammatories.

Muscle relaxants include mephenesin, methocarbomal, cyclobenzaprine hydrochloride, trihexylphenidyl hydrochloride, levodopa/carbidopa, and biperiden.

Ophthalmic agents include sodium fluorescein, rose bengal, methacholine, adrenaline, cocaine, atropine, alpha-chymotrypsin, hyaluronidase, betaxalol, pilocarpine, timolol, timolol salts, and combinations thereof

Prostaglandins are art recognized and are a class of naturally occurring chemically related long-chain hydroxy fatty acids that have a variety of biological effects.

Trophic factors are factors whose continued presence improves the viability or longevity of a cell. Trophic factors include, without limitation, platelet-derived growth factor (PDGP), neutrophil-activating protein, monocyte chemoattractant protein, macrophage-inflammatory protein, platelet factor, platelet basic protein, and melanoma growth stimulating activity; epidermal growth factor, transforming growth factor (alpha), fibroblast growth factor, platelet-derived endothelial cell growth factor, insulin-like growth factor, glial derived growth neurotrophic factor, ciliary neurotrophic factor, nerve growth factor, bone growth/cartilage-inducing factor (alpha and beta), bone morphogenetic proteins, interleukins (e.g., interleukin inhibitors or interleukin receptors, including interleukin 1 through interleukin 10), interferons (e.g., interferon alpha, beta and gamma), hematopoietic factors, including erythropoietin, granulocyte colony stimulating factor, macrophage colony stimulating factor and granulocyte-macrophage colony stimulating factor; tumor necrosis factors, and transforming growth factors (beta), including beta-1, beta-2, beta-3, inhibin, and activin.

As used herein, the term “small molecule” can refer to compounds that are “natural product-like,” however, the term “small molecule” is not limited to “natural product-like” compounds. Rather, a small molecule is typically characterized in that it contains several carbon-carbon bonds, and has a molecular weight of less than 5000 Daltons (5 kDa), preferably less than 3 kDa, still more preferably less than 2 kDa, and most preferably less than 1 kDa. In some cases it is preferred that a small molecule have a molecular weight equal to or less than 700 Daltons.

In some embodiments, a small molecule has a low molecular weight. In some embodiments, a low molecular weight being below about 100 Da, 200 Da, 300 Da, 400 Da, 0.5 kDa, 1 kDa, 1.5 kDa, 2 kDa, 3 kDa, 4 kDa, 5 kDa, 6 kDa, 7 kDa, 8 kDa, 9 kDa, or 10 kDa.

In some embodiments, a small molecule has pharmaceutical activity. In some embodiments, a small molecule is a clinically-used drug. In some embodiments, a small molecule is or comprises an antibiotic, anti-viral agent, anesthetic, anticoagulant, anti-cancer agent, inhibitor of an enzyme, steroidal agent, anti-inflammatory agent, anti-neoplastic agent, antigen, vaccine, antibody, decongestant, antihypertensive, sedative, birth control agent, progestational agent, anti-cholinergic, analgesic, anti-depressant, anti-psychotic, β-adrenergic blocking agent, diuretic, cardiovascular active agent, vasoactive agent, anti-glaucoma agent, neuroprotectant, angiogenesis inhibitor, etc.

In some embodiments, a small molecule may be an antibiotic. A non-exclusive list of antibiotics may include, but is not limited to, β-lactam antibiotics, macrolides, monobactams, rifamycins, tetracyclines, chloramphenicol, clindamycin, lincomycin, fusidic acid, novobiocin, fosfomycin, fusidate sodium, capreomycin, colistimethate, gramicidin, minocycline, doxycycline, bacitracin, erythromycin, nalidixic acid, vancomycin, and trimethoprim. For example, β-lactam antibiotics can be ampicillin, aziocillin, aztreonam, carbenicillin, cefoperazone, ceftriaxone, cephaloridine, cephalothin, cloxacillin, moxalactam, penicillin G, piperacillin, ticarcillin and any combination thereof

An antibiotic used in accordance with the present disclosure may be bacteriocidial or bacteriostatic. Other anti-microbial agents may also be used in accordance with the present disclosure. For example, anti-viral agents, anti-protazoal agents, anti-parasitic agents, etc. may be of use.

In some embodiments, a small molecule may be or comprise an anti-inflammatory agent. A non-exclusive list of anti-inflammatories may include, but is not limited to, corticosteroids (e.g., glucocorticoids), cycloplegics, non-steroidal anti-inflammatory drugs (NSAIDs), immune selective anti-inflammatory derivatives (ImSAIDs), and any combination thereof. Exemplary NSAIDs include, but not limited to, celecoxib (Celebrex®); rofecoxib (Vioxx®), etoricoxib (Arcoxia®), meloxicam (Mobic®), valdecoxib, diclofenac (Voltaren®, Cataflam®), etodolac (Lodine®), sulindac (Clinori®), aspirin, alclofenac, fenclofenac, diflunisal (Dolobid®), benorylate, fosfosal, salicylic acid including acetylsalicylic acid, sodium acetylsalicylic acid, calcium acetylsalicylic acid, and sodium salicylate; ibuprofen (Motrin), ketoprofen, carprofen, fenbufen, flurbiprofen, oxaprozin, suprofen, triaprofenic acid, fenoprofen, indoprofen, piroprofen, flufenamic, mefenamic, meclofenamic, niflumic, salsalate, rolmerin, fentiazac, tilomisole, oxyphenbutazone, phenylbutazone, apazone, feprazone, sudoxicam, isoxicam, tenoxicam, piroxicam (Feldene®), indomethacin (Indocin®), nabumetone (Relafen®), naproxen (Naprosyn®), tolmetin, lumiracoxib, parecoxib, licofelone (ML3000), including pharmaceutically acceptable salts, isomers, enantiomers, derivatives, prodrugs, crystal polymorphs, amorphous modifications, co-crystals and combinations thereof

Those skilled in the art will recognize that this is an exemplary, not comprehensive, list of small molecules that can be released using compositions and methods in accordance with the present disclosure. In addition to a therapeutic agent or alternatively, various other agents may be associated with a coated substrate in accordance with the present disclosure.

In some embodiments, the additive is an agent that stimulates tissue formation, and/or healing and regrowth of natural tissues, and any combinations thereof. Agents that increase formation of new tissues and/or stimulates healing or regrowth of native tissue at the site of injection can include, but are not limited to, fibroblast growth factor (FGF), transforming growth factor-beta (TGF-beta, platelet-derived growth factor (PDGF), epidermal growth factors (EGFs), connective tissue activated peptides (CTAPs), osteogenic factors including bone morphogenic proteins, heparin, angiotensin II (A-II) and fragments thereof, insulin-like growth factors, tumor necrosis factors, interleukins, colony stimulating factors, erythropoietin, nerve growth factors, interferons, biologically active analogs, fragments, and derivatives of such growth factors, and any combinations thereof

In some embodiments, a bio-ink composition, e.g., silk and glycerol, composition can further comprise at least one additional material for soft tissue augmentation, e.g., dermal filler materials, including, but not limited to, poly(methyl methacrylate) microspheres, hydroxylapatite, poly(L-lactic acid), collagen, elastin, and glycosaminoglycans, hyaluronic acid, commercial dermal filler products such as BOTOX® (from Allergan), DYSPORT®, COSMODERM®, EVOLENCE®, RADIESSE®, RESTYLANE®, JUVEDERM® (from Allergan), SCULPTRA®, PERLANE®, and CAPTIQUE®, and any combinations thereof

In some embodiments, the additive is a wound healing agent. As used herein, a “wound healing agent” is a compound or composition that actively promotes wound healing process. Exemplary wound healing agents include, but are not limited to dexpanthenol; growth factors; enzymes, hormones; povidon-iodide; fatty acids; anti-inflammatory agents; antibiotics; antimicrobials; antiseptics; cytokines; thrombin; angalgesics; opioids; aminoxyls; furoxans; nitrosothiols; nitrates and anthocyanins; nucleosides, such as adenosine; and nucleotides, such as adenosine diphosphate (ADP) and adenosine triphosphate (ATP); neutotransmitter/neuromodulators, such as acetylcholine and 5-hydroxytryptamine (serotonin/5-HT); histamine and catecholamines, such as adrenalin and noradrenalin; lipid molecules, such as sphingosine-1-phosphate and lysophosphatidic acid; amino acids, such as arginine and lysine; peptides such as the bradykinins, substance P and calcium gene-related peptide (CGRP); nitric oxide; and any combinations thereof

In certain embodiments, the active agents described herein are immunogens. In some embodiments, the immunogen is a vaccine. Most vaccines are sensitive to environmental conditions under which they are stored and/or transported. For example, freezing may increase reactogenicity (e.g., capability of causing an immunological reaction) and/or loss of potency for some vaccines (e.g., HepB, and DTaP/IPV/HIB), or cause hairline cracks in the container, leading to contamination. Further, some vaccines (e.g., BCG, Varicella, and MMR) are sensitive to heat. Many vaccines (e.g., BCG, MMR, Varicella, Meningococcal C Conjugate, and most DTaP-containing vaccines) are light sensitive. See, e.g., Galazka et al., Thermostability of vaccines, in Global Programme for Vaccines & Immunization (World Health Organization, Geneva, 1998); Peetermans et al., Stability of freeze-dried rubella virus vaccine (Cendehill strain) at various temperatures, 1 J. Biological Standardization 179 (1973). Thus, the compositions and methods described herein also provide for stabilization of vaccines regardless of the cold chain and/or other environmental conditions.

In some embodiments, a therapeutic agent is or comprises a growth factor. In some embodiments, a useful growth factor is or comprises BMP, PDGF, VEGF, and/or PDGF. In some embodiments, a growth factor is or includes, for example, adrenomedullin, angiopoietin, autocrine motility factor, basophils, brain-derived neurotrophic factor, bone morphogenetic protein, colony-stimulating factors, connective tissue growth factor, endothelial cells, epidermal growth factor, erythropoietin, fibroblast growth factor, fibroblasts, glial cell line-derived neurotrophic factor, granulocyte colony stimulating factor, granulocyte macrophage colony stimulating factor, growth differentiation factor-9, hepatocyte growth factor, hepatoma-derived growth factor, insulin-like growth factor, interleukins, keratinocyte growth factor, keratinocytes, lymphocytes, macrophages, mast cells, myostatin, nerve growth factor, neurotrophins, platelet-derived growth factor, placenta growth factor, osteoblasts, platelets, proinflammatory, stromal cells, T-lymphocytes, thrombopoietin, transforming growth factor alpha, transforming growth factor beta, tumor necrosis factor-alpha, vascular endothelial growth factor and combinations thereof

Some embodiments of the present invention can be particularly useful for healing bone and/or tissue defects or reconstructing bone and/or tissue. Exemplary agents useful as growth factor for defect repair and/or healing can include, but are not limited to, growth factors for defect treatment modalities now known in the art or later-developed; exemplary factors, agents or modalities including natural or synthetic growth factors, cytokines, or modulators thereof to promote bone and/or tissue defect healing. Suitable examples may include, but not limited to 1) topical or dressing and related therapies and debriding agents (such as, for example, Santyl® collagenase) and Iodosorb® (cadexomer iodine); 2) antimicrobial agents, including systemic or topical creams or gels, including, for example, silver-containing agents such as SAGs (silver antimicrobial gels), (CollaGUARD™, Innocoll, Inc) (purified type-I collagen protein based dressing), CollaGUARD Ag (a collagen-based bioactive dressing impregnated with silver for infected wounds or wounds at risk of infection), DermaSIL™ (a collagen-synthetic foam composite dressing for deep and heavily exuding wounds); 3) cell therapy or bioengineered skin, skin substitutes, and skin equivalents, including, for example, Dermograft (3-dimensional matrix cultivation of human fibroblasts that secrete cytokines and growth factors), Apligraf® (human keratinocytes and fibroblasts), Graftskin® (bilayer of epidermal cells and fibroblasts that is histologically similar to normal skin and produces growth factors similar to those produced by normal skin), TransCyte (a Human Fibroblast Derived Temporary Skin Substitute) and Oasis® (an active biomaterial that comprises both growth factors and extracellular matrix components such as collagen, proteoglycans, and glycosaminoglycans); 4) cytokines, growth factors or hormones (both natural and synthetic) introduced to the wound to promote wound healing, including, for example, NGF, NT3, BDGF, integrins, plasmin, semaphoring, blood-derived growth factor, keratinocyte growth factor, tissue growth factor, TGF-alpha, TGF-beta, PDGF (one or more of the three subtypes may be used: AA, AB, and B), PDGF-BB, TGF-beta 3, factors that modulate the relative levels of TGFβ3, TGFβ1, and TGFβ2 (e.g., Mannose-6-phosphate), sex steroids, including for example, estrogen, estradiol, or an oestrogen receptor agonist selected from the group consisting of ethinyloestradiol, dienoestrol, mestranol, oestradiol, oestriol, a conjugated oestrogen, piperazine oestrone sulphate, stilboestrol, fosfesterol tetrasodium, polyestradiol phosphate, tibolone, a phytoestrogen, 17-beta-estradiol; thymic hormones such as Thymosin-beta-4, EGF, HB-EGF, fibroblast growth factors (e.g., FGF1, FGF2, FGF7), keratinocyte growth factor, TNF, interleukins family of inflammatory response modulators such as, for example, IL-10, IL-1, IL-2, IL-6, IL-8, and IL-10 and modulators thereof; INFs (INF-alpha, -beta, and -delta); stimulators of activin or inhibin, and inhibitors of interferon gamma prostaglandin E2 (PGE2) and of mediators of the adenosine 3′,5′-cyclic monophosphate (cAMP) pathway; adenosine A1 agonist, adenosine A2 agonist or 5) other agents useful for wound healing, including, for example, both natural or synthetic homologues, agonist and antagonist of VEGF, VEGFA, IGF; IGF-1, proinflammatory cytokines, GM-CSF, and leptins and 6) IGF-1 and KGF cDNA, autologous platelet gel, hypochlorous acid (Sterilox® lipoic acid, nitric oxide synthase3, matrix metalloproteinase 9 (MMP-9), CCT-ETA, alphavbeta6 integrin, growth factor-primed fibroblasts and Decorin, silver containing wound dressings, Xenaderm™, papain wound debriding agents, lactoferrin, substance P, collagen, and silver-ORC, placental alkaline phosphatase or placental growth factor, modulators of hedgehog signaling, modulators of cholesterol synthesis pathway, and APC (Activated Protein C), keratinocyte growth factor, TNF, Thromboxane A2, NGF, BMP bone morphogenetic protein, CTGF (connective tissue growth factor), wound healing chemokines, decorin, modulators of lactate induced neovascularization, cod liver oil, placental alkaline phosphatase or placental growth factor, and thymosin beta 4. In certain embodiments, one, two three, four, five or six agents useful for wound healing may be used in combination. More details can be found in U.S. Pat. No. 8,247,384, the contents of which are incorporated herein by reference.

It is to be understood that agents useful for growth factor for healing (including for example, growth factors and cytokines) above encompass all naturally occurring polymorphs (for example, polymorphs of the growth factors or cytokines). Also, functional fragments, chimeric proteins comprising one of said agents useful for wound healing or a functional fragment thereof, homologues obtained by analogous substitution of one or more amino acids of the wound healing agent, and species homologues are encompassed. It is contemplated that one or more agents useful for wound healing may be a product of recombinant DNA technology, and one or more agents useful for wound healing may be a product of transgenic technology. For example, platelet derived growth factor may be provided in the form of a recombinant PDGF or a gene therapy vector comprising a coding sequence for PDGF.

Those skilled in the art will recognize that this is an exemplary, not comprehensive, list of additives, dopants, and agents that can be utilized in accordance with the present invention.

Pigments and/or Dyes

In some embodiments, bio-ink composition compositions utilized in accordance with the present invention can include a colorant, such as a pigment or dye or combination thereof

In general, any organic and/or inorganic pigments and dyes can be included in the inks. Exemplary pigments suitable for use in the present invention include International Color Index or C.I. Pigment Black Numbers 1, 7, 11 and 31, C.I. Pigment Blue Numbers 15, 15:1, 15:2, 15:3, 15:4, 15:6, 16, 27, 29, 61 and 62, C.I. Pigment Green Numbers 7, 17, 18 and 36, C.I. Pigment Orange Numbers 5, 13, 16, 34 and 36, C.I. Pigment Violet Numbers 3, 19, 23 and 27, C.I. Pigment Red Numbers 3, 17, 22, 23, 48:1, 48:2, 57:1, 81:1, 81:2, 81:3, 81:5, 101, 1 14, 122, 144, 146, 170, 176, 179, 181, 185, 188, 202, 206, 207, 210 and 249, C.I. Pigment Yellow Numbers 1, 2, 3, 12, 13, 14, 17, 42, 65, 73, 74, 75, 83, 93, 109, 1 10, 128, 138, 139, 147, 142, 151, 154 and 180, D&C Red No. 7, D&C Red No. 6 and D&C Red No. 34, carbon black pigment (such as Regal 330, Cabot Corporation), quinacridone pigments (Quinacridone Magenta (228-0122), available from Sun Chemical Corporation, Fort Lee, N.J.), diarylide yellow pigment (such as AAOT Yellow (274-1788) available from Sun Chemical Corporation); and phthalocyanine blue pigment (such as Blue 15:3 (294-1298) available from Sun Chemical Corporation).

Classes of dyes suitable for use in present invention can be selected from acid dyes, natural dyes, direct dyes (either cationic or anionic), basic dyes, and reactive dyes. The acid dyes, also regarded as anionic dyes, are soluble in water and mainly insoluble in organic solvents and are selected, from yellow acid dyes, orange acid dyes, red acid dyes, violet acid dyes, blue acid dyes, green acid dyes, and black acid dyes.

European Patent 0745651, incorporated herein by reference, describes a number of acid dyes that are suitable for use in the present invention. Exemplary yellow acid dyes include Acid Yellow 1 International Color Index or C.I. 10316); Acid Yellow 7 (C.I. 56295); Acid Yellow 17 (C.I. 18965); Acid Yellow 23 (C.I. 19140); Acid Yellow 29 (C.I. 18900); Acid Yellow 36 (C.I. 13065); Acid Yellow 42 (C.I. 22910); Acid Yellow 73 (C.I. 45350); Acid Yellow 99 (C.I. 13908); Acid Yellow 194; and Food Yellow 3 (C.I. 15985). Exemplary orange acid dyes include Acid Orange 1 (C.I. 13090/1); Acid Orange 10 (C.I. 16230); Acid Orange 20 (C.I. 14603); Acid Orange 76 (C.I. 18870); Acid Orange 142; Food Orange 2 (C.I. 15980); and Orange B.

Exemplary red acid dyes include Acid Red 1. (C.I. 18050); Acid Red 4 (C.I. 14710); Acid Red 18 (C.I. 16255), Acid Red 26 (C.I. 16150); Acid Red 2.7 (C.I. as Acid Red 51 (C.I. 45430, available from BASF Corporation, Mt. Olive, N.J.) Acid Red 52 (C.I. 45100); Acid Red 73 (C.I. 27290); Acid Red 87 (C. I. 45380); Acid Red 94 (C.I. 45440) Acid Red 194; and Food Red 1 (C.I. 14700). Exemplary violet acid dyes include Acid Violet 7 (C.I. 18055); and Acid Violet 49 (C.I. 42640). Exemplary blue acid dyes include Acid Blue 1 (C.I. 42045); Acid Blue 9 (C.I. 42090); Acid Blue 22 (C.I. 42755); Acid Blue 74 (C.I. 73015); Acid Blue 93 (C.I. 42780); and Acid Blue 158A (C.I. 15050). Exemplary green acid dyes include Acid Green 1 (C.I. 10028); Acid Green 3 (C.I. 42085); Acid Green 5 (C.I. 42095); Acid Green 26 (C.I. 44025); and Food Green 3 (C.I. 42053). Exemplary black acid dyes include Acid Black 1 (C.I. 20470); Acid Black 194 (Basantol® X80, available from BASF Corporation, an azo/1:2 CR-complex.

Exemplary direct dyes for use in the present invention include Direct Blue 86 (C.I. 74180); Direct Blue 199; Direct Black 168; Direct Red 253; and Direct Yellow 107/132 (C.I. Not Assigned).

Exemplary natural dyes for use in the present invention include Alkanet (C.I. 75520,75530); Annafto (C.I. 75120); Carotene (C.I. 75130); Chestnut; Cochineal (C.I.75470); Cutch (C.I. 75250, 75260); Divi-Divi; Fustic (C.I. 75240); Hypernic (C.I. 75280); Logwood (C.I. 75200); Osage Orange (C.I. 75660); Paprika; Quercitron (C.I. 75720); Sanrou (C.I. 75100); Sandal Wood (C.I. 75510, 75540, 75550, 75560); Sumac; and Tumeric (C.I. 75300). Exemplary reactive dyes for use in the present invention include Reactive Yellow 37 (monoazo dye); Reactive Black 31 (disazo dye); Reactive Blue 77 (phthalo cyanine dye) and Reactive Red 180 and Reactive Red 108 dyes. Suitable also are the colorants described in The Printing Ink Manual (5th ed., Leach et al. eds. (2007), pages 289-299. Other organic and inorganic pigments and dyes and combinations thereof can be used to achieve the colors desired.

In addition to or in place of visible colorants, UV fluorophores that are excited in the UV range and emit light at a higher wavelength (typically 400 nm and above) can be utilized in accordance with the present invention. Examples of UV fluorophores include but are not limited to materials from the coumarin, benzoxazole, rhodamine, napthalimide, perylene, benzanthrones, benzoxanthones or benzothia-xanthones families. In some embodiments, addition of a UV fluorophore (such as an optical brightener for instance) can help maintain maximum visible light transmission.

In many embodiments, the amount of colorant, when present, generally is between 0.05% and 5% or between 0.1% and 1% based on the weight of the bio-ink composition.

For non-white inks, the amount of pigment/dye generally is present in an amount of from at or about 0.1 wt % to at or about 20 wt % based on the weight of the bio-ink composition. In some applications, a non-white ink can include 15 wt % or less pigment/dye, or 10 wt % or less pigment/dye or 5 wt % pigment/dye, or 1 wt % pigment/dye based on the weight of the ink composition. In some applications, a non-white ink can include 1 wt % to 10 wt %, or 5 wt % to 15 wt %, or 10 wt % to 20 wt % pigment/dye based on the weight of the bio-ink composition. In some applications, a non-white ink can contain an amount of dye/pigment that is 1 wt %, 2 wt %, 3 wt %, 4 wt %, 5%, 6 wt %, 7 wt %, 8 wt %, 9 wt %, 10 wt %, 11 wt %, 12 wt %, 13 wt %, 14 wt %, 15%, 16 wt %, 17 wt %, 18 wt %, 19 wt % or 20 wt % based on the weight of the bio-ink composition.

In many embodiments of white ink compositions, the amount of white pigment generally is present in an amount of from at or about 1 wt % to at or about 60 wt % based on the weight of the bio-ink composition. In some applications, greater than 60 wt % white pigment can be present. Preferred white pigments include titanium dioxide (anatase and rutile), zinc oxide, lithopone (calcined coprecipitate of barium sulfate and zinc sulfide), zinc sulfide, blanc fixe and alumina hydrate and combinations thereof, although any of these can be combined with calcium carbonate. In some applications, a white ink can include 60 wt % or less white pigment, or 55 wt % or less white pigment, or 50 wt % white pigment, or 45 wt % white pigment, or 40 wt % white pigment, or 35 wt % white pigment, or 30 wt % white pigment, or 25 wt % white pigment, or 20 wt % white pigment, or 15 wt % white pigment, or 10 wt % white pigment, based on the weight of the ink composition. In some applications, a white ink can include 5 wt % to 60 wt %, or 5 wt % to 55 wt %, or 10 wt % to 50 wt %, or 10 wt % to 25 wt %, or 25 wt % to 50 wt %, or 5 wt % to 15 wt %, or 40 wt % to 60 wt % white pigment based on the weight of the ink composition. In some applications, a non-white ink can an amount of dye/pigment that is 5%, 6 wt %, 7 wt %, 8 wt %, 9 wt %, 10 wt %, 11 wt %, 12 wt %, 13 wt %, 14 wt %, 15%, 16 wt %, 17 wt %, 18 wt %, 19 wt %, 20 wt %, 21 wt %, 22 wt %, 23 wt %, 24 wt %, 25%, 26 wt %, 27 wt %, 28 wt %, 29 wt %, 30 wt %, 31 wt %, 32 wt %, 33 wt %, 34 wt %, 35%, 36 wt %, 37 wt %, 38 wt %, 39 wt %, 40 wt %, 41 wt %, 42 wt %, 43 wt %, 44 wt %, 45%, 46 wt %, 47 wt %, 48 wt %, 49 wt %, 50 wt %, 51 wt %, 52 wt %, 53 wt %, 54 wt %, 55%, 56 wt %, 57 wt %, 58 wt %, 59 wt % or 60 wt % based on the weight of the ink composition.

Methods of Preparing Bio-ink compositions

In some embodiments, bio-ink compositions for use in accordance with the present invention are manufactured from methods according to the present invention. In some embodiments, methods of providing, preparing, and/or manufacturing bio-ink compositions for use in accordance with the present invention include a polypeptide (e.g. silk, such as silk fibroin) solution. In some embodiments, a polypeptide solution comprises a solution of actins, a solution of catenins, a solution of claudins, a solution of coilins, a solution of collagen, a solution of elastin, a solution of elaunins, a solution of extensins, a solution of fibroins, a solution of fibrillins, a solution of keratins, a solution of lamins, a solution of laminins, a solution of silks, a solution of tublins, a solution of viral structural proteins, a solution of zein proteins (seed storage protein) and any combinations thereof

While silk fibroin extraction methods generally have been well documented, example embodiments of the present invention encompass the recognition that certain polypeptides can be processed further to be made suitable for 3D bio-printing as described herein.

In some embodiments, a bio-ink composition for use in the practice of the present invention is provided, prepared, and/or manufactured by boiling a polypeptide, such as a silk, in a solution for example of in Na₂CO₃. In some embodiments, boiling is performed at a temperature within the range of: about 30° C., about 35° C., about 40° C., about 45° C., about 50° C., about 45° C., about 60° C., about 65° C., about 70° C., about 75° C., about 80° C., about 85° C., about 90° C., about 95° C., about 100° C., about 105° C., about 110° C., about 115° C., about at least 120° C. In some embodiments, methods involve extraction of polypeptides (such as silk fibroin) under high temperature, such as between about 101 and about 135° C., between about 105 and about 130° C., between about 110 and about 130° C., between about 115 and about 125° C., between about 118 and about 123° C., e.g., about 115° C., 116° C., 117° C., 118° C., 119° C., 120° C., 121° C., 122° C., 123° C., 124° C., 125° C. In some embodiments, boiling is performed at a temperature below about 65° C. In some embodiments, boiling is performed at a temperature of about 60° C. or less.

In some embodiments, degumming is performed at a temperature of: about 30° C., about 35° C., about 40° C., about 45° C., about 50° C., about 55° C., about 60° C., about 65° C., about 70° C., about 75° C., about 80° C., about 85° C., about 90° C., about 95° C., about 100° C., about 105° C., about 110° C., about 115° C., about 120° C., about 125° C., about 130° C., about 135° C., about 140° C., about 145° C., or about at least 150° C.

Additionally or alternatively, provided methods in some embodiments, involve extraction of polypeptides (such as silk fibroin) under elevated pressure, such as about 5 psi, 6 psi, 7 psi, 8 psi, 9 psi, 10 psi, 11 psi, 12 psi, 13 psi, 14 psi, 15 psi, 16 psi, 17 psi, 18 psi, 19 psi, 20 psi, 21 psi, 22 psi, 23 psi, 24 psi, 25 psi, 30 psi, 31 psi, 32 psi, 33 psi, 34 psi and 35 psi. In some embodiments, polypeptides (such as silk fibroin) are extracted under high temperature and under elevated pressure, e.g., at about 110 and about 130° C. and about 10 and about 20 psi for a duration suitable to produce a polypeptide solution that would easily go through a 0.2 μm filter. In some embodiments, polypeptides (such as silk fibroin) are extracted under high temperature and under elevated pressure, e.g., at about 110° C. and about 130° C. and about 10 to about 20 psi for about 60 to about 180 minutes. In some embodiments, polypeptides (such as silk fibroin) are extracted under high temperature and under elevated pressure, e.g., at about 116° C. to about 126° C. and about 12 psi and about 20 psi for about 90 to about 150 minutes.

In some embodiments, dissolving silk in a solution is performed at a temperature of: about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., about 95° C., about at least 100° C.

In some embodiments, dialysis of a silk solution is performed at a temperature of: about 5° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 35° C., about 40° C., about 45° C., or about at least 50° C.

In some embodiments, polypeptides and/or polypeptide fragments for use in the practice of the present invention are produced having a molecular weight inversely related to a length of boiling time. In some particular embodiments, a bio-ink composition for use in accordance with the present invention is provided, prepared, and/or manufactured from a solution of silk fibroin that has been boiled for at least about 5, 10, 15, 20, 25, 30, 35, 40, 45, 50, 55, 60, 70, 80, 90, 100, 110, 120, 150, 180, 210, 240, 270, 310, 340, 370, 410 minutes or more.

In some embodiments, a bio-ink composition for use in accordance with the present invention is provided, prepared, and/or manufactured from a solution of a polypeptide having a molecular weight in the range of about 20 kD-about 400 kD. In some embodiments, provided, prepared, and/or manufactured bio-ink compositions for use in accordance with the present invention are comprised of polypeptides having molecular weights within a range between a lower bound (e.g., about 20 kD, about 30 kD, about 40 kD, about 50 kD, about 60 kD, or more) and an upper bound (e.g., about 400 kD, about 375 kD, about 350 kD, about 325 kD, about 300 kD, or less). In some embodiments, provided, prepared, and/or manufactured bio-ink compositions are comprised of polypeptide having a molecular weight around 60 kD.

In some embodiments, bio-ink compositions are provided, prepared, and/or manufactured from a polypeptide solution, such as a silk fibroin solution of about 0.5 wt % polypeptide to about 30 wt % polypeptide. In some embodiments, a bio-ink composition for use in accordance with the present invention is provided, prepared, and/or manufactured from a polypeptide solution, such as a silk fibroin solution that is less than about 30 wt % polypeptide. In some embodiments, bio-ink compositions for use in accordance with the present invention are provided, prepared, and/or manufactured from a polypeptide solution, such as a silk fibroin solution that is less than about 20 wt % polypeptide. In some embodiments, a bio-ink composition for use in accordance with the present invention is provided, prepared, and/or manufactured from a polypeptide solution, such as a silk fibroin solution that is less than about 10 wt % polypeptide. In some embodiments, bio-ink compositions for use in accordance with the present invention are provided, prepared, and/or manufactured from a polypeptide solution, such as a silk fibroin solution that is less than about 10 wt % polypeptide, or even that is about 5% wt %, about 4 wt %, about 3 wt %, about 2 wt %, about 1 wt % polypeptide or less.

In some embodiments, bio-ink compositions for use in the practice of the present invention are provided, prepared, and/or manufactured from an aqueous solution of polypeptide (e.g., silk polymer) where the solvent is water, PBS and combinations thereof. In some embodiments, a bio-ink composition for use in accordance with the present invention is provided, prepared, and/or manufactured from an aqueous polypeptide solution in a solvent other than PBS. In some embodiments, a bio-ink composition for use in accordance with the present invention is provided, prepared, and/or manufactured from a solution of polypeptide in water. In some embodiments, a bio-ink composition for use in accordance with the present invention is provided, prepared, and/or manufactured from a solution of polypeptide in DMEM. In some embodiments, a bio-ink composition for use in accordance with the present invention is provided, prepared, and/or manufactured from an aqueous polypeptide solution that is not buffered.

In some embodiments, provided bio-ink compositions are provided, prepared, and/or manufactured from silk fibers were solubilized in LiBr and then dialyzed against water. In some embodiments, bio-ink compositions for use in accordance with the present invention are provided, prepared, and/or manufactured from a silk solution adjusted and/or maintained at a sub-physiological pH. For example, in some embodiments, a bio-ink composition for use in accordance with the present invention is provided, prepared, and/or manufactured from a solution of polypeptide that is adjusted to and/or maintained at a pH near or below about 6. In some embodiments, bio-ink compositions are provided, prepared, and/or manufactured from a solution of protein polymer with a pH for instance about 6 or less, about 5 or less, about 4 or less, about 3 or less, about 2 or less, about 1.5 or less, or about 1 or less. However, in some alternative embodiments, bio-ink compositions are provided, prepared, and/or manufactured from a solution of protein polymer with a pH in a range for example of at least 6, at least 7, at least 8, at least 9, and at least about 10.

In some embodiments, aqueous silk fibroin solutions were prepared following procedures; obtaining about 40 mL of silk solution with a concentration of about 6.25% (wt/vol), if more volumes are needed, the materials can be scaled appropriately; cutting about 10 grams Bombyx mori silk cocoons into about half-dime-sized pieces while disposing of silkworms; measure about 8.5 gram of sodium carbonate; adding sodium carbonate into about 4 liters of water to prepare an about 0.02 M solution; placing a beaker containing an aqueous silk fibroin solution into an autoclave; setting an autoclave containing an aqueous silk fibroin solution to run at 121° C. under the pressure of 16 psi for 120 minutes; removing silk fibroin with a strainer; cooling silk fibroin by rinsing in ultrapure cold water for 20 minutes and repeating twice for a total of three rinses; removing silk fibroin and squeezing water from it; spreading squeezed silk fibroin out and allowing it to dry in a fume hood for about 12 hours, resulting in silk fibroin weighing slightly over 2.5 gram; dissolve 2.5 grams of silk fibroin into 10 mL of 9.3 M lithium bromide; stirring silk fibroin until completely dissolved; inserting about 10 mL of silk-LiBr solution into a pre-wet dialysis cassette between about 3 mL and about 12 mL; dialyzing against about 1 liter of ultrapure water for about 48 hours; removing silk from a pre-wet dialysis cassette; and place silk solution in a centrifuge and spin at 9,000 r.p.m. at 2° C. for 60 minutes, and storing centrifuged silk solution (about 40 mL of silk solution with a concentration of about 6.25%) in a refrigerator at about 4° C.

In some embodiments, aqueous silk fibroin solutions were prepared following published procedures, for example including those published in M. L. Lovett, et al., 28 Biomaterials 5271 (2007), which is hereby incorporated by reference in its entirety herein.

Another aspect of the invention provides methods for preparing bio-ink compositions, such as silk fibroin inks. An exemplary protocol for preparing a silk fibroin ink in accordance with the present disclosure is provided below.

In some embodiments, a polypeptide and a humectant are combine by blending and/or mixing. In some embodiments, bio-ink compositions are formed when combined in a polypeptide solution, such as a silk fibroin solution or when polypeptides are otherwise introduced into a silk matrix.

In some embodiments, glycerol is used in the material and is a simple metabolizable non-toxic sugar alcohol ubiquitous in food and pharmaceutical industries. When blended, glycerol stabilizes an intermediate conformation of crystallized silk which produces a more flexible yet stable and strong film. S. Lu et al., 11 Biomacromolecules, 143 (2010), which is hereby incorporated by reference in its entirety herein teaches methods for blending polypeptides and humectants and specifically blending silk with glycerol.

### Printed Articles

As described herein, the present invention provides articles. In some embodiments, articles are formed from bio-ink compositions as disclosed herein. In some embodiments, articles are formed by printing, depositing, and/or extruding a bio-ink composition. In some embodiments, articles are formed using printing and/or extruding technologies as described herein.

Structure

In some embodiments, an article forms when a bio-ink composition used in accordance with the present invention cures. In some embodiments as above described, bio-ink compositions in accordance with the present invention are printed, extruded, and/or deposited. In some embodiments, an article forms when a bio-ink composition such as those described herein is printed, deposited, and/or extruded on a printable surface. In some embodiments, an article forms when a bio-ink composition that was printed, deposited, and/or extruded cures.

In some embodiments, a printed article is homogenous. In some embodiments, a printed article comprises one or more printed layers formed from a same cured bio-ink composition. In some embodiments, a printed article is heterogeneous. In some embodiments, a printed article comprises more than one printed layer formed from different cured bio-ink compositions. In some embodiments, bio-ink compositions comprise different agents or additives. In some embodiments, bio-ink compositions comprise a different polypeptide. In some embodiments, bio-ink compositions comprise a polypeptide have a different molecular weight. In some embodiments, bio-ink compositions comprise a different humectant. In some embodiments, bio-ink compositions comprise a polypeptide having a different concentration. In some embodiments, bio-ink compositions comprise a different humectant having a different concentration. In some embodiments, bio-ink compositions comprise a different ratio of a polypeptide:humectant.

In some embodiments, printed articles formed from bio-ink compositions in accordance with the present invention comprising agents or additives that provide or contribute to one or more desirable properties (as described herein) of the bio-ink composition and/or of an article printed therewith, e.g., strength, flexibility, ease of processing and handling, biocompatibility, bioresorability, surface morphology, release rates and/or kinetics of one or more active agents present, and the like. In some embodiments in accordance with the present invention, multiple extruders are configured to deposit multiple bio-ink compositions.

In some embodiments, a bio-ink composition cures to form a solid or substantially solid article. In some embodiments, a solid or substantially solid article is crystalline. In some embodiments, an article is characterized by a beta-sheet secondary structure. In some embodiments, a bio-ink composition cures to form a partially solid article. In some embodiments, a partially solid article is crystalline. In some embodiments, a partially solid article is characterized by alpha helical and beta-sheet structure.

In some embodiments, bio-ink compositions for use in accordance with the present invention when printed, extruded, and/or deposited generate 2D structures that possess consistent geometry and regular features, including sharp angles and clean edges.

In some embodiments, 3D structures formed from bio-ink compositions for use in accordance with the present invention have consistent geometry and/or regular features, including sharp angles and clean edges. In some embodiments, 3D structures formed from bio-ink compositions for use in accordance with the present invention possess both geometry and features that can be maintained during exposure to subsequent printings, solvents, and/or physiological environments.

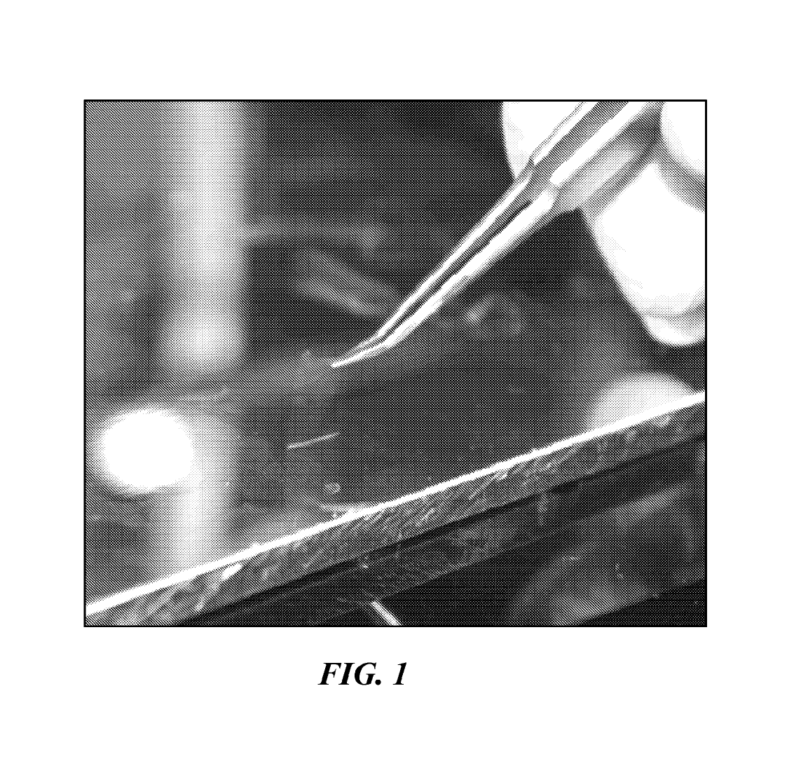

In some embodiments, a silk: glycerol blend is very flexible, yet robust. In some embodiments, thin layers printed from bio-ink compositions can easily be removed for example by peeling from a surface without breaking. FIG. 1 shows an about 2.5 μm to about 5 μm height silk film printed with 5.75 μL of 15% aqueous silk at an extrusion rate of 25 nL per 1.25 mm of travel in an area of 150 mm² without undesirable warping or stress localization between phases composing the printed layer to allow the production and handling of thin prints.

In some embodiments, porogens, for example, PMMA microspheres or PMMA rods are included in bio-ink compositions for printing. In some embodiments, a solid article includes such porogens. In some embodiments, porogens present in a solid article are dissolved away with a solvent, such as acetone. In some embodiments, with a porogen removed a solid article remains with pores within the printed constructs.

In some embodiments, bio-ink compositions for use in accordance with the present invention form printed articles of varying thickness when printed, deposited, and/or extruded. In some embodiments, bio-ink compositions for use in accordance with the present invention form printed articles of varying depth when printed, deposited, and/or extruded. In some embodiments, a single layer depth can vary from about 0.5 μm to about 100 μm. In some preferred embodiments, a single layer depth is about 5 μm to about 15 μm. In some embodiments, a single layer depth can be about 0.5 μm, about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, about 20 μm, about 21 μm, about 22 μm, about 23 μm, about 24 μm, about 25 μm, about 26 μm, about 27 μm, about 28 μm, about 29 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, about 110 μm, about 120 μm, about 130 μm, about 140 μm, about 150 μm, about 160 μm, about 170 μm, about 180 μm, about 190 μm, about 200 μm, about 225 μm, about 250 μm, about 275 μm, about 300 μm, about 325 μm, about 350 μm, about 375 μm, about 400 μm, about 425 μm, about 450 μm, about 475 μm, about 500 μm, about 600 μm, about 700 μm, about 800 μm, about 900 μm, about 1000 μm.

Complex Structures

In some embodiments, controlled printing, extruding or depositing bio-ink compositions for use in the practice of the present invention is advantageous for creating negative space. In some embodiments, replication of negative space within complex hollow structures allows printing of mechanical implant bodies, organ-like chambers, and scaffold vascularization. In some embodiments, bio-ink compositions dissolve away when desired, leaving hollow structures behind. In some embodiments, dissolvable inks contain linear polyols.

In some embodiments, bio-ink compositions useful for creating complex structures include one or more bio-ink compositions. In some embodiments, bio-ink compositions useful for creating complex structures include a pair of bio-ink compositions. In some embodiments, a pair of bio-ink compositions are useful for producing large scale, complex, irregular, and/or hollow 3D biocompatible, bioresorbable printing shapes. In some embodiments, a pair of bio-ink compositions include a sacrificial support material ink and permanent structural material ink.

In some embodiments, a bio based ink sacrificial support material ink further comprises an additive. In some embodiments, an additive suited for use in a sacrificial support material ink includes a hydrolyzed protein. In some embodiments, an additive suited for use in a sacrificial support material ink includes gelatin. In some embodiments, a bio based ink permanent structural material ink further comprises an additive. In some embodiments, an additive suited for use in a permanent structural material ink includes a polysaccharide. In some embodiments, an additive suited for use in a permanent structural material ink includes agar. In some embodiments, a specific pair for a process include a support material including 10% gelatin, 5% silk, 1% glycerol bulked bio-ink composition structural material is a 5% silk, 5% agar, 1% glycerol bulked bio-ink composition.

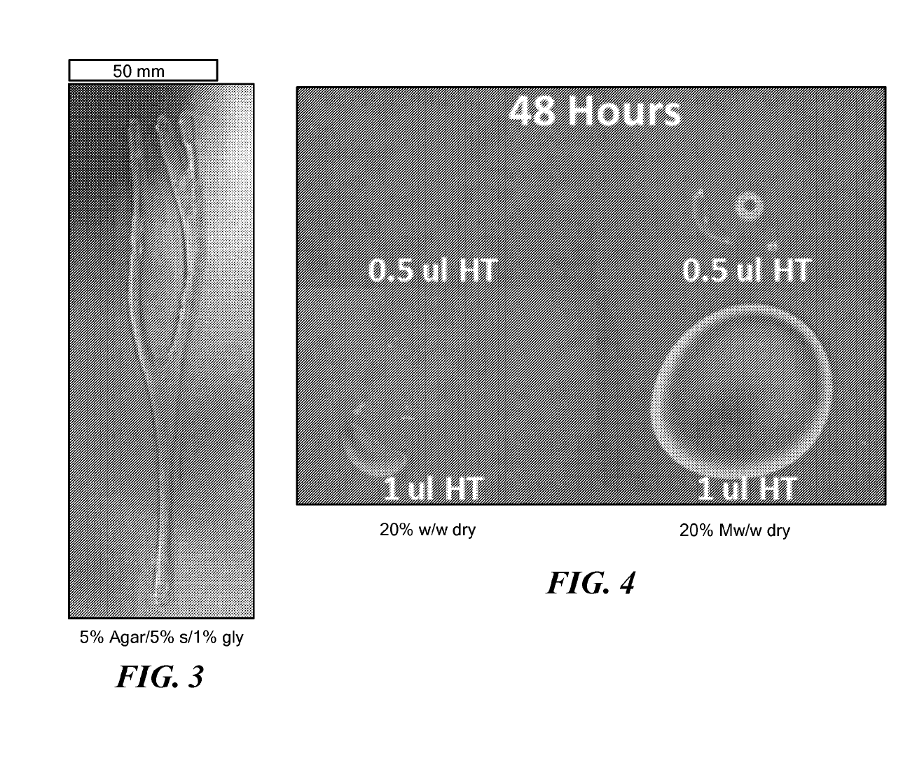

In some embodiments, the present invention includes a process of printing a desired shape capable of supporting overhangs and hollow chambers. In some embodiments, a process of printing a desired shape capable of supporting overhangs and hollow chambers includes a step of printing a construct. In some embodiments, a process of printing a desired shape capable of supporting overhangs and hollow chambers uses a permanent structural material and a sacrificial support material as disclosed herein for at least portions of a printed construct. In some embodiments, a process of printing a desired shape capable of supporting overhangs and hollow chambers includes a step of adding media or water to a permanent structural material and a sacrificial support material. In some embodiments, a process of printing a desired shape capable of supporting overhangs and hollow chambers includes a step of placing a construct in an incubator at 37° C. In some embodiments, a process of printing a desired shape capable of supporting overhangs and hollow chambers further includes steps of dissolving the support material and removing dissolved support material with media. In some embodiments, a process of printing a desired shape capable of supporting overhangs and hollow chambers results with a structural support material remaining true to shape. FIG. 3 shows a complex shape using such a formulation.

Crystalline Properties

Interestingly, silk fibroin has a different nature, being extruded from a living organism and changing its structure from globular to highly crystalline during such process. The scope of this work therefore included mimicking the natural silk fibroin extrusion process by inkjet printing regenerated silk solution, pioneering a new way to process this ancient material and providing unprecedented functions to fibroin-based biomaterials.

Prior to polypeptide and humectant bio-ink compositions of the present invention, conformational change was induced in polypeptides, including those of silk fibroin by any methods known in the art, including, but not limited to, alcohol immersion (e.g., ethanol, methanol), water annealing, shear stress, ultrasound (e.g., by sonication), pH reduction (e.g., pH titration and/or exposure to an electric field) and any combinations thereof. For example, the conformational change can be induced by one or more methods, including but not limited to, controlled slow drying (Lu et al., Biomacromolecules 2009, 10, 1032); water annealing (Jin et al., 15 Adv. Funct. Mats. 2005, 15, 1241; Hu et al., Biomacromolecules 2011, 12, 1686); stretching (Demura & Asakura, Biotech & Bioengin. 1989, 33, 598); compressing; solvent immersion, including methanol (Hofmann et al., J Control Release. 2006, 111, 219), ethanol (Miyairi et al., J. Fermen. Tech. 1978, 56, 303), glutaraldehyde (Acharya et al., Biotechnol J. 2008, 3, 226), and 1-ethyl-3-(3-dimethyl aminopropyl) carbodiimide (EDC) (Bayraktar et al., Eur J Pharm Biopharm. 2005, 60, 373); pH adjustment, e.g., pH titration and/or exposure to an electric field (see, e.g., U.S. Patent App. No. US2011/0171239); heat treatment; shear stress (see, e.g., International App. No.: WO 2011/005381), ultrasound, e.g., sonication (see, e.g., U.S. Patent Application Publication No. U.S. 2010/0178304 and International App. No. WO2008/150861); and any combinations thereof. Contents of all of the references listed above are incorporated herein by reference in their entireties. Alternative or additional methods known in the art that may induce an alteration in the conformation of certain structural proteins, such as silk fibroin, include shear stress. The shear stress can be applied, for example, by passing a structural protein composition through a needle. Other methods of inducing conformational changes include applying an electric field, applying pressure, and/or changing the salt concentration. Conformation of certain structural proteins, including silk fibroin, may be altered by water annealing. Without wishing to be bound by a theory, it is believed that physical temperature-controlled water vapor annealing (TCWVA) provides a simple and effective method to obtain refined control of the molecular structure of polypeptides. To illustrate a non-limiting example, in the case of silk fibroin, the relative degree of crystallinity can be controlled, ranging from a low beta-sheet content using conditions at 4° C. (α helix dominated silk I structure), to higher beta-sheet content of 60% crystallinity at 100° C. (β-sheet dominated silk II structure). Water or water vapor annealing is described, for example, in PCT application no. PCT/US2004/011199, filed Apr. 12, 2004 and no. PCT/US2005/020844, filed Jun. 13, 2005; and Jin et al., Adv. Funct. Mats. 2005, 15: 1241 and Hu et al., Biomacromolecules, 2011, 12(5): 1686-1696, contents of all of which are incorporated herein by reference in their entireties.

As described herein, in some embodiments, silk polypeptides exhibit an inherent self-assembly property and can stack with one another in crystalline layers. In some embodiments, various properties of such layers are determined, for example, by the degree of beta-sheet structure in the material, the degree of alpha-helical structure in the material, the degree of cross-linking between such beta sheets, the presence (or absence) of certain dopants or other materials. In some embodiments, one or more of these features is intentionally controlled or engineered to achieve particular characteristics of a silk matrix.

In some embodiments, a conformational change can be induced in such polypeptides or low molecular weight fragments thereof to control or tune the solubility of the protein-based structure printed on a substrate. Without wishing to be bound by a theory, the induced conformational change alters the crystallinity of the polypeptide, e.g., beta-sheet crystallinity.

In some embodiments, treatment time for inducing the conformational change using any of the above described methods may be any period to provide a desired degree of beta-sheet crystallinity content.

In some embodiments, a degree of crystallinity of polypeptides can be finely tuned and influences silk fibroin biological, physical, biochemical and mechanical properties. In addition, in some embodiments, the amino-acidic nature of silk fibroin brings a diversity of side chain chemistries that allows for the incorporation and stabilization of macromolecules useful in drug delivery applications or in providing cellular instructions. In particular, in some embodiments, dry silk fibroin with diverse degrees of crystallinity stabilizes vaccines and antibiotics. Silk fibroin is indeed considered a platform technology in biomaterials fabrication as its robustness and qualities bring the assets to add a large portfolio of distinct features (e.g. nanopatterning, biochemical functionalization) to the final construct.

In some embodiments, tuning, adjusting, and/or manipulating solubility or crystallinity of printed layer include, for example: selecting a specific polypeptide or selecting a specific humectant or a combination thereof

In some embodiments, solubility of a printed layer refers to a rate at which printed layers dissolve, degrade, denature, and/or decompose.

In some embodiments, 3D printed layers formed from bio-ink compositions as described herein are comprised of polypeptides, such as silk fibroin. In some embodiments, 3D bio-ink compositions comprising a polypeptide as described herein may contain a range of degrees/levels of crystallinity. In some embodiments, structures formed from provided bio-ink compositions may contain or comprise a crystalline content in a range of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, and about 100%.

In some embodiments, a degree of crystallinity of silk:glycerol prints is indicated by its beta sheet content. In some embodiments, beta sheet content is in a range of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, and about 100%. In some embodiments, beta sheet content of about less than 15% forms a soluble film. In some embodiments, beta sheet content of about greater than 60% forms a crystalline film.

In some embodiments, when blended, glycerol stabilizes an intermediate conformation of crystallized silk which produces a more flexible yet water insoluble film. In some embodiments, during printing, beta-sheet content increases to nearly 50% for insoluble films, whereas soluble films contain less than 15%. In some embodiments, increased beta-sheet content enables directly printing silk-based bio-inks into insoluble layers upon which additional layers can be subsequently printed. In some embodiments, there are no additional curing steps between printings of layers. In some embodiments, increased beta-sheet content enables fusion of non-thermoplastic elements to be conducted similar to conventional thermoplastic or UV curable printing polymers.

In some embodiments, a humectant may impart only partial solubility or partial crystallinity. Correspondingly, in comparison with glycerol, non-toxic polyols such as for example 1,3-propanediol and 1,2,6-hexanetriol and erythritol impart strikingly different solubility to printed biopolymer films, when used as additives. These qualities may be attributed to the varying degree of stabilization of water insoluble biopolymer intermediates, and subsequent effects on annealing due to the varying molecular size and hygroscopicity of the additive.

FIG. 4 shows (A) 25% w/w hexanetriol/silk ink printed into 0.5 microliter droplets demonstrate more solubility after 48 hours compared to (B) 25% Mw/w hexanetriol/silk. (C) 25% w/w hexanetriol/silk ink printed into 1 microliter droplets demonstrate more solubility after 48 hours compared to (D) 25% Mw/w hexanetriol/silk.

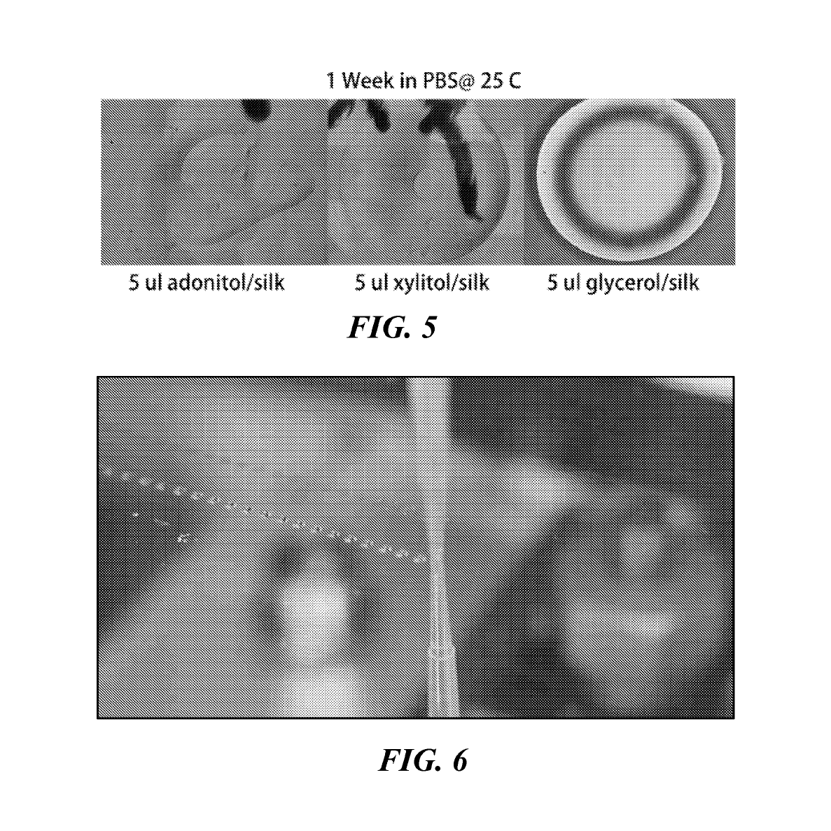

FIG. 5 shows (A) 25% w/w adonitol/silk ink printed into 5 microliter droplets demonstrate more solubility after 1 week compared to (B) 25% w/w xylitol/silk and (C) 25% w/w glycerol/silk.

In some embodiments, bio-ink composition may comprise multiple humectants.

In some embodiments, silk fibroin-based solutions may be formulated as “silk inks” for use in printing. Accordingly, the invention includes silk fibroin-based ink compositions and methods for manufacturing the same.

Alcohols such as methanol, ethanol, and isopropyl induce direct crystallization of the silk protein leaving behind insoluble aggregates but do not preserve original geometry of the print. Non-toxic polyols such as 1,3-propanediol and 1,2,6-hexanetriol and erythritol, as shown above, impart strikingly different solubility to printed silk films, when used as additives. These qualities may be attributed to the varying degree of stabilization of water insoluble silk intermediates, and subsequent effects on annealing due to the varying molecular size and hygroscopicity of the additive. This is largely due to additional beta-sheeting of the silk which is induced as water evaporates through the actively filming surface of the print. The volume of water which must pass through each square millimeter of the film surface increases proportionally to the radius of the print droplet, which in turn increases the time and degree of this induced beta-sheeting.

In some embodiments, solvents, for example, methanol can be printed onto the silk prints as an alternative method to create regions of increased crystallization.

In some embodiments, in addition to the time-dependent variability in surface profile, the strength of fusion between elements is dependent on the degree of curing allowed between subsequent prints. In some embodiments, optimal timing can be identified as the time corresponding to the maximal delamination strength. Conversely, in some embodiments, certain applications such as for example drug dispersion may benefit from the weakest delamination strength which will also be identified.

The lifetime (e.g., stability) of provided bio-ink compositions depends on the usage and the storage conditions. In some embodiments, storage in a refrigerator at 4 degree C. when finishing printing is recommended. In some embodiments, provided bio-ink compositions (with our without dopants) may be stored without refrigeration, such as at room temperature (typically between about 18° C. and about 26° C.) for an extended duration of time without significant loss of function. In some embodiments, provided bio-ink compositions (with our without dopants) may be stored at room temperature (typically between about 18° C. and about 26° C.) for an extended duration of time, such as at least for 1 week, at least for 2 weeks, at least for 3 weeks, at least for 4 weeks, at least for 6 weeks, at least for 2 months, at least for 3 months, at least for 4 months, at least for 5 months, at least for 6 months, at least for 9 months, at least for 12 months, at least for 15 months, at least for 18 months, and at least for 24 months, or longer, without significant loss of function. In some embodiments, provided bio-ink compositions (with our without dopants) may be stored at elevated temperature (between about 27° C. and about 40° C.) for at least part of the duration of storage, for an extended duration of time, such as at least for 1 week, at least for 2 weeks, at least for 3 weeks, at least for 4 weeks, at least for 6 weeks, at least for 2 months, at least for 3 months, at least for 4 months, at least for 5 months, at least for 6 months, at least for 9 months, at least for 12 months, at least for 15 months, at least for 18 months, and at least for 24 months, or longer, without significant loss of function.

Degradation Properties

In some embodiments, the present invention utilizes bio-ink compositions characterized in that they can be used to print articles with particular degradation properties.

To give but one example, in many embodiments, provided printing technologies utilize bio-inks to print biologically compatible articles, for example, for implantation into a bodyDegradability (e.g., bio-degradability) is often essential for such implantable articles.

Alternatively or additionally, the present invention encompasses the recognition that, in certain contexts, it may be desirable to prepare, provide, or utilize electronic articles or components that can degrade or be degraded. The present invention further encompasses the recognition that biocompatible, degradable (e.g., biodegradable) electronic articles or components are of particular interest.

Still further, the present invention encompasses the recognition that bio-ink compositions comprised of silk and/or silk fibroin polypeptides have a variety of desirable attributes, including degradability (e.g., biodegradability). Indeed, according to the present invention, one particularly desirable feature of silk-based materials is the fact that they can be programmably degradable. That is, as is known in the art, depending on particular features of a silk-based material (e.g., molecular weight of its silk components, degree of cross-linking, degree of crystallization, degree beta-sheet content or combinations thereof) can be controlled to degrade at certain rates. Degradability of (and also controlled release from) a silk-based material has been studied; a variety of relevant information has been published (see, for example, WO 2004/080346, WO 2005/012606, WO 2005/123114, WO 2007/016524, WO 2008/150861, WO 2008/118133, each of which is hereby incorporated by reference in its entirety herein).

In some embodiments, printed 3D articles in accordance with the present invention comprise bio-ink compositions that release agents over time such as those agents above described. In some embodiments, a bio-ink compositions are associated with agents. In some embodiments, a printed layer of a silk scaffold is associated with agents. In some embodiments, a bio-ink composition and/or printed layer is associated with multiple agents. In some embodiments, composite printed layers degrade, decompose, and/or delaminate releasing agent(s) at a site. In some embodiments, multiple agents are released in a cascade, for example, multiple agents may be released in a cascade of growth factor mimicking a natural bone repair and regeneration process.

Control of silk material production methods as well as various forms of silk-based materials can generate silk compositions with known degradation properties. For example, using various silk fibroin materials (e.g., microspheres of approximately 2 μm in diameter, silk film, silk hydrogels) entrapped agents such as therapeutics can be loaded in active form, which is then released in a controlled fashion, e.g., over the course of minutes, hours, days, weeks to months. It has been shown that layered silk fibroin coatings can be used to coat substrates of any material, shape and size, which then can be used to entrap molecules for controlled release, e.g., 2-90 days.

Interlayer Interactions

In some embodiments, printed articles comprising one or more printed layers of a bio-ink composition are characterized by an interlayer interactions or a degree of fusion between layers. In some embodiments, interlayer interactions contribute at least to some extent to a geometry of a printed article. In some embodiments, interlayer interactions contribute at least to some extent to the properties of a printed article. In some embodiments, properties include for example stress, strain. etc.

### Methods of Forming Printed Articles

Bio-printing or bio-based printing is a process of printing, depositing, and/or extruding biological materials to engineer 2D and 3D structures capable of comprising biomaterials and living structures. In 2004, the first international workshop on the subject defined bioplotting or bioprinting as “the use of material transfer processes for patterning and assembling biologically relevant materials—molecules, cells, tissues, and biodegradable biomaterials—with a prescribed organization to accomplish one or more biological functions.” (See Mironov, V. et al., Bioprinting: A Beginning, 12 Tissue Engineering, 631-634 (2006). The ability to design tailored implant and scaffold geometries using 3D patient scans and computer-aided design currently exist. However, in order to transcend from the virtual to the real, patient-specific designs require the development of accurate high-resolution fabrication techniques.

As described herein, the present invention provides technologies for the production of 2D and 3D articles by printing a bio-ink composition. In some embodiments, a bio-ink composition having a ratio of a polypeptide (e.g. silk fibroin) to a humectant (e.g. glycerol) may be about 20 to 1, about 15 to 1, about 10 to 1, about 5 to 1, about 2 to 1, or about 1 to 1. In some embodiments, a ratio of glycerol to silk can be modulated to influence the degree of imparted insolubility.

In some embodiments, 3D structures of bio-ink composition are formed from composition produced using a blend of glycerol and silk. In some embodiments, 3D structures of bio-ink composition are formed from composition produced using about 2% w/v to about 25% w/v of the ink and the humectant comprises about 2% w/v to about 30% w/v of the ink. In some embodiments, 3D structures of bio-ink composition are formed from composition produced using 5 μm to 1500 μm structures have been fabricated using 5% w/v glycerol with 15% w/v silk processed using 30 minutes of heat-induced molecular weight reduction.

In some embodiments, a printed article by extruding a bio-ink composition.

In some embodiments, a printed article forms when a bio-ink composition cures. In some embodiments, a printed article forms by an active curing process. In some embodiments, an active cure includes application of a dose of electromagnetic radiation. In some embodiments, an active cure includes application of heat. In some embodiments, an active cure includes a chemically induced curing. In some embodiments, a printed article forms by an passive curing process.

In some embodiments, bio-ink compositions self-cure. In some embodiments, bio-inks comprising a polypeptide (e.g. silk fibroin as described above) and a humectant (e.g. glycerol, as described above) self-cure. Glycerol is a simple metabolizable non-toxic sugar alcohol, ubiquitous in food and pharmaceutical industries. Without wishing to be bound by a theory, mechanistically it is believed that when blended with a polypeptide, such as a silk polypeptide, glycerol stabilizes an intermediate conformation of crystallized silk. Glycerol acts to replace water in a silk fibroin chain hydration, resulting in the initial stabilization of helical structures in the films, as opposed to random coil or β-sheet structures, which produces a more flexible yet water insoluble film. (See Lu, S. et al. Insoluble and Flexible Silk Films Containing Glycerol, 11 Biomacromolecules, 143-150 (2010), which is hereby incorporated by reference in its entirety herein). Due to the ability of glycerol to stabilize insoluble conformations of silk, silk: glycerol blends are attractive for use in the development of a non-toxic bio-ink composition base. (See id.) In some embodiments, a silk and glycerol blend enables directly printing silk-based bio-ink compositions into insoluble layers upon which additional layers can be subsequently printed. In some embodiments, there is no need for additional curing steps between printings of layers. In some embodiments, this allows fusion of non-thermoplastic elements to be conducted similar to conventional thermoplastic or UV curable printing polymers.

In addition to or as an alternative to glycerol, in some embodiments, other humectants (for example, those from the sugar alcohols/sugar polyols category) may produce similar self-curing results. In some embodiments, linear polyols are blended with silk into 25% w/w (dry) ratios and then printed into 1-microliter droplets. Table 2 provides a summary of the solubility of films produced from various silk/linear polyol blends.

TABLE 2

| | Additive | | | | | | Blend Ratio | Solubility Time [min] | | |
|---|---|---|---|---|---|---|---|---|---|---|
| | Hazard | | C— | —OH | Density | Molar Mass | silk:additive | Droplet Size [μm] | | |
| Name | NFPA 704 | Structure | # | # | [g/cm³] | [g/mol] | dry [w/w] | 0.5 | 1 | 5 |

Solubility of silk:polyol blends [in PBS @ 20° C.]

| | | | | | | | | | | |
|---|---|---|---|---|---|---|---|---|---|---|
| None | — | — | — | — | — | — | 100/0 | 1 | 1 | 1 |
| Glycerol | 0 | Linear | 3 | 3 | 1.26 | 92.09 | 75/25 | NS | NS | NS |
| Methanol | 1 | Linear | 1 | 1 | 0.79 | 32.04 | 75/25 | 1 | 1 | 1 |
| Ethanol | 1 | Linear | 2 | 1 | 0.79 | 46.07 | 75/25 | 1 | 1 | 1 |
| Ethylene glycol | 2 | Linear | 2 | 2 | 1.11 | 62.07 | 75/25 | NS | NS | NS |
| Isopropyl | 1 | Linear | 3 | 1 | 0.79 | 60.10 | 75/25 | 1 | 1 | 1 |
| 1,3-Propanediol | 0 | Linear | 3 | 2 | 1.06 | 76.09 | 75/25 | NS | NS | NS |
| Butane | 1 | Linear | 4 | 0 | 0.00 | 58.12 | 75/25 | 1 | 1 | 1 |
| 1,4-Botanediol | 1 | Linear | 4 | 2 | 1.02 | 90.12 | 75/25 | NS | NS | NS |
| Diethylene glycol | 1 | Linear | 4 | 2 | 1.12 | 106.12 | 75/25 | NS | NS | NS |
| −+ Butanetriol | 2 | Linear | 4 | 3 | 1.19 | 106.12 | 75/25 | NS | NS | NS |
| −− Butanetriol | 2 | Linear | 4 | 3 | 1.19 | 106.12 | 75/25 | NS | NS | NS |
| Erythritol | 0 | Linear | 4 | 4 | 1.45 | 122.12 | 75/25 | 1 | 1 | NS |
| D,L Threitol | 2 | Linear | 4 | 4 | 1.43 | 122.12 | 75/25 | 1 | 1 | NS |
| 1,5-Pentanediol | 2 | Linear | 5 | 2 | 0.99 | 104.15 | 75/25 | NS | NS | NS |
| 1,2-Pentanediol | 2 | Linear | 5 | 2 | 0.99 | 104.15 | 75/25 | NS | NS | NS |
| Adonitol | 0 | Linear | 5 | 5 | 1.52 | 152.15 | 75/25 | P | P | P |
| Xylitol | 0 | Linear | 5 | 5 | 1.52 | 152.15 | 75/25 | P | P | P |
| Hexane | 2 | Linear | 6 | 0 | 0.65 | 86.18 | 75/25 | 1 | 1 | 5 |
| 1,2,6-Hexanetriol | 0 | Linear | 6 | 3 | 1.11 | 134.17 | 75/25 | 1 | 1 | NS |
| Sorbitol | 0 | Linear | 6 | 6 | 1.49 | 182.17 | 75/25 | 1 | 1 | 1 |
| Mannitol | 0 | Linear | 6 | 6 | 1.49 | 182.17 | 75/25 | 1 | 1 | 1 |
| 1,2-Octanediol | 1 | Linear | 8 | 2 | 0.91 | 146.23 | 75/25 | P | P | P |
| Galactose | 0 | Ring | 6 | 5 | 1.72 | 180.16 | 75/25 | 1 | 1 | 1 |
| Trehalose | 0 | Ring | 12 | 8 | 1.58 | 342.30 | 75/25 | 1 | 1 | 1 |

Solubility of silk:polyol blends normalized for molar weight[in PBS @ 20° C.]

| | | | | | | | | | | |
|---|---|---|---|---|---|---|---|---|---|---|
| None | — | — | — | — | — | — | 100/0 | 1 | 1 | 1 |
| Glycerol | 0 | Linear | 3 | 3 | 1.26 | 92.09 | 75/25 | NS | NS | NS |
| 1,2,6-Hexanetriol | 0 | Linear | 6 | 3 | 1.11 | 134.17 | 75/25 | 1 | 1 | NS |
| 1,2,6-Hexanetriol | 0 | Linear | 6 | 3 | 1.11 | 134.17 | 64/36 | 1 | NS | NS |

*Numerical values indicate time to completely dissolve the printed droplet without leaving detectable remnants
NS**Not Soluble
P***Partially soluble; obvious reduction in volume; thin film residue remains
NFPA 704—standard safety data sheet chemical hazard identifier

In some embodiments, a printed article cures in accordance with a drying time. In some embodiments, a short drying time occurs between printing of subsequent layers. In some embodiments, a short drying time is in a range between about 0.1 seconds and about 600 seconds. In some embodiments, drying time is dependent on a layer thickness. In some embodiments, drying time is dependent on a volume of ink. In some embodiments, drying time is dependent on environmental factors. In some embodiments, environmental factors include, for example, temperature and/or humidity. In some embodiments, selecting drying time occurs when layer thickness, temperature, humidity, or combinations thereof are controlled during deposition.

FIG. 2 shows that select polyols, in particular linear polyols in addition to glycerol, impart various levels of solubility to subsequent silk blended films, yet add little to no toxicity. Carbon and hydroxyl count increase from left to right. Persistence or dissolution of droplet blend in PBS at 20° C. from 1 min to 48 hours is displayed.

In some embodiments, as potential applications broaden, the degree of desired solubility may shift beyond the range achievable with glycerol blends. In some embodiments, alcohols such as methanol, ethanol, and isopropyl induce direct crystallization of the silk protein leaving behind insoluble aggregates but do not preserve the original geometry of the print.

In some embodiments, bio-ink compositions as described herein do not require damaging processing steps, as such cellularization or drug deposition can be performed in parallel with structural printing. In some embodiments, bio-ink compositions comprising a blend of a polypeptide and a humectant are specifically designed to allow real-time incorporation of temperature or UV sensitive biologicals such as pharmaceuticals, growth factors, or cells as additives. In some embodiments, bio-ink compositions comprising a blend of a polypeptide and a humectant enable single-step printing of transitional and 3D encapsulated elements, which as described above cannot be fabricated using other methods. Formation of printed articles as described herein exploits evaporation-induced buckling of silk depositions which, when blended with certain non-toxic additives, cure to crystallized structural prints. This phenomenon allows bypassing of deleterious curing mechanisms which are prevalent in alternative 3D rapid prototyping techniques as above described.

In some embodiments, application of multiple layers of ink are applied by serially printing individual layers of a bio-ink composition on a substrate. In some embodiments, provided 3D printing technologies involve application of multiple layers of ink (e.g., bio-ink composition) wherein individual layers dry to a crystallized state during printing. In some embodiments, provided 3D printing technologies involve application of multiple layers of ink (e.g., bio-ink composition) wherein individual layers dry to a crystallized state before printing of subsequent layers. In some embodiments, layers of ink are printed substantially concurrent with prior layers so that upon completion of a single pass whereby a bio-ink composition layer has been deposited, additional layers of ink may readily deposit without solubilizing prior layers.

In some embodiments, 3D structures formed from a bio-ink composition comprising a humectant added to a polypeptide are robust and generate impressive mechanical strength comparable to traditional regenerated silk fibroin films.

In some embodiments, 3D structures formed from a bio-ink composition comprising a blend of a humectant added to a polypeptide for use in accordance with the present invention have sharp angles and clean edges when immersed in solvent, transferred to simulated physiological environments, or completely submersed in water/PBS are capable of maintaining their crystalline structure.

In some embodiments, fused or interconnected structures of varying geometry are formed when such stabilized 2D and/or 3D polypeptide structures are overlaid. In some embodiments, overlaid structures are formed from printing, extruding, and/or depositing bio-ink compositions. In some embodiments, geometries of overlaid structures comprise structural regions of a cured printed bio-ink composition and regions wherein agents or additives have been incorporated, such as for example cellularized regions that include cells as an agent. To date, curing printed bio-ink compositions to form structurally robust 3D printed articles has not been possible. While curing mechanisms associated with cell-printers are less biologically damaging, 3D prints fabricated from such printers, printed articles produced from such methods have been shown to lack mechanical and/or cohesive strength.

A Robotic Deposition System for Printing 2D and 3D Structures with Bio-Ink Compositions

Among other things, the present application provides a programmable bio-ink composition printing system (“bio-ink composition printer”/“bio-printer”/“bio-plotter”/“robotic deposition system”) for design and fabrication of 2D and 3D high-resolution, cytocompatible printed layers formed from bio-ink compositions as disclosed herein. In some embodiments, bio-ink compositions, for example form stabilized 2D and 3D polypeptide structures when printed using printing apparatus as described herein. In some embodiments, fused or interconnected structures of varying geometry are formed when a bio-ink compositions are overlaid printed article. In some embodiments, overlaid structures are formed from printing, extruding, and/or depositing bio-ink compositions.

In some embodiments, a robotic deposition system of the present invention comprises hardware and programmable schemes to modulate deposition of bio-ink composition. In some embodiments, high-resolution replication of micro-scale CAD features of such programmed schemes is possible using robotic deposition systems as described herein. In some embodiments, robotic deposition systems as described herein are capable of achieving 3D prints with such high resolution features and are capable of achieving 3D prints assembled from a high number of cross-sectional layers with high resolution features. In some embodiments, printed features demonstrate macro-scale geometry and structural strength when printed using robotic deposition systems as described herein.

In a stark departure from traditional fused deposition modelling, an approach to printing as disclosed with the present invention uses liquid or gel “inks,” such as bio-ink compositions as described herein. In some embodiments, evaporation mechanisms enables curing of bio-ink compositions without additional toxic processes. As detailed herein, such an approach largely avoids the above described problems traditionally known in the art. However, in some embodiments, when compared to thermoplastic cords or cell pastes, bio-ink compositions for use in accordance with the present invention contain a larger fraction of water as solvent. In some embodiments, a larger fraction of water as solvent increases a number printing variables, thereby demanding more sophisticated printer control. Printed element deposited with a larger fraction of water may exhibit some volume buckling as solvent is evaporated. Such buckling generates large changes to a printed surface profile thereby complicating printing of subsequent layers.

In some embodiments, to compensate, printer hardware as described herein is capable of high-resolution positioning and deposition minimizes complications associated with printing of subsequent layers. In some embodiments, smaller volume depositions result in shorter buckling of printed profile height reducing variance in distance between an extruder tip and printed surface before and after curing. In some embodiments, reduced printing distance and shorter buckling generates higher resolution prints. In some embodiments, reducing a volume of printing ink extruded during a period generates higher resolution prints. In some embodiments, hardware, programming, and deposition schemes described herein create precise spatial control, patterns of discreet printed elements, and anisotropic gradients thereby generating bio-prints with high resolution fusions of independent elements.

### Printing Surfaces

A variety of substrates may be suitable for use in 3D printing of a bio-ink composition described herein. Such printable substrates using bio-ink compositions are limitless, depending on the available printers. Non-limiting examples of useful substrates include, but are not limited to: papers, polyimide, polyethylene, natural fabric, synthetic fabric, metals, liquid crystal polymer, palladium, glass and other insulators, silicon and other semiconductors, metals, cloth textiles and fabrics, plastics, biological substrates, such as cells and tissues, protein- or biopolymer-based substrates (e.g., agarose, collagen, gelatin, etc.), and any combinations thereof

In some embodiments, provided bio-ink compositions can be printed on substrates that generally are of a flexible material, such as a flexible polymer film or paper, such as wax paper or non-wax substrates. In some embodiments, suitable substrates include releasable substrates, such as a label release grade or other polymer coated paper, as is known in the art, see for example U.S. Pat. No. 6,939,576, which is hereby incorporated by reference in its entirety herein. Such substrate also can be or include a non-silicone release layer. Such substrate also can be a plastic or polymer film, such as anyone of an acrylic-based film, a polyamide-based film, a polyester-based film, a polyolefm-based film such as polyethylene and polypropylene, a polyethylene naphthylene-based film, a polyethylene terephthalate-based film, a polyurethane-based film or a PVC-based film, or a combination thereof

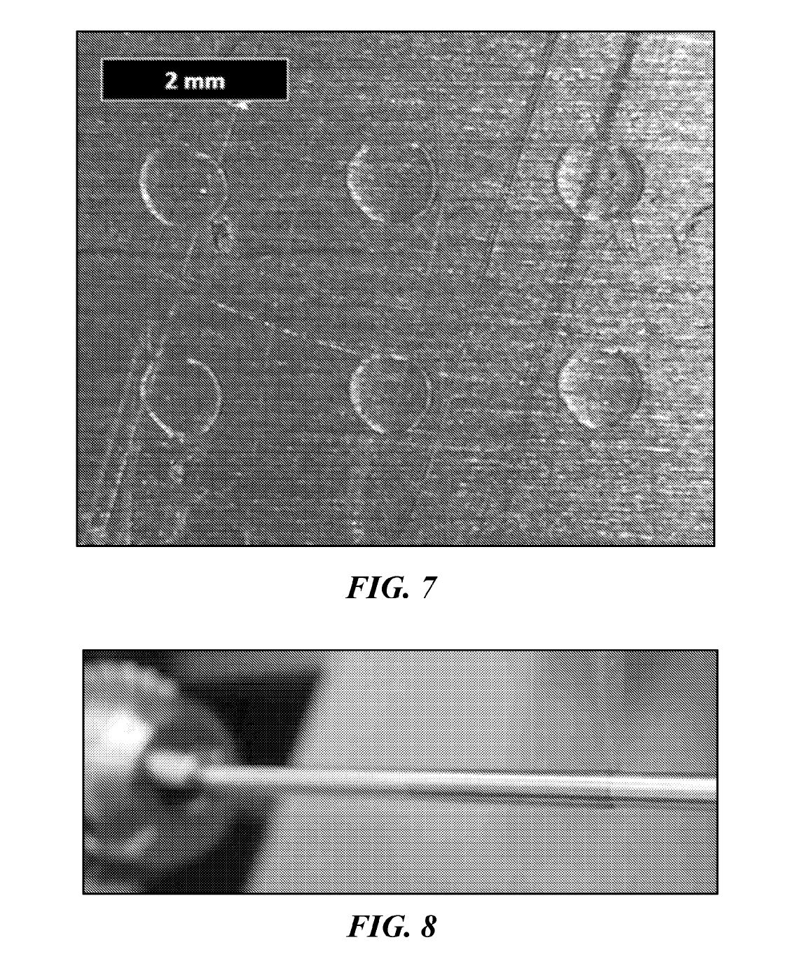

In some embodiments, printable surfaces include highly polished surfaces and uneven surfaces. FIG. 6 shows dots printed on a mirror polished aluminum substrate. In contrast, FIG. 7 shows dots printed on a visibly rough aluminum substrate.

In some embodiments, printable surfaces include rotatable substrates. In some embodiments, rotatable substrates including tubing. FIG. 8 shows a printed layer on the outside diameter of a tube. In some embodiments, a rotatable surface is or a rotatable surface is mounted to a rotatable chuck.

### 3D Biopolymer Ink Printers

Apparatus and methods for creating polypeptide-based or protein-based prints or arrays are known in the art, including for example pen spotting, soft lithography, photolithography, and drop-on-demand inkjet printing. Bio-printing equipment capable of being used by those skilled in the art is described for example in the following publications which are hereby incorporated by reference in their entirety herein: Kaji K. et al., The Mechanism of Sperm-Oocyte Fusion in Mammals, 127 Reproduction 423-29 (2004); Whitesides G. M. et al., Soft Lithography in Biology and Biochemistry, 3 Annual Review Biomedical Engineering, 335-373 (2001); Falconnet D. et al., A Novel Approach to Produce Protein Nanopatterns by Combining Nanoimprint Lithography and Self-Assembly, 4 Nano Letters, 1909-1914 (2004); Ito Y. et al., Micropatterned Immobilization of Epidermal Growth Factor to Regulate Cell Function, 9 Bioconjugate Chemistry, 277-282 (1998); Chen G. et al., Gradient Micropattern Immobilization of EGF to Investigate the Effect of Artificial Juxtacrine Stimulation, 22 Biomaterials, 2453-2457 (2001); Zaugg F. et al., Drop-on-Demand Printing of Protein Biochip Arrays, MRS Bulletin, 837-842 (2003); Watanabe K. et al., Growth Factor Array Fabrication Using a Color Ink Jet Printer, 20 Zoological Science, 429-434 (2003); Boland T, Application of Inkjet Printing to Tissue Engineering, 1 J. Biotechnology, 910-7 (2006); Campbell P. G. et al., Tissue Engineering with the Aid of Inkjet Printers, 7 Expert Opinion on Biological, 1123-7 (2007); Nakamura M. et al., Biocompatible Inkjet Printing Technique for Designed Seeding of Individual Living Cells, 11 Tissue Engineering, 1658-66 (2005); Nishiyama Y. et al., Development of a Three-Dimensional Bioprinter: Construction of Cell Supporting Structures using Hydrogel and State-of-the-Art Inkjet Technology, 131 J. Biomechanical Engineering, 035001 (2009); Phillippi J. A. et al., Microenvironments Engineered by Inkjet Bioprinting Spatially Direct Adult Stem Cells Toward Muscle- and Bone-Like Subpopulations, 26 Stem Cells, 127-34 (2008); Saunders R. E. et al., Delivery of Human Fibroblast Cells by Piezoelectric Drop-on-Demand Inkjet Printing, 29 Biomaterials, 193-203 (2008); Xu T. et al., Viability and Electrophysiology of Neural Cell Structures Generated by the Inkjet Printing Method, 27 Biomaterials, 3580-8 (2006); Xu T. et al., Inkjet Printing of Viable Mammalian Cells, 26 Biomaterials, 93-9 (2005); Yamazoe H. et al., Cell Micropatterning on an Albumin-Based Substrate using an Inkjet Printing Technique, 91 J. Biomedical Materials Research A, 1202-9 (2009); Smith C. M., Characterizing Environmental Factors that Impact the Viability of Tissue-Engineered Constructs Fabricated by a Direct-Write Bioassembly Tool, 13 Tissue Engineering, 373-83 (2007); Smith C. M. et al., Three-Dimensional Bioassembly Tool for Generating Viable Tissue-Engineered Constructs, 10 Tissue Engineering, 1566-76 (2004).

Generally, 3D printers are capable of creating objects and/or structures in three dimensions through computer assisted 3D design and fabrication. Computer-assisted 3D printers to be used in fabrication of computer-designed objects progressively deposit material by additive manufacturing processes. Typically, 3D printers serially print by successively layering of materials.

In some embodiments, high resolution positioning is accomplished by employing stepper motors to accurately drive linear motion via translation screws. In some embodiments, multi-motor stepper controlled robotics facilitate reproducibility and throughput with a sub-nanometer level of control of print-head positioning and extrusion. Multi-motor stepper controlled robotics having reproducibility and throughput with a sub-nanometer level of control of print-head positioning and extrusion are known to those skilled in the art, for example in S. C. Jordan et al., 10 Current Pharmaceutical Biotechnology, 515 (2009); J. Otsuka, 3 Nanotechnology (1992), which are incorporated herein by reference. Precise positioning control is possible for more than two axes (3D printing). In some embodiments, ample resolution for X-Y positioning is available. Moreover, in some embodiments, high resolution is also available for Z positioning for printing of subsequent layers. In some embodiments, extrusions will be driven using stepper motors and leadscrews.

In some embodiments, printer hardware as described herein is capable of high-resolution for positioning and depositing of bio-ink compositions, thereby minimizing mechanical obstacles associated with printing of subsequent layers using bio-ink compositions as described herein. In some embodiments, a programmable bio-ink composition printing system for use in fabrication of 2D and 3D high-resolution, cytocompatible biopolymer prints comprises hardware and programmable schemes to modulate deposition of bio-ink composition. In some embodiments, high-resolution replication of micro-scale CAD features of such programmable schemes is possible using robotic deposition systems as described herein. In some embodiments, robotic deposition systems as described herein are capable of achieving 3D prints with high slice number. In some embodiments, printed features demonstrate macro-scale geometry and structural strength when printed using robotic deposition systems as described herein.

In some embodiments, a printing systems possessing a modular design provides a platform that supports at least one extruder and planar and tubular printing surfaces. In some embodiments, extrusions will be driven using stepper motors and leadscrews. In some embodiments, leadscrew positioning is driven with 2-phase unipolar stepper motors capable of discrete 1.8° step angles. In some embodiments, stepper motors are driven by ⅛ microstep drivers to further smooth discrete leadscrew rotations. In some embodiments, leadscrews generate 1.27 mm of linear movement per rotation. In some embodiments, leadscrews with rolled ¼″-20 threads were mated to graphite leadnuts to improve smoothness and reduce required low end torque which translates into increased accuracy. In some embodiments, a minimal increment of programmable linear movement is between about 0.05 μm and about 1.0 mm. In some embodiments, a minimal increment of programmable linear movement is between about 0.1 μm and about 100 μm. In some embodiments, a minimal increment of programmable linear movement is between about 0.2 μm and about 50 μm. In some embodiments, a minimal increment of programmable linear movement is between about 0.5 μm and about 25 μm. In some embodiments, a minimal increment of programmable linear movement is between about 0.6 μm and about 15 μm. In some embodiments, a minimal increment of programmable linear movement is between about 0.7 μm and about 10 μm. In some embodiments, a minimal increment of programmable linear movement is between about 0.8 μm and about 6.35 μm. In some embodiments, a minimal increment of programmable linear movement is dependent on an ability of applied microstep current to overcome system friction. In some embodiments, extrusion is driven using stepper motors and leadscrews of having same specifications. In some embodiments, extrusion increments are dependent on syringe barrel diameter.

In some embodiments, control of mechanical and solubility properties is a function of selected additives and blend ratios. In some embodiments, mechanical and solubility properties may be optimized through selection of additives and blend ratios. In some embodiments, a selection of additives and blend ratios generates varying printed filament size. In some embodiments, print properties and printed filament size varies with print element volume. In some embodiments, print element volume varies due to additional beta-sheeting of the biopolymer (e.g., silk) which is induced as water evaporates through the actively filming surface of the print. In some embodiments, a volume of water which must pass through each square millimeter of film surface increases proportionally to a radius of a print droplet, which in turn increases a time and degree of induced beta-sheeting. In some embodiments, printed layers are attainable when printing depositing or extruding smaller print volumes.

In some embodiments, a smallest volume of ink which can be deposited is about 0.1 nL, 0.5 nL, about 1 nL, about 1.5 nL, about 2 nL, about 2.5 nL, about 3 nL, about 4 nL, about 5 nL, about 6 nL, about 7 nL, about 8 nL, about 9 nL, about 10 nL, about 11 nL, about 12 nL, about 13 nL, about 14 nL, about 15 nL, about 20 nL, about 25 nL, about 30 nL, about 35 nL, about 40 nL, about 45 nL, about 50 nL, about 55 nL, about 60 nL, about 65 nL, about 70 nL, about 75 nL, about 80 nL, about 85 nL, about 90 nL, about 95 nL, about 100 nL, about 105 nL, about 110 nL, about 115 nL, about 120 nL, about 125 nL, about 150 nL, about 175 nL, about 200 nL, about 250 nL, about 500 nL, or at least about 1000 nL.

In some embodiments, a minimal increment of programmable linear movement is increments of 0.8 μm to 6.35 μm, as such a smallest volume of ink which can be deposited from a standard 5 mm diameter syringe is 1 nL to 125 nL.

In some embodiments, bio-ink compositions can be stored in standard syringes as the system will be designed to use syringes as ink and gel reservoirs. In some embodiments, to enable real-time modulation of blends for the printing of gradients, a blending nozzle with multiple inlets will be used. In some embodiments, multiple influent lines will be used to supply bio-ink compositions of varying blends with additional extruders.

In some embodiments, computer numerical control of stepper motors will be performed for example using an Arduino microcontroller. In some embodiments, computer numerically controlled printing schemes will be programmed to deposit soluble and/or structural bio-ink composition elements described herein. In some embodiments, instructions can be accomplished via manual programming written in C++ or by generating interpreted programming. In some embodiments, interpreted programming can be generated in steps after desired geometry is designed using CAD. In some embodiments, desired geometry will be converted into layer stacks using a slicing algorithm.

In some embodiments, a design must be converted into packets of discrete commands to turn a virtual design into machine instructions which are then translated into precise stepper movements. In some embodiments, G programming language is widely used as a numerical control programming language for creating such machine instructions for computer-aided engineering in automation. Converting designs into precise stepper movements can be accomplished by those in the art, see for example K. H. Jeon et al., Proc. 30th Int. Symp. Autom. Robot. Constr. Min., International Association For Automation And Robotics In Construction, Montreal, Canada, 2013, pp. 1359-1365; and S. K. Sinha, 2 Int. J. Eng. Sci. Technol., 7616 (2010), which are incorporated herein by reference.

In some embodiments, each layer in the stack will be converted G-code positioning commands. In some embodiments, G-code will next require manual programming edits to be compatible with the custom printing system. In some embodiments, after editing and simulating the programmed run with host-controller software, it may be possible to interpret the code directly for use with the Arduino microcontroller.

In some embodiments, depending on the design of the robotics, each precise stepper movement will be translated into a discrete nano- to pico-level extrusion or change in position by the print-head. In some embodiments, the precision of the positioning and extruder response to these commands is dependent on the limitations of the robotics. In some embodiments, further print precision may depend on nozzle geometry, extruder clearance, rate consistency, and surface tension. In some embodiments, fusion of print elements and isolation of print elements can also be modulated by print spacing. In some embodiments, structural configurations are formed when bio-ink compositions are printed, deposited, and/or extruded in the form of lines or microdroplets.

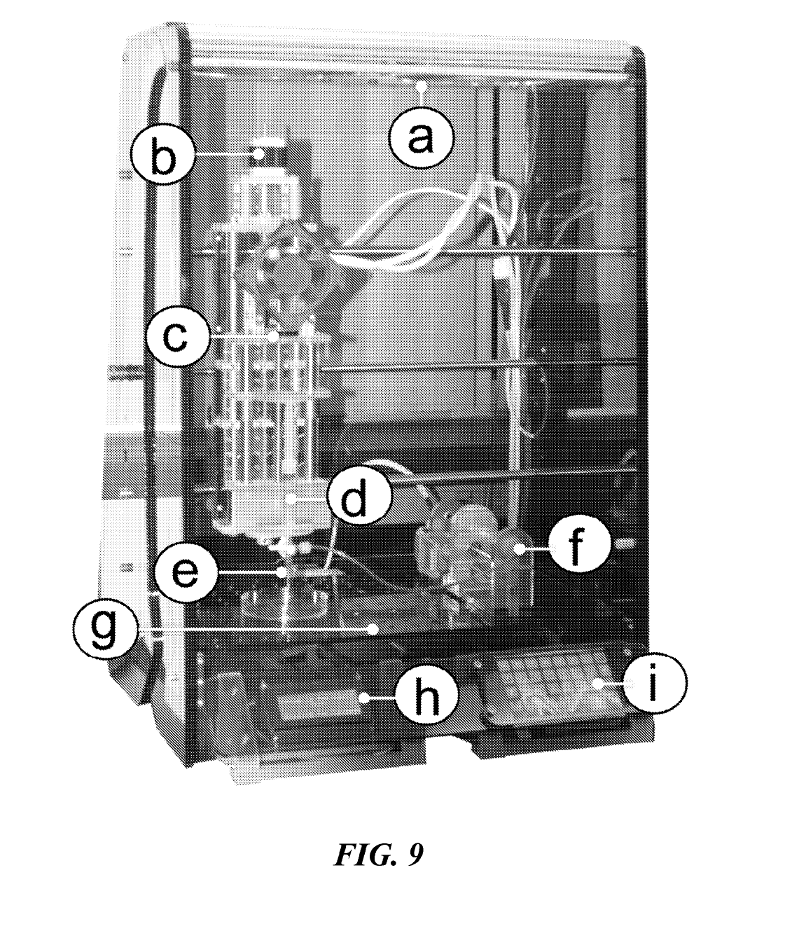

FIG. 9 shows a 3D bio-ink composition printer 100 as part of a 3D printing platform. A functional hybrid printing system, as shown in FIG. 9 has been fabricated. In some embodiments, a functional hybrid printing system is a programmable bio-ink composition printing system for design and fabrication of 2D and 3D high-resolution, cytocompatible biopolymer prints is disclosed herein. In some embodiments, cytocompatible biopolymer prints include for example polypeptide based structures. In some embodiments, prints include agents and/or additives as disclosed herein. In some embodiments, prints are fabricated from bio-ink compositions as disclosed herein.

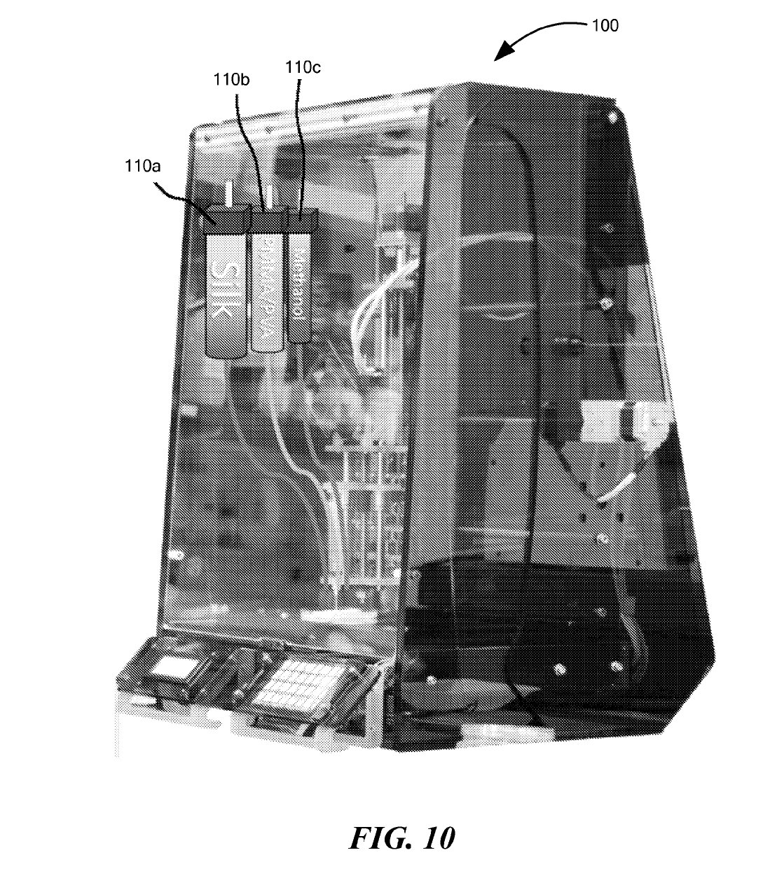

FIG. 9 shows an exemplary silk bio-printer in accordance with the present invention. FIG. 9 shows labels indicating the following typical components: (a) dual exhaust fans; (b) z-axis stepper; (c) extrusion stepper; (d) syringe ink cartridge; (e) dual ink extruder; (f) secondary extruder; (g) 60 cm×40 cm XY dual axis printing stage; (h) LCD HMI menu display; and (i) custom HMI for loading of manually programmed commands. FIG. 10 shows multiple microstepped extruders to modulate ink blend during printing to produce gradients or anisotropic properties.

In some embodiments, 3D printer for bio-ink composition printing includes a three-axis positioning system, such as an x, y, z stage configured for movement of printing heads. In some embodiments, a 3D printer for bio-ink composition printing includes a computing system configured to design and/or execute computer script. In some embodiments, a bio-ink composition printer, also includes for example, an extruder configured to displace a bio-ink composition. In some embodiments, a 3D printer for bio-ink composition printing applications may include a bio-ink composition cartridge. In some embodiments, a bio-ink composition cartridge is configured to be filled with multicellular building blocks that can be spheroidal or cylindrical depending on the method of preparation.

Extruder Tip Design

In some embodiments, a 3D printing system includes a dual ink extruder. In some embodiments, a 3D printing system includes a multi-ink extruder. In some embodiments, a 3D printing system including more than one extruder may also include an aspirator line. In some embodiments, an aspirator is useful for reducing printing time.

In some embodiments, a 3D printing system including more than one extruder tip uses a mixing chamber. In some embodiments, an extruder tip is a microfluidic tip. In some embodiments, a microfluidic tip uses microstepper motors for precision control and release of bio-ink compositions. In some embodiments, a mixing chamber of a 3D printing system including more than one extruder tip will be flooded with a first ink, ink-A when switching to a second ink, ink-B, In some embodiments, switching from ink-A to a second ink, ink-B, will cause lag in printing time. In some embodiments, a lag time associated with switching from ink-A to ink-B occurs as a printing system first purges remnant ink-A from a mixing chamber and out of an extruder tip before ink-B is introduced and can actually be expelled from an extruder tip.

In some embodiments, a 3D printing system includes a combination of an aspirator and a dual-ink or multi-ink blending tip. In some embodiments, an aspirator is a third line connected to a mixing chamber and a microstepped vacuum. In some embodiments, a third line removes ink-A from a mixing chamber before ink-B is loaded into a mixing chamber. In some embodiments, removing ink-A with an aspirator line prior to introducing ink-B reduces printing time. In some embodiments, removing ink from a mixing chamber decreases lag time when modulating blends. In some embodiments, an aspirator line can operate in discrete, intermittent periods of suction, or can be run continuously during printing.

Charged Extruder

In some embodiments, printing as described herein involves generation of a Taylor cone structure at the ink nozzle to achieve higher resolution structures. In some embodiments, an electrical gradient stretches a solution droplet of bio-ink composition during the print into a Taylor cone. In some embodiments, a Taylor cone shape is designed to produce a finer resolution and compensate for imperfect printing surfaces.

In some embodiments, a 3D printer for use in the practice of the present invention and for forming a deposition having a Taylor cone extrusion profile includes: a print head having a conductive extruder nozzle configured to provide bio-ink composition onto a surface (a printing surface) of a substrate; a ground electrode; and a power supply configured to apply a voltage between an extruder nozzle and a ground electrode. In some embodiments, a 3D printer of the present invention may further include a controller configured to cause a bio-ink composition to form a Taylor cone as it exits an extruder nozzle. In some embodiments, a 3D printer of the present invention may further include a controller configured to control an applied voltage to selectably contact and disengage a Taylor cone from a surface in a predetermined manner in accordance with a programmed pattern.

In some embodiments, a 3D printer, as illustrated in FIG. 10 100 for printing bio-ink compositions uses two or more actuatable (e.g., microstepped) extruders to modulate ink blend during printing to produce gradients or anisotropic properties. In some embodiments, multiple microstepped extruders 110a, 110b, and 110c (collectively referred to herein as extruders 110), as shown in FIG. 10, can be used to independently modulate these blends to spatially and temporally control solubility and mechanical properties of prints.

Referring to FIG. 11, standard liquid behavior at the tip of the extruder 110 results in a rounded, generally spherical liquid profile, which may lead to gaps in an intended print line where dips occur, as illustrated.

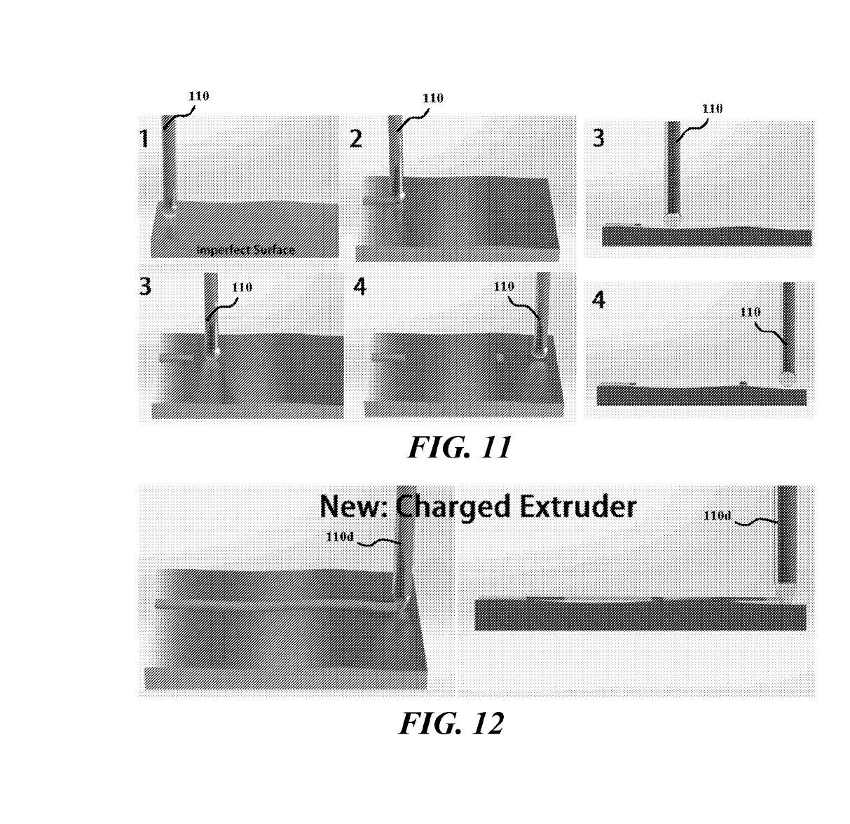

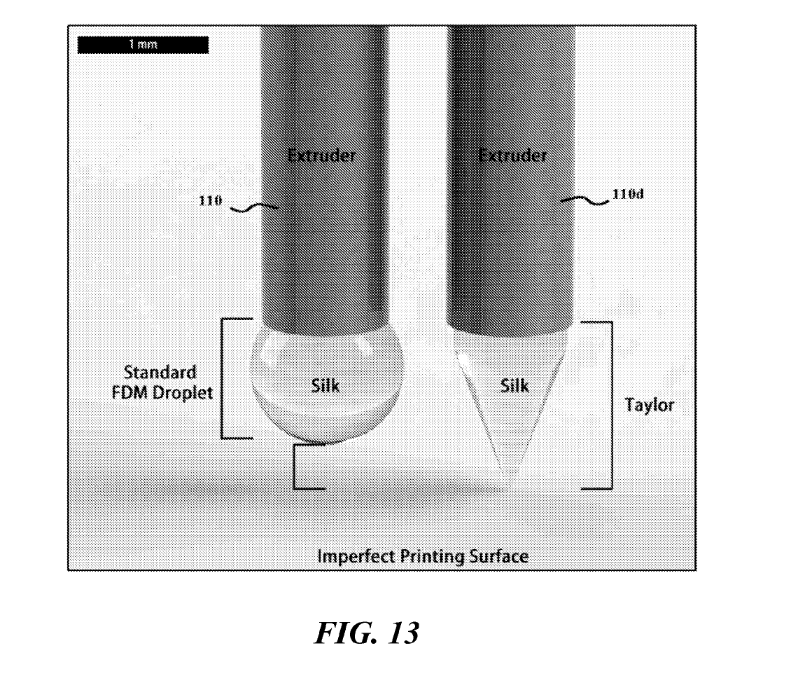

An improved extruder 110d is illustrated in FIG. 12. This arrangement utilizes an electrical gradient between the extruder 110d and an electrode positioned below the substrate to stretch the solution “droplet” into a Taylor cone producing finer resolution as compared to the standard extruder that compensates for imperfect printing surfaces. FIG. 13 shows the non-charged extruder 110 with a standard droplet profile and the electrically charged extruder 110d with a droplet having the profile of a Taylor cone.

In some embodiments, a bio-ink composition droplet shaped as a Taylor cone remains in solution until crystallization occurs at a printable surface. In some embodiments, an applied voltage does not alter or affect crystalline properties of a bio-ink composition solution as it flows from a charged extruder. In some embodiments, bio-ink compositions having a Taylor cone shape substantially concurrently self-cure upon printing, extruding, and/or depositing on a printable surface. In some embodiments, bio-ink compositions having a Taylor cone shape have a short drying and/or curing time after printing, extruding, and/or depositing on a printable surface. In some embodiments, a short drying and/or curing time is in a range between about 0.1 seconds and about 600 seconds. In some embodiments, drying and/or curing time is dependent on a layer thickness. In some embodiments, drying and/or curing time is dependent on a volume of ink. In some embodiments, drying and/or curing time is dependent on environmental factors. In some embodiments, environmental factors include, for example, temperature and/or humidity.

In some embodiments, methods of the present invention include applying a voltage to a bio-ink compositions while flowing from a print head. In some embodiments, applying a voltage in such a manner will cause disclosed bio-ink compositions to form a Taylor cone. In some embodiments, methods further comprise contacting a tip of a Taylor cone with a substrate. In some embodiments, methods include: applying a voltage while dragging a Taylor cone across a surface of a substrate, thereby printing an ink on a surface of a substrate along a path defined by movement. In some certain embodiments, methods of the present invention further include electrically controlling an applied voltage to selectably contact and disengage a Taylor cone from the surface. In some embodiments, an applied voltage, for example, is at least about 0.25 kV, is at least about 0.5 kV, at least about 1 kV, at least about 1.5 kV, at least about 2 kV, at least about 2.5 kV, at least about 3 kV, at least about 3.5 kV, at least about 4 kV, at least about 4.5 kV, at least about 5 kV, or combinations thereof wherein the voltage is fluctuated between and among any of these.

In some embodiments, provided 3D-printing methods include steps of applying a voltage between a conductive extruder nozzle of a print head through which a bio-ink composition is printed and a ground electrode on a side of a substrate onto which the bio-ink composition is printed, which side is opposite the print head.

In some embodiments, provided 3D-printing methodologies include steps of rotating a substrate onto which a 3D structure is being printed relative to a print head through which a bio-ink composition is printed via formation of a Taylor cone, while dragging a Taylor cone across a rotating substrate surface so that a tubular structure is formed. In some such embodiments, a substrate may be rotated about an axis that is perpendicular to a direction of bio-ink composition flow from a print head.

Due to common transition toward more sophisticated design, in vitro techniques using molded or cast scaffolds may now be considered “last-generation” or obsolete for many applications. However, prior work employing these techniques has emphasized the necessity of complex composite structures to proper engineering of tissues. Because there still remains of a lack of understanding of what makes the ideal scaffold, rapid prototyping (“RP”) is uniquely suited to scaffold fabrication due to its capability to generate a rapid series of programmable variables for study. The G programming language is the most widely used numerical control programming language for creating such machine instructions for computer-aided engineering in automation. (See Jeon, K.-H. et al., Development of an Automated Freeform Construction System and its Construction Materials, In Proceedings of the 30th International Symposium on Automation and Robotics in Construction and Mining; International Association for Automation and Robotics in Construction: Montreal, Canada, 2013; pp. 1359-1365 and Sinha, S. K., Automating Facing Operation on a CNC Machining Centre, 2 International J. Engineering Science and Technology, 7616-7618 (2010)). Stepper motor controlled robotics facilitate reproducibility and throughput with sub-micron level control of mechanical components. (See Jordan, S. C. et al., Design Considerations for Micro- and Nanopositioning: Leveraging the Latest for Biophysical Applications, 10 Current Pharmaceutical Biotechnology, 515-521 (2009) and Otsuka, J. Nanometer Level Positioning Using Three Kinds of Lead Screws, 3 Nanotechnology (1992). Simply put, RP enables fabrication of complex geometrical structures that cannot be accomplished with any other method.

Over the past decade, RP technologies originally developed for non-biomedical applications have demonstrated potential for bioprinting and biofabrication. (See Hutmacher, D. W. et al., Scaffold-Based Tissue Engineering: Rationale for Computer-Aided Design and Solid Free-Form Fabrication Systems, 22 Trends in Biotechnology, 354-362 (2004); Peltola, S. et al., Review of Rapid Prototyping Techniques for Tissue Engineering Purposes, 40 Annals of Internal Medicine, 268-280 (2008); Gross, B. C. et al., Evaluation of 3D Printing and its Potential Impact on Biotechnology and the Chemical Sciences, 86 Analytical Chemistry 7, 3240-3253 (2014); Tasoglu, S. et al., Bioprinting for Stem Cell Research, 31 Trends in Biotechnology, 10-19 (2013). Modern computer numerically controlled RP is capable of producing small to large physiologically relevant structures for the aim of replicating biomedical implants or organ geometry. Programmable microcontrollers and high-resolution stepper motors enable RP to generate precisely modulated variables such as geometry, porosity, mechanics, or biological components, with high reproducibility. The most appropriate RP methods for tissue engineering are those which are considered additive techniques, in that fabrication of 3D objects progresses from the bottom-up as a series of cross sections, and does not require milling or molding. A summary of additive techniques is provided in Table 3. These additive techniques are capable of generating 3D structures in physiologically relevant sizes with interlocking components or hollow structures, such as organs. Software converts an original digital design into a series of digital cross-sections. A digital cross-section for each layer is subsequently converted into a guide for each successive print layer. Each technique supports a particular range of control over matrix architecture, mechanical properties, degradation, and biological components. Exploitation of these features used in conjunction with application specific ink formulations creates a platform for fabricating patient customized medical treatments.

TABLE 3
3D rapid prototyping strategies adopted for experimental biomedical applications

| Fabrication Technique | Ink Material | Cure Method | Technique Attributes |
|---|---|---|---|
| Stereolithography | photopolymers | laser polymerization | Laser allows fine features but exhibits shrinking |
| (SLA) | | ultraviolet | and loss of resolution after cure |
| | | polymerization | Weak mechanical properties necessitate UV oven |
| | | oven cure | cure post-processing |
| | | | Damage from UV oven and laser prevent |
| | | | encapsulation of cells and bioactives during the |
| | | | fabrication process |
| Selective Laser Sintering | UHMW polymers | sintering laser brings | CO₂ laser beam provides fast & consistent sintering |
| (SLS) | ceramic powders | powder to glass | of powdered polymers |
| | | transition | Mechanical properties are rigid and suitable for |
| | | | ceramic bone scaffolds |
| | | | Not feasible to obtain porosity with calcium |
| | | | phosphates |
| | | | Damage from laser prevents encapsulation of cells |
| | | | and bioactives during the fabrication process |
| 3D Powder Printing | binder solution for | binder solution is | Print head deposits binder solution onto a layer of |
| (3DP) | powder materials | printed onto a powder | powdered polymer |
| | | bed | Printing can be performed under ambient |
| | | binder solution may | conditions |
| | | contain crosslinking | Water can be used as binder solution to facilitate |
| | | agents | incorporation of bioactives, however prints will be |
| | | | water-soluble, necessitating lengthy post- |
| | | | processing which may be deleterious |
| | | | Unbound powder is difficult to remove from |
| | | | cavities which inhibits porosity |
| Fused Deposition Modelling | thermoplastics | cooling after extrusion | Thermoset printing technology enables a broad |
| (FDM) | | through heated print | range of mechanical properties |
| | | head liquefier | Cells cannot be encapsulated during the fabrication |
| | | | process due to high processing temperature. |
| Direct-Write Assembly | polymer solutions | polymer solution | Printing is compatible with a variety of biomaterials |
| (DWA) | photopolymers | extruded into bath of | Biological components must be added in a separate |
| | thermoplastics | polymerizing agent | step due to the toxicity of the polymerizing solution |
| | | | bath. |
| Robotic Dispensing | polymer solutions | polymerizing agent | Printing is compatible with a variety of biomaterials |
| (RPBOD) | photopolymers | dispensed locally from | Polymerizing solution dispenser can be |
| | thermoplastics | independent nozzle | programmed independently to produce partial or |
| | | | patterned polymerization |
| | | | Biological components must be added in a separate |
| | | | step due polymerizing solution toxicity. |
| MultiJet 3D Printing | photopolymers | ultraviolet | Photopolymers can be extruded in a continuous |
| (MJP) | | polymerization | bead or deposited as discrete droplets through a |
| | | | series of inkjet heads which are mounted in-line |
| | | | with a UV curing lamp. |
| | | | Damage from UV light may prevent encapsulation |
| | | | of biological components during the fabrication |
| | | | process and require addition in a separate step. |
| Dual-cure 3D Silk Bio-ink | silk fibroin | self-curing | 2-part blend induces insoluble stabilization |
| (D3D) | glycerol | | Also facilitates crystallization via chemical initiation |
| | gelatin | | Allows non-toxic encapsulation of cells and |
| | | | bioactives during the fabrication process |

Laser mediated fabrication techniques such as stereolithography (SLA) and selective laser sintering have been used to accurate replicate 3D geometry for tissue engineering. Utilization of an ultra-violet laser, in SLA, to photopolymerize layers of a liquid polymer can generate soft structures, however additional curing in a UV oven is often required to improve mechanical strength. In contrast, selective laser sintering (SLS) can be used to generate constructs immediately capable of mechanically supporting skeletal implant applications. (See Hutmacher, D. W. et al., Scaffold-Based Tissue Engineering: Rationale for Computer-Aided Design and Solid Free-Form Fabrication Systems, 22 Trends in Biotechnology, 354-362 (2004)). In SLS the laser is used to raise the temperature of biomaterial powders beyond the glass transition generating rigid constructs. Resolution is dependent on laser spot size (80-250 μm) and the occurrence of beam absorption or scattering or heat spreading. The major drawback of these techniques is induced UV and heat laser damage which prevent encapsulation of cells and bioactive molecules during the fabrication process. Two-photon polymerization is an improvement.

Laser-induced damage can be avoided using 3D powder (3DP) printing technology. Rather than laser polymerization, 3DP utilizes a print head to deposit a binder solution, such as water or phosphoric acid, onto a bed of powder powdered biomaterial, such as starch, dextran, gelatin or calcium phosphates. (See Castilho, M. et al., Direct 3D Powder Printing of Biphasic Calcium Phosphate Scaffolds for Substitution of Complex Bone Defects, 6 Biofabrication, 015006 (2014). This technique provides more options for tissue engineering and drug-delivery applications because incorporated bioactive components must not be subjected to the deleterious effects of laser mediated fusion or toxic solvents. However, aqueous binding agents often leave printed objects water-soluble, and require further post-processing. A major limitation of powder systems is the difficulty in removing internal unbound powder from desired negative space such as hollow chambers.

Extrusion-based systems are the most widely used 3D printing approach, and are suited for producing hollow structures. Although sacrificial material may be needed to support a range of hollow geometry, these systems have the potential to deposit biomaterial directly into desired geometry thereby facilitating the incorporation of negative space. Precise positioning control is possible for more than two axes (3D printing) and multiple print-heads (parallelization, blending). Fused deposition modelling (FDM) employs solvent-free thermoplastic materials which are heated to a semi-molten state before extrusion then allowed to solidify on the printing stage. The majority of FDM materials are non-bioresorbable and recreations of 3D structures have been limited to plastics or other materials. Several biomaterials such as PCL and PLLA have demonstrated adequate thermoplastic performance in these systems. These prints exhibit good structural strength but suffer print inconsistencies due to feed-rate surging; an issue related to melt temperature fluctuations.

MultiJet 3D printing (MJP) avoids the need for thermoset polymers by employing a photopolymerizing strategy. Photopolymers can be extruded in a continuous bead or deposited as discrete droplets through a series of inkjet heads which are mounted in-line with a UV curing lamp. MJP enables a broad range of geometry and mechanical properties but cannot reproduce 3D structures for sensitive bio-ink compositions.

Direct-write assembly (DWA) systems enable high resolution 3D prototyping by extruding fluid polymer into a bath of corresponding polymerizing solution or cooling bath. (See Ang, T. et al., Fabrication of 3D Chitosan—Hydroxyapatite Scaffolds using a Robotic Dispensing System, 20 Materials Science and Engineering C, 35-42 (2002); Ghosh, S. et al., Direct-Write Assembly of Microperiodic Silk Fibroin Scaffolds for Tissue Engineering Applications, 18 Advanced Functional Materials, 1883-1889 (2008); Lewis, J. A., Novel Inks for Direct-Write Assembly of 3-D Periodic Structures, 3 Mater. Matters, 4-7 (2008)). Extrusions are polymerized or solidified, into the desired programmed geometries, as they are dispensed into the bath. (See Ang, T. et al., Fabrication of 3D Chitosan—Hydroxyapatite Scaffolds using a Robotic Dispensing System, 20 Materials Science and Engineering C, 35-42 (2002); Ghosh, S. et al., Direct-Write Assembly of Microperiodic Silk Fibroin Scaffolds for Tissue Engineering Applications, 18 Advanced Functional Materials, 1883-1889 (2008). DWA is compatible with a variety of biomaterials however, as with FDM and MJP, cells and other biological components must be added in a separate step due to the toxicity of the polymerizing solution, UV exposure, or high processing temperatures. In general, robotic dispensing (RPBOD) systems are compatible with nearly any material. (See Ang, T. et al., Fabrication of 3D Chitosan—Hydroxyapatite Scaffolds using a Robotic Dispensing System, 20 Materials Science and Engineering C, 35-42 (2002); Ghosh, S. et al., Direct-Write Assembly of Microperiodic Silk Fibroin Scaffolds for Tissue Engineering Applications, 18 Advanced Functional Materials, 1883-1889 (2008). The robotic dispensing approach does not require a polymerizing bath. If needed, dispensing of polymerizing agents from a separate nozzle can be programmed independently to produce partial or patterned polymerization.

For biomedical applications, RP hardware limitations, limitations on printer technologies, and limitation of capabilities of printer robotics exist, however, these limitations are often overshadowed by limitations on bio-ink compositions themselves and challenges associated with the RP material and curing strategy. Indeed, modern robotics are often more than technically capable of printing high resolution structures to accurately recreate the solid, hollow, and chambered geometries of many organs and tissues such as bone, vasculature, and the heart. Multi-motor stepper controlled robotics facilitate reproducibility and throughput with a sub-nanometer level of control of print-head positioning and extrusion and such precise positioning control is possible for more than two axes (i.e. capable of printing in three dimensions).

Alternative strategies for incorporating biopolymers as printable inks have been attempted. Solvent-based solutions exhibit evaporation dynamics with significant time-dependent volume changes which present interlayer challenges. The majority of RP utilizes thermoplastics. Replicas fabricated using these materials can be used to generate implant structural components or simulate tissues mechanical properties. Though capable of mimicking extracellular environments, these materials cannot reproduce the extracellular environment needed to regenerate tissues and their use comes with the same pitfalls as with plastics as above described. Many of the biopolymers require chemical crosslinking to preserve a reasonably useful structure or need UV curing. Curing is applied differently depending on the RP technique, but regardless of the technique, UV curing and/or crosslinking can damage structures and inhibit incorporation drugs and/or cells and ultimately limit applicability to physiological environments. To bypass these limitations, strategies which forgo structural integrity to produce patterned depositions of cellular matrix gels have also been practiced. Gel properties facilitate three-dimensional geometry and provide patterned adhesion between printed elements, but the finished prints do not have appreciable structural integrity. Without such integrity, these print strategies are constrained to application with in vitro models insufficient for bioprinting.

As described above, bio-based printing to date has experienced limited applicability largely due to the inability to formulate bio-ink compositions capable of forming sharply defined boundaries; the inability to print bio-ink compositions that retain their structure and mechanical properties when exposed to printing of subsequent ink layers, exposure to solvents, and/or exposure to physiological environments; and the inability to repeatedly generate a flow of material with uniform velocity and volume such that the flow is capable of retaining contact between an extruder and a surface when ejected from a nozzle.

For more than a decade, material development has been recognized as a rapid scaffold prototyping bottleneck. It is clear, that ultra-violet light, chemical cross-linking, and high temperatures will destroy many biologically active additives.

Moreover, printing strategies which utilize cellularized matrix gels and cell-pastes, but forgo deleterious curing mechanisms, have been developed for tissue engineering applications. However, the majority of these prints lack initial mechanical strength and are vulnerable to a wide range of external conditions resulting in melting, dissolution, or warping of printed structures. (See Marga, F. et al., Toward Engineering Functional Organ Modules by Additive Manufacturing, 4 Biofabrication, 022001 (2012). This loss of structure is what necessitates preservative post-processing treatments. However, these treatments negatively affect the biological activity of living elements and the incorporated factors. This compromise between structural strength and biocompatible processing severely limits potential applications such as biomedical implants and modern composite scaffolds with structural components and microfluidic vasculature.

Various embodiments, according to the present invention are described in detail herein. In particular, the present invention provides, among other things, bio-ink compositions and methods related to manufacturing such compositions.

In some embodiments, bio-ink compositions as described herein are particularly useful in formation of medical devices, surgical devices, tissue engineering, imaging, optoelectronics, photonics, therapeutics, synthetic biology, drug delivery, and/or a variety of consumer products.

In some embodiments, bio-ink compositions further include agents and/or additives. In some embodiments, agents and/or additives incorporated into bio-ink compositions are therapeutic, diagnostic, and/or preventative. In some embodiments, agents and/or additives incorporated into bio-ink compositions are releaseable. In some embodiments, agents and/or additives incorporated into bio-ink compositions are configured as markers and/or indicators.

In some embodiments, the present invention is directed to methods of using bio-ink compositions to print, extrude, and/or deposit bio-ink compositions to generate 3D structures, and improved apparatus for generating such 3D bio-ink composition structures.

Among other things, the present disclosure provides bio-ink compositions that are suitable for forming structures at physiologically relevant sizes and with high resolution. In some embodiments, bio-ink compositions are suitable for forming such high resolution structures in three dimensions. In some embodiments, bio-ink compositions suitable for forming high resolution 3D structures as described herein are configured to be printed, extruded, and/or deposited in layers. In some embodiments, bio-ink compositions are printed, extruded, and/or deposited in multiple layers. In some embodiments, bio-ink compositions are printed, extruded, and/or deposited in individual layers that are successively stacked atop one another without damaging the structural integrity or resolution of printed material.

In some embodiments, bio-ink compositions suitable for forming high resolution 3D structures are configured so that when printed, extruded, and/or deposited crystallized layers form. In some embodiments, crystallized layers self-cure and/or form without a need for a distinct curing step. In some embodiments, crystallized layers immediately form when inks are printed. In some embodiments, crystallized layers formed from bio-ink compositions are substantially insoluble, so that when exposed during printing of subsequent layers, solvents, and/or physiological conditions, printed structure maintain their resolution and integrity. In some embodiments, printed substantially insoluble crystallized layers do not decompose, degrade, denature, and or delaminate when exposed to printing of subsequent layers, solvents, and/or physiological conditions.

In some embodiments, curing of printed articles includes selecting a drying time by varying a layer thickness, temperature, humidity, or combinations thereof during deposition. In some embodiments, a 3D printing slicing algorithm accounts for this delay time when generating motor programs.

In some embodiments, provided bio-ink compositions are biocompatible. In some embodiments, provided bio-ink compositions are biodegradable. In some embodiments, provided bio-ink compositions are biocompatible and biodegradable.

In some embodiments, bio-ink compositions form partially soluble crystallized layers that are characterized in that partially soluble crystallized layers dissolve, degrade, denature, and/or decompose over a predetermined time and/or a shortened time relative to a substantially insoluble crystallized layer.

In some embodiments, bio-ink compositions further include agents and/or additives. In some embodiments, agents and/or additives are particularly useful, for example as therapeutics, preventives, or diagnostics. In some embodiments, agents or additives are incorporated into inks as described herein. In some embodiments, bio-ink compositions including such agents or additives are printed, extruded, and/or deposited in multiple layers without damaging and/or killing such agents or additives and while maintaining structural integrity and resolution. In some embodiments, agents and/or additives incorporated into bio-ink compositions are releaseable. In some embodiments, agents and/or additives incorporated into bio-ink compositions are configured as indicators or markers.

In some embodiments, bio-ink compositions as described herein are suitable for forming high resolution 3D structures as described herein. In some embodiments, bio-ink compositions can be configured to form specialized tissue scaffolds and patient specific implant geometries. In some embodiments, such specialized tissue scaffolds or specific implant geometries may be designed and configured on command.

In some embodiments, bio-ink compositions as described herein comprise polypeptides and humectants.

In some embodiments, a polypeptide is or comprises silk fibroin and glycerol is a humectant. In some embodiments, glycerol is incorporated as an additive specifically for the purpose of printing inks into insoluble crystallized layers upon which additional layers can be subsequently printed. Otherwise, subsequent print layers of fresh “ink” which may contain solvent, would dissolve the previous print layer, as they are printed.

As indicated above and unlike ubiquitous aqueous silk solutions used in tissue engineering applications, polypeptide:humectant bio-ink compositions, for example, silk:glycerol ink solutions dry to an insoluble crystallized state during printing. There is no need for intermittent chemical treatments, lengthy evaporation and/or annealing periods, or electrogelation, so that there is no need for any additional alcohol treatments, shearing, gelling, e-gelling, or crystallization steps between printing of subsequent layers. Humectants, such as glycerol confers this ability.

Prints produced using a blend of glycerol and silk exhibit more consistent geometry and more regular features. Sharp angles and clean edges are more easily achieved and maintained once transferred to simulated physiological environments, or completely submersed in water/PBS. These prints are robust and generate impressive mechanical strength comparable to traditional regenerated silk fibroin films.

Robust bio-ink compositions as disclosed herein in fact permit techniques, such as fused filament fabrication (i.e. 3D printing) without showing side-effects from heat damage. In some embodiments, new ink layers are easily printed on top of a dried crystallized layer, thereby creating 3D polypeptide based structures. Thus, non-thermoplastic bio-ink compositions may be used to generate fused laminar structures, which are comparable to thermoset 3D printing, but biocompatible. Bio-ink compositions for use in accordance with the present invention permit multi-layer fused filament fabrication without requiring steps which would damage sensitive molecules incorporated as “additives” such as drugs, growth factors, or even cells.

### EXEMPLIFICATION

### Example 1

The present example describes preparation of certain bio-ink compositions in accordance with the present invention.

Preparation of Aqueous Silk Solution: Silk solutions were prepared using procedures previously established and disclosed in D. N. Rockwood, et. al., 6 Nature protocols 1612 (2011) which is hereby incorporated by reference in its entirety herein. Briefly, 5 grams of B. mori silkworm cocoons were immersed in 1 L of boiling 0.02 M Na₂CO₃ solution (Sigma-Aldrich, St. Louis, Mo.) for 10, 30 or 60 minutes, subsequently referred to as 10 mb, 30 mb and 60 mb respectively, to remove the sericin protein coating. Degummed fibers were collected and rinsed with distilled water three times, then air-dried. The fibers were solubilized in 9.3 M LiBr (20% w/v) (Sigma-Aldrich, St. Louis, Mo.) at 60° C. for 4 hours. A volume of 15 mL of this solution was then dialyzed against 1 L of distilled water (water changes after 1, 3, 6, 24, 36, and 48 hours) with a regenerated cellulose membrane (3,500 MWCO, Thermo Scientific, Rockford, Ill. or 3500 MWCO, Slide-A-Lyzer, Pierce, Rockford, Ill.). The solubilized silk protein solution was then centrifuged twice (9700 RPM, 20 min., 4° C.) to remove insoluble particulates. Protein concentration was determined by drying a known mass of the silk solution under a hood for 12 hours and assessing the mass of the remaining solids.

Preparation of Silk:Humectant Blends Solution: An humectant additive was blended with silk in a ratio of 75:25 (w/w dry) and another normalized for molar mass of the additive.

### Example 2

The present example describes design of a printed article in accordance with the present invention.

Design of a Printed Layer: Printed layer designs were modeled using CAD, for example SolidWorks. Instructions were accomplished via manual programming written in C++ or by generating interpreted programming Interpreted programming was generated in four steps. After desired geometry was designed using CAD, it was converted into layer stacks using a slicing algorithm. Each layer in the stack was converted to G-code positioning commands. The G-code required manual programming edits to be compatible with the custom printing system. After editing and simulating the programmed run with host-controller software, it was possible to interpret the modified code directly for use with the Arduino microcontroller.

### Example 3

The present example describes forming a printed article from a design in accordance with the present invention and evaluation thereof

Formation of a Printed Layer: High resolution fusions of independent elements were printed on a printable surface. Precision shapes were created. Solid structures are fabricated as film laminates. Meshwork structures were fabricated by printing sequential slices of an intermittent print scheme. The perimeter of each printed slice was a reconstruction of the corresponding cross-section from original CAD geometry. Depending on the height of the print geometry, the layer-by-layer additive fabrication of hollow and porous structures required the use of support material. A support material should be soluble, yet support direct contact with structural print extrusions. Remnant solvent in the structural print extrusions should not be sufficient to induce immediate dissolution of a support print.

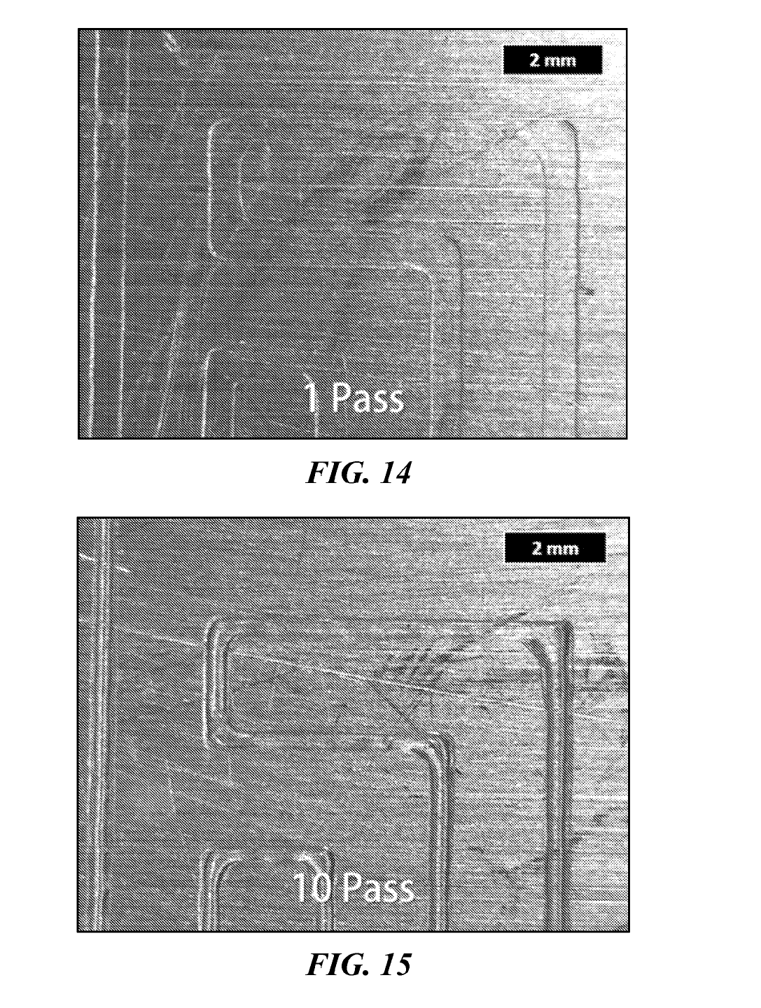

Film- and mesh-based 3D prints were feasible. Silk/glycerol bio-ink composition facilitated predictable micro-deposition volumes and consistent layer height for laminate prints. Tensile properties of laminates are not significantly different from films cast as a single layer (data not shown). FIG. 14 shows a printed layer using a single pass of a printed silk:glycerol layer. FIG. 15 shows a printed 3D-layer using ten passes of a printed silk:glycerol layer.

Evaluation of a Printed Layer: Precision of the printed layers was optimized with nozzle geometry, extruder clearance, rate consistency, and surface tension. Configurations were optimized by depositing isolated and contiguous print elements in the form of lines and microdroplets. Consistency of isolated and contiguous shapes and size limitations were evaluated using parallel lines of different alternating blends. Fusion of borders was assess as was the degree to which unfavorable blending has occurred. Fusion of borders was evaluated by measuring the tensile strength of rectangular prints perpendicular to parallel elements.

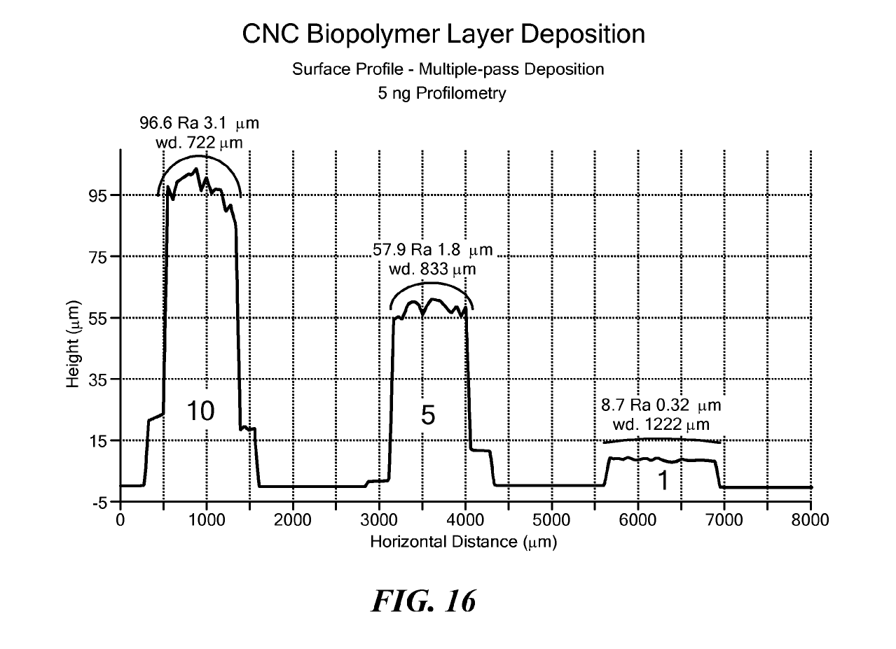

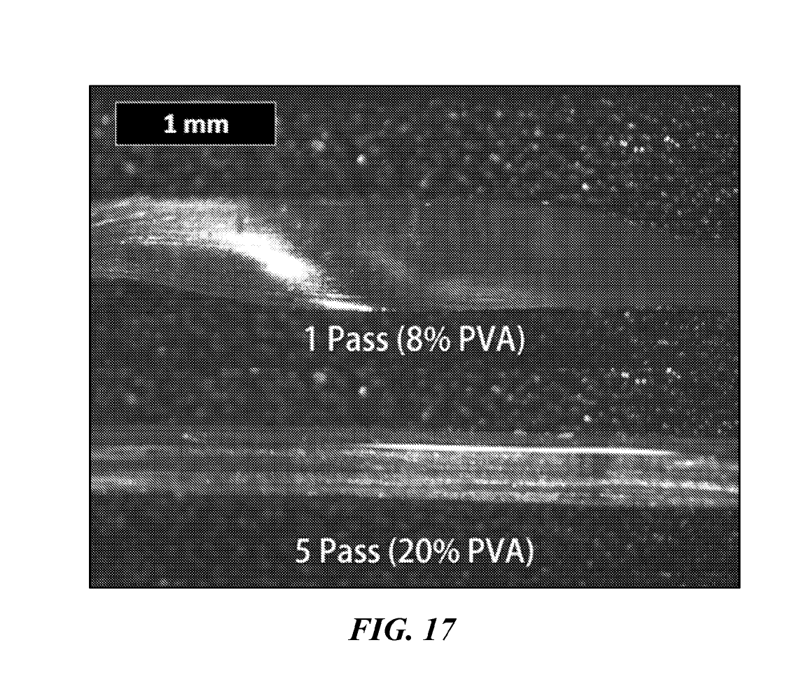

Printed samples were allowed to form into films, then subjected to solubility in PBS @ 20° C. and observed with phase contrast. Crystallinity was compared using FTIR. Tensile properties were measured. Second printed layers of laminated samples were tested for delamination strength. Layers were delaminated to evaluate moduli and UTS of each print as an indication of cohesive strength and interlayer adhesion when compared to the tensile properties of individual layers. Contact angle was measured to evaluate spreading of printed elements was similar in order to ensure consistent printing resolution. Viscosity and surface tension were measured. Print buckling was measured. Volumes of discs of printed layers were measured before and after dissolution and compared using interferometry. To quantify volume reduction due to solvent evaporation dynamics, print lines of various blends and extrusion rates were deposited and buckling of the surface profile was optically tracked over time. The use of blends which exhibit similar buckling is useful for printing subsequent slices of a multi-layer stack. FIG. 16 shows profilometry data for three bio-ink composition depositions. Profilometry data shows a surface profile for increasing deposition height with increasing layers of deposition. The profile on the left corresponds to ten passes (or built up layers) of the print head, whereas the middle profile corresponds to five passes and the right profile corresponds to one pass. FIG. 17 shows printed layers of both one pass using a silk:glycerol blend and for five passes using a silk:glycerol blend. Printed layers formed from both one pass and five passes generate thin film prints. These prints, as shown in FIG. 17 were removed (or peeled) from a printed surface without damaging or breaking printed layers showing that silk:glycerol prints are flexible and robust.

### Example 4

The present example describes applications for various printed articles in accordance with the present invention.

Printable Structures: printable structures using bio-ink compositions are limitless, simply depending on the available 3D printer. The printable structures in some examples are bio-compatible implants, while other examples are provided for other medical or non-medical purposes, e.g., consumer goods.

3D Printed Biopolymer Surgical Implants:

Devices fabricated for highly irregular geometries are possible, for example cheekbone implants. Such surgical devices are manufactured from flexible materials ranging from solid plastics to injectable soft tissue fillers. Cheekbone geometry is highly irregular. Biomedical implants fabricated are evaluated in vitro and in vivo for structural integrity of printed components and fusion of layers in addition to industry standard benchmarks in the areas of implant function, resorbability, and chronic injury or toxicity.

### Example 5

The present example describes incorporating radiopaque markers into bio-ink compositions and forming printed articles for imaging and detection thereof

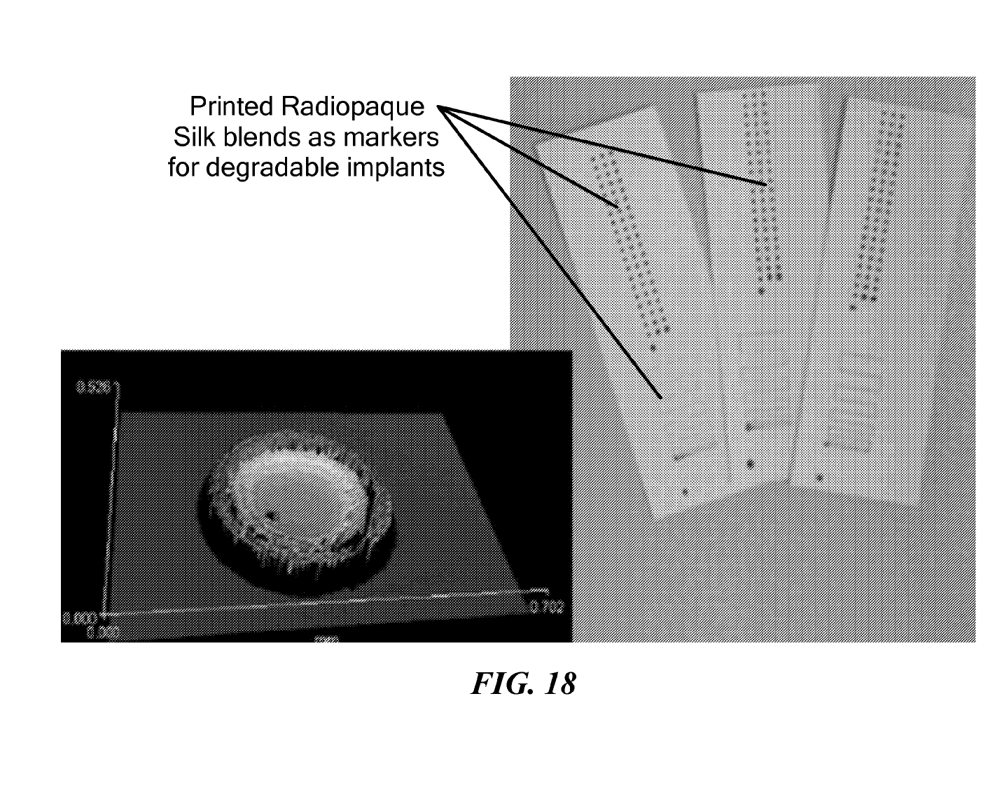

Radiopaque Bio-ink Composition Markers: additives such as iron or magnesium may be incorporated to produce radiopaque inks. Resorbable yet radiopaque protein inks for printing time/event sensitive markers were blended, for polymer implants. The untimely disappearance or persistence of these markers can be tuned to indicate healthy or diseased conditions such as hyperplasia. FIG. 18 shows printed resorbable radiopaque markers for monitoring time dependent events. Resorbable radiopaque markers were printed with about 5% iron w/v, about 10% iron w/v, 15% iron w/v, 20% iron w/v, 25% iron w/v, 30% iron w/v, 35% iron w/v, 40% iron w/v, 45% iron w/v, and 50% iron w/v.

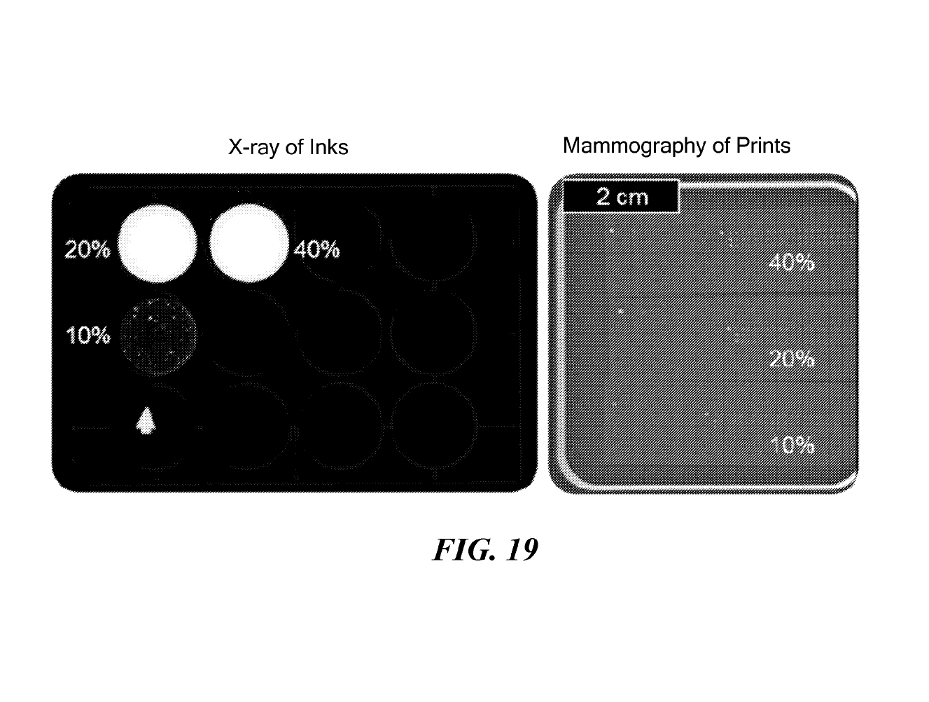

Radiopaque inks were used for implant detection using standard clinical imaging techniques, as shown, for example, FIG. 19 shows X-ray and mammography of radiopaque silk/iron blended inks and subsequent single-layer prints. Percentage values represent w/v ratio of 1 micrometer iron particles to 2% silk. Higher iron content enhanced intensity of detection. Radiopaque ink patterns are discussed in additional detail below.

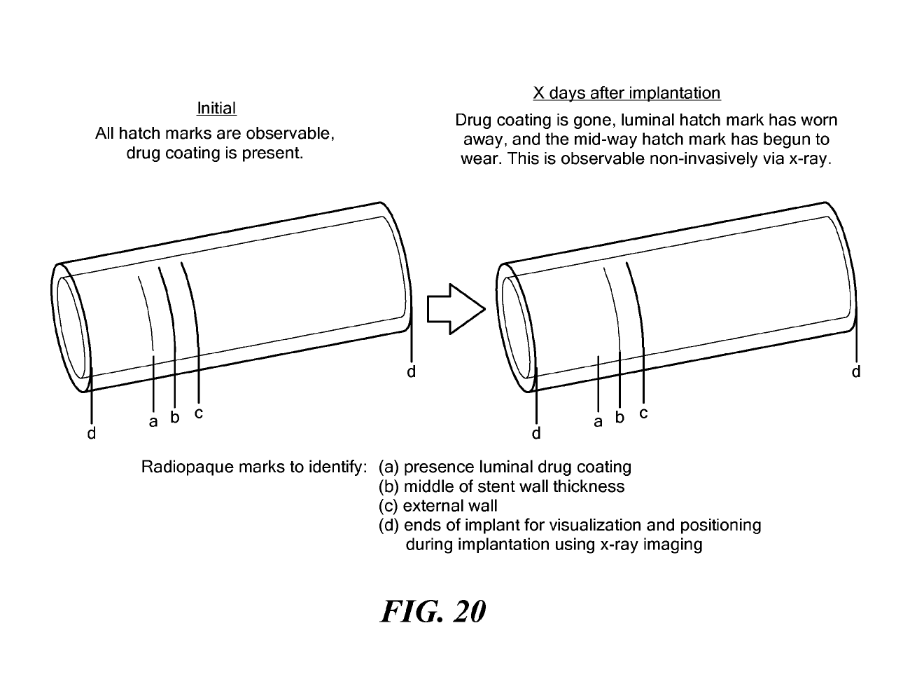

FIG. 20 shows resorbable radiopaque bio-ink composition markers printed onto a polymer implant substrate. In particular, three stripes were applied. As shown on the left image, when all three stripes were observable, the radiopaque ink indicates that a drug coating is present on a polymer implant substrate. Once the radiopaque ink hatch marks wore away, as detectable via X-ray imaging after implantation, it was determined that the drug coating was gone.

In addition to identifying the presence of luminal drug coatings, radiopaque printed bio-ink composition markers identify stent wall thickness and are used to mark the ends of the stent implant structure for visualization of relative locations of walls of a stent or other device and positioning of a stent or other device during an implantation procedure using x-ray imaging.

### Example 6

The present example describes printing, depositing, and/or extruding finely distributed and patterned drug-loaded microdroplets.

Drug-Containing Bio-ink Composition Structural Microdroplets: finely printed and patterned drug-loaded and structural droplets were fabricate in a patchwork composite film. Such films allow more control over drug elution by facilitating programmable schemes which vary by layer.

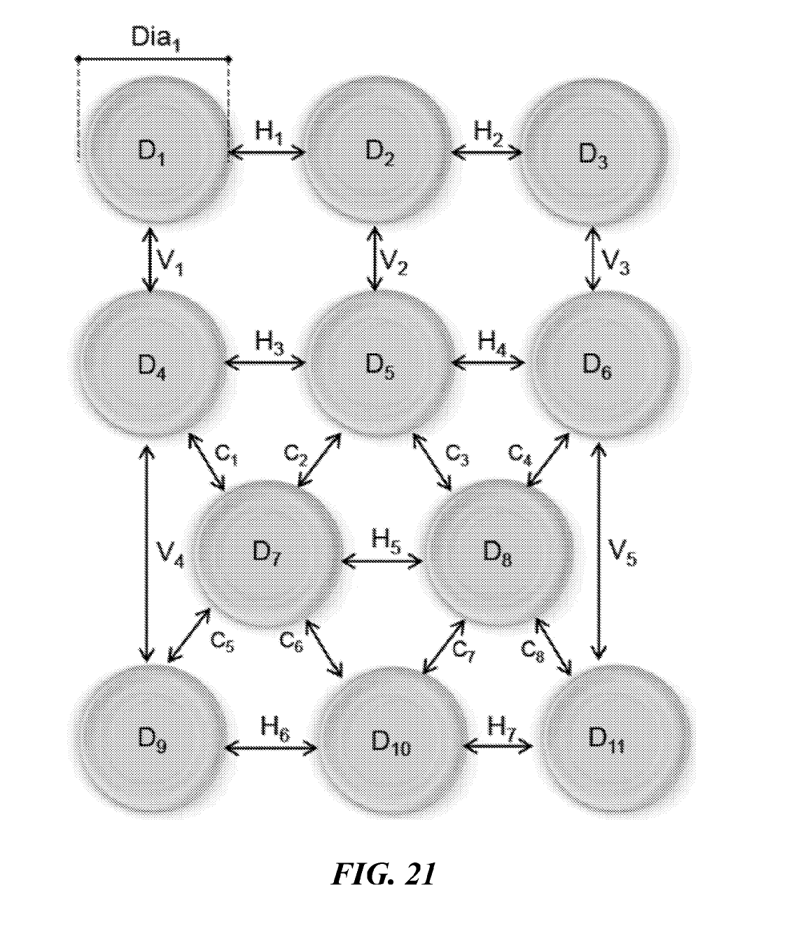

FIG. 21 shows patterned drug-containing bio-ink composition microdroplets for drug elution. The arrangement of relative droplet positions was optimized to influence the degree of flow induced mechanical stress. Flow induced mechanical stress has been shown to affect the droplet degradation rate.

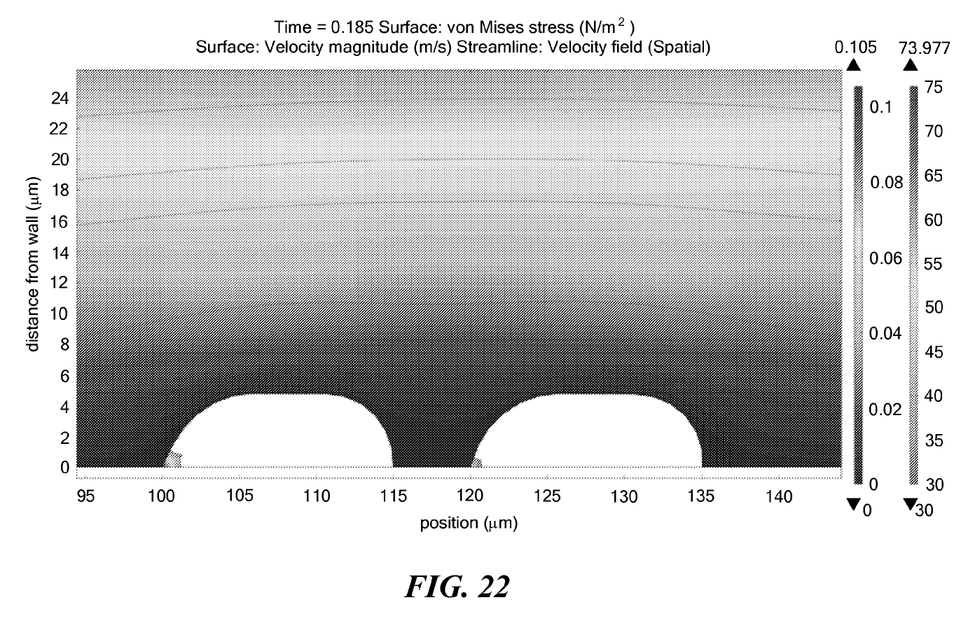

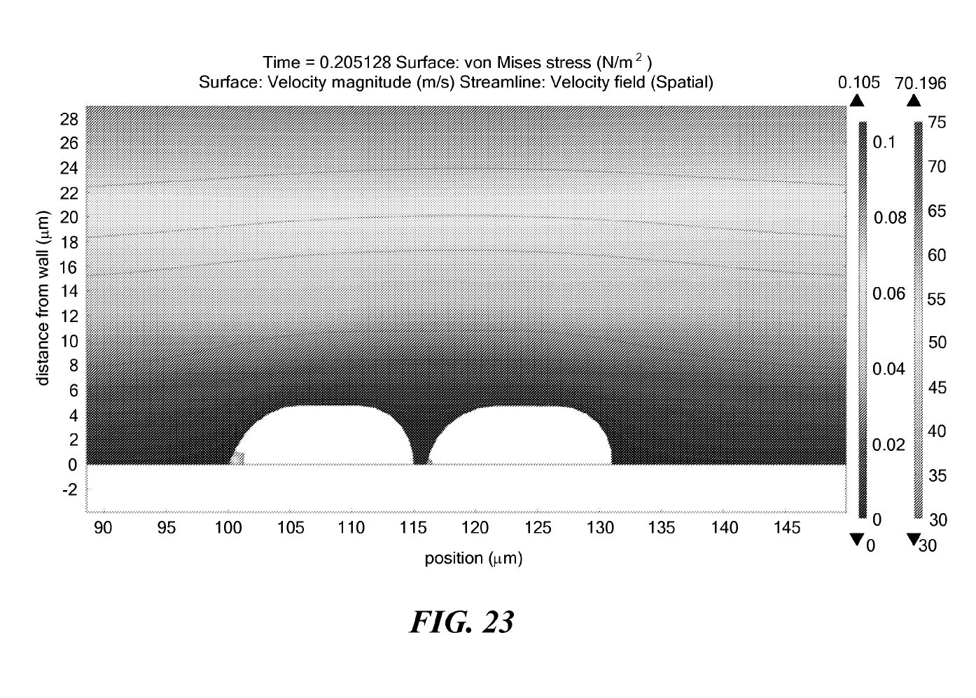

FIG. 22 and FIG. 23 show stress profiles acquired from drug-containing bio-ink composition microdroplet patterns that were exposed to fluid streams.

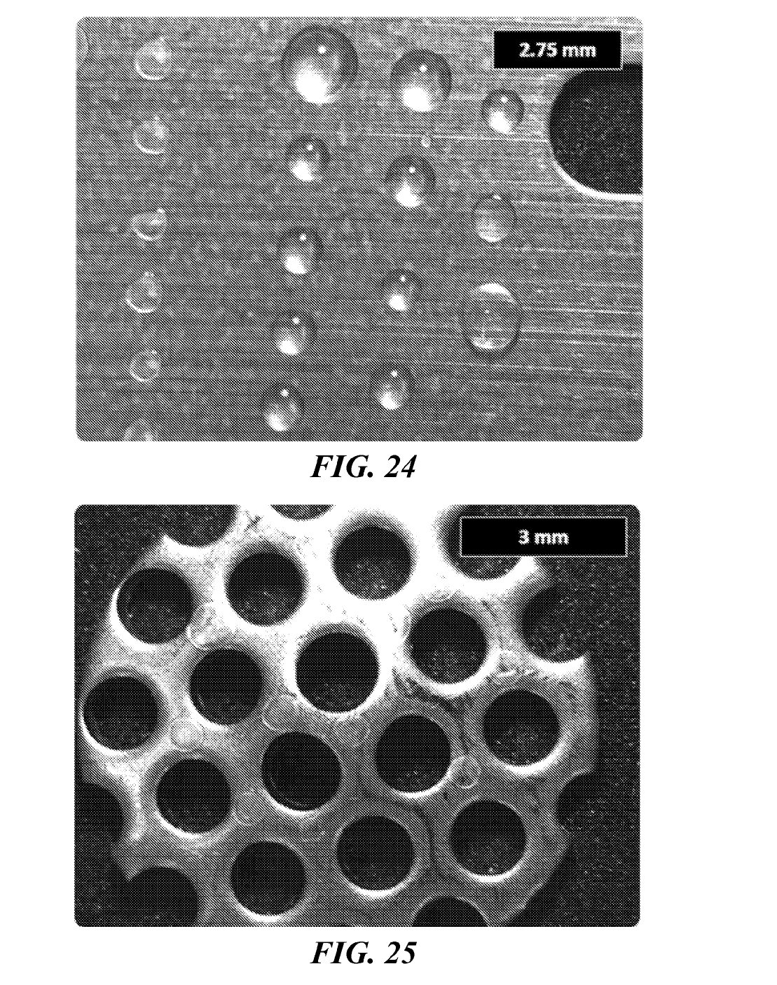

FIG. 24 shows a pattern of drug-containing bio-ink composition microdroplets that were printed on a continuous substrate. FIG. 25 shows a pattern of drug-containing bio-ink composition microdroplets that were printed on a perforated substrate.

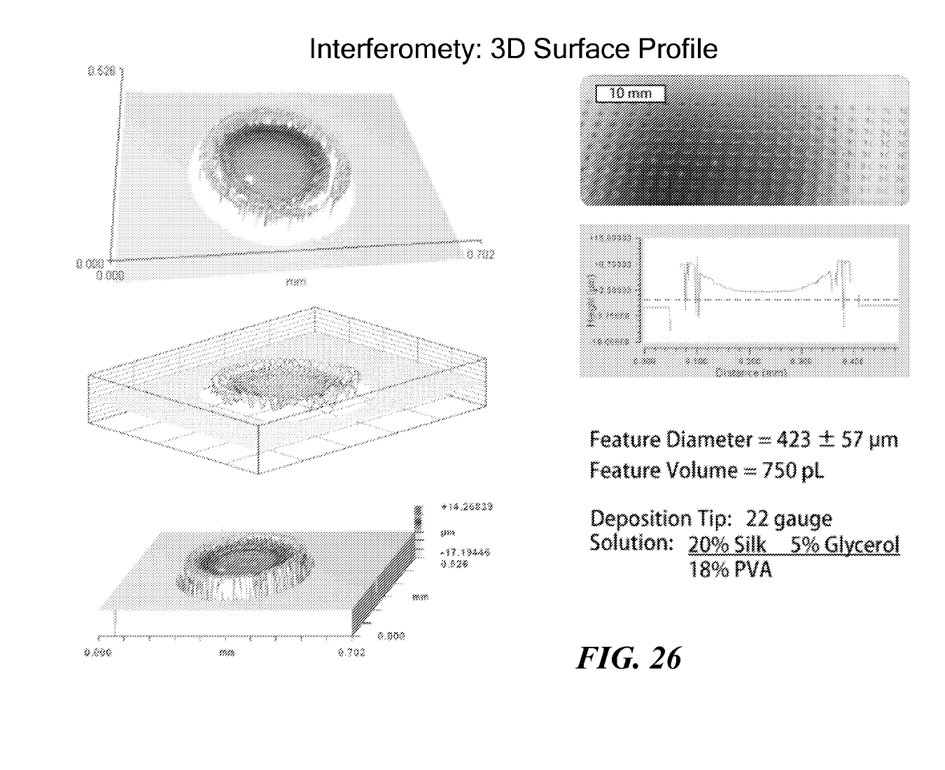

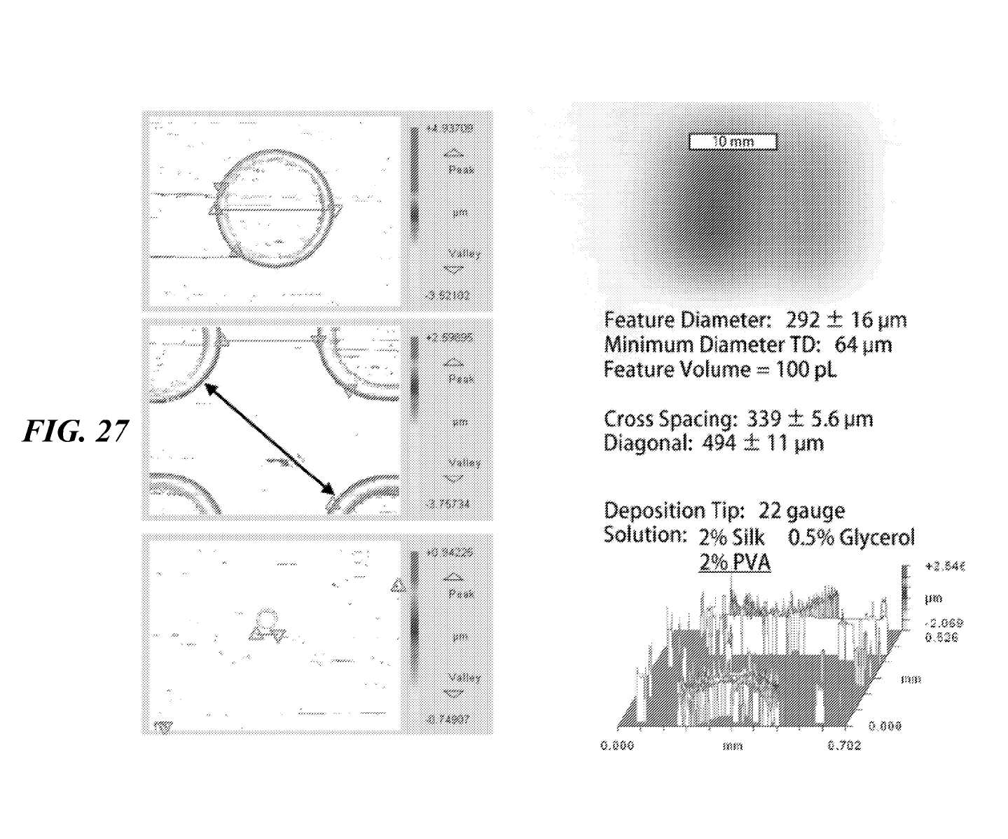

FIG. 26 and FIG. 27 show a an interferometry analysis of a 3D surface profile of a bio-ink composition droplet and pattern of droplets that were printed on a substrate.

### Example 7

The present example describes printing bio-ink compositions on rotatable printing surfaces.

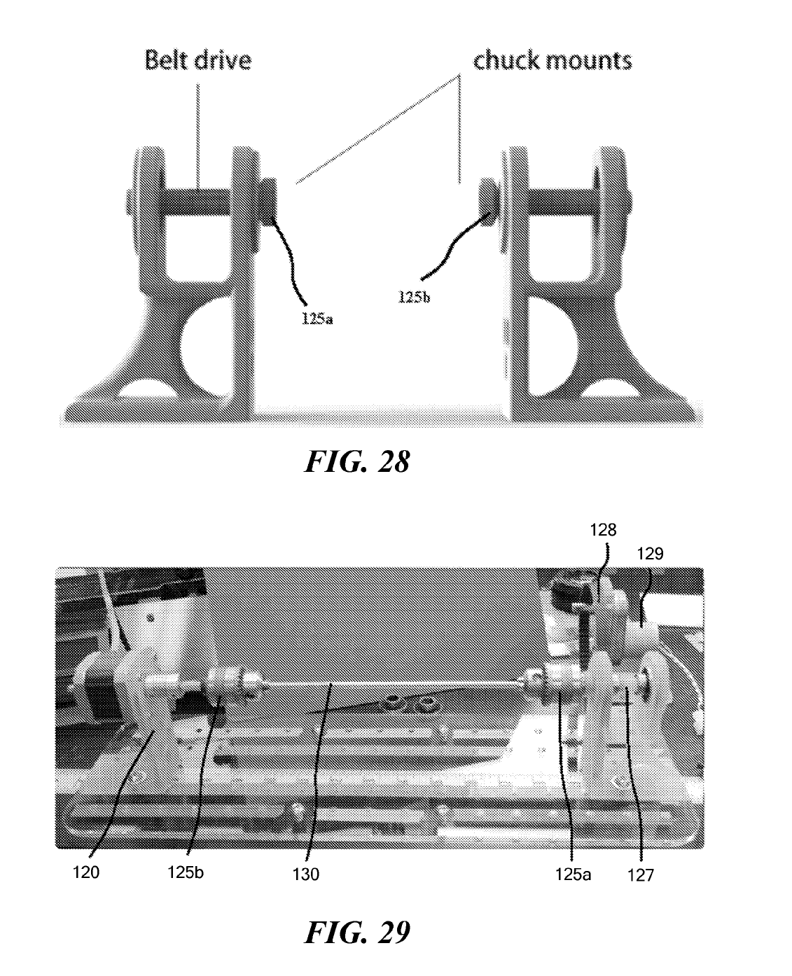

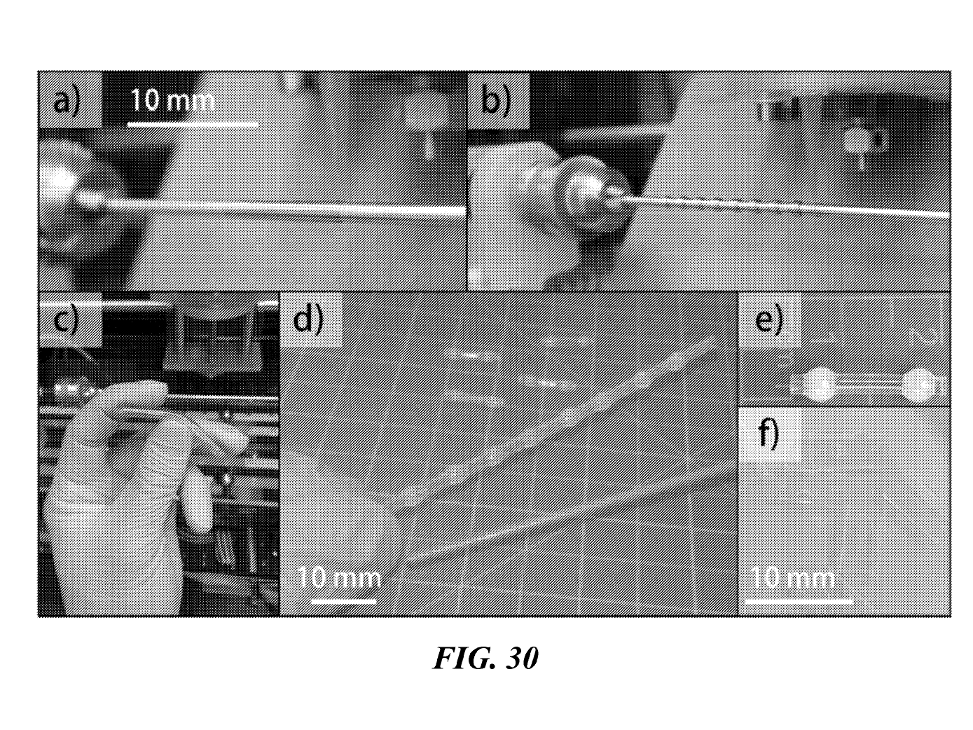

Coating Tubular or Rotatable Surfaces: in addition to near flat printable surfaces, 3D printing system includes the capability to print on rotatable cylinders. Referring to FIG. 28 and FIG. 29, a 3D printer system includes a rotatable substrate mounting system 120. In this manner, the printer 100 is configured to print tubular structures. Such structures may be highly advantageous for surgical implants, allowing, for example, the 3D printing of the example anastomosis device described herein.

The rotatable substrate mounting system 120 included a first and second chuck mounts 125a and 125b, collectively referred to as chuck mounts 125. The first chuck mount 125a included a belt drive sprocket configured to be driven by a belt 128 actuated by a belt actuator 129, e.g., an electric motor. Although specific actuation mechanisms may be described, it should be understood that such examples are in no way limiting and any suitable actuation mechanism may be provided.

The chuck mounts 125 are configured to receive a substrate 130 such that actuation of the chuck system 120 causes the substrate 130 to rotate. In the illustrated example, the substrate 130 has a cylindrical geometry, for example tubing. It should be understood, however, that any desired substrate geometry may be provided and adapted so that rotation in the chuck mounts 125 were possible. The rotatable mounting of the substrate 130 allowed the substrate 130, herein a stainless tube, to be rotated relative to the print head of the printer 100 such that the printed 3D structure conformed to the outer surface of the substrate 130. The printed structure was subsequently removed from the rotatable substrate mounting system. A silk:glycerol ink coating was applied in multiple layer over multiple passes to the tube and created a print layer on the outer diameter of the tube.

### Example 8

The present example describes forming an anastomosis device using bio-ink compositions and method of forming printed articles as disclosed herein.

A Fully Resorbable Drug-Eluting Sutureless Silk Anastomosis Device: was designed for small to large diameter vessels to decrease complexity and ischemic time in vascular reconstructive surgical procedures, which may lead to less invasive cardiovascular anastomosis. An implant was designed to utilize a barb-and-seat compression fitting composed of one male and two female components. The implant body was constructed to be resorbable and capable of eluting heparin. A custom 3D printing system controlled extrusion to fabricate the implants.

Manual suturing is the current gold standard for generating vascular anastomoses. Reconstitution of blood supply via vessel anastomosis remains a technically challenging and time consuming procedure with a steep learning curve for surgeons. Suturing errors such as uneven spacing, inversion of suture walls, and misalignment of the vessel intima can lead to anastomotic leaks, thrombosis, prolonged hospital stays and death. By decreasing the level of technical dexterity required for anastomosis, pathways to robotic or less highly skilled is possible.

Cocoons of the silkworm Bombyx mori were supplied by Tajima Shoji Co. (Yokohama, Japan). Sodium carbonate, lithium bromide, and Glycerol MW 92.09, were purchased from Sigma-Aldrich Corp. (St. Louis, Mo., US). Porcine femoral vessels from 6-9 month old 250 lb. Yorkshire pigs were ordered from Animal Technologies, Inc. (Tyler, Tex., US). Heparin and Fluorescein conjugated Heparin was purchased from Invitrogen Life Technologies (Grand Island, N.Y., US).

Aqueous silk fibroin solutions were prepared following published procedures. Aqueous fibroin solutions were blended with 99% (w/v) glycerol, as previously described, to produce blends of 80:20 (dry weight) silk:glycerol solution.

Silk fibroin was used as a structural material to generate the anastomosis devices, due to its strength and degradability. The silk material was autoclaved for sterilization without loss of mechanical integrity. Glycerol was used in the material. Glycerol is a simple metabolizable non-toxic sugar alcohol ubiquitous in food and pharmaceutical industries. When blended, glycerol stabilized an intermediate conformation of crystallized silk which produced a more flexible yet stable and strong film.

The tubular component of the coupler was fabricated by coating aqueous silk:glycerol (20% dry wt. glycerol) solution on to the Teflon coated stainless steel rods (0.65 to 6 mm diameter) using a microstep controlled extruder and lathe as above described. The coating was allowed to dry and each coating produced a 40 μm thick tubular film layer and subsequent layers were deposited to achieve the target (150 to 300 μm) thickness. Lower concentrations of silk:glycerol can be used to generate thinner layers. The ellipsoid barb tips were produced in a separate step by dispensing 5 to 50 μl of silk:glycerol solution onto the previously coated rods. The final outer diameter of the barbs were equivalent to approximately 125% of the outer diameter of the coated rods.

A micro-stepped extrusion system deposited layers of silk glycerol around Teflon-coated stainless steel rods to fabricate the tubular film components. FIG. 30 shows the following process flow followed for fabrication of an anastomosis device: FIG. 30, subpart (a) provides a step of coating of rods for clip and coupler components; FIG. 30, subpart (b) provides a step of depositing a spherical barb tip for coupler components; FIG. 30, subpart (c) provides a step of removing tubes from rods for clip components; FIG. 30, subpart (d) provides a step of removing tubes with spherical barbs from rods for couplers; FIG. 30, subpart (e) provides a step of trimming coupler components tubes with spherical barbs from rods for couplers; FIG. 30, subpart (f) provides a step of trimming clip components from tubes and creating seats using biopsy punch.

The fully resorbable drug-eluting sutureless silk anastomosis device used three resorbable components; two identical tubular clip sheaths with two opposing holes and the third component is a tubular coupler terminating with ellipsoid barbs at each end.

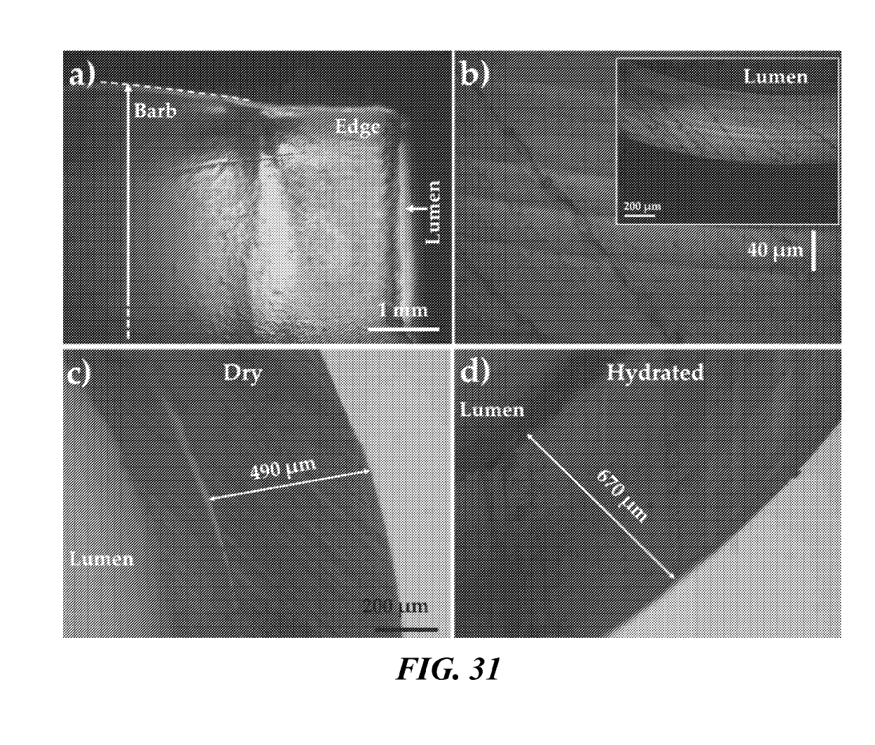

The implant body was produced from silk. The body was stiff in the dry state yet progressively softened when hydrated. Softening eased implantation and allowed the implant to exhibit softer properties after implantation, thereby avoiding stress shielding and minimizing the risk of long-term chronic irritation. Moreover, the hydration of the silk material caused slight swelling slightly after hydration in physiological conditions. FIG. 31 shows that the outer diameter and sidewall thickness of the coupler increased approximately 12% and 30%, respectively, after hydration.

The fully resorbable drug-eluting sutureless silk anastomosis device was fabricated with an implant wall thickness of 300 nm. The maximum radial strength per millimeter of mercury unit pressure drop within the 4 mm vessel model, targeted for in vivo implantation, and produced similar results for smaller caliber vessels.

The fully resorbable drug-eluting sutureless silk anastomosis device was fabricated with a radial strength capable of maintaining radial tension at the coupler bead and clip seat interface. Radial crush resistance of the implant within a latex pressure chamber was dependent on wall thickness. The maximum crush resistance of 4.48 psi was obtained from the couplers of approximately 300 μm wall thickness, which was nearly 45% higher than self-expanding metallic vascular implants. Increases wall thickness marginally increased the crush pressure but also increases flow resistance.

The erosion of the fully resorbable drug-eluting sutureless silk anastomosis device progresses from the luminal surface due to direct contact with fluid flow (e.g. water or blood). Silk degrades into amino acids over the course of weeks to years with no known bioburdens. In contrast to other common degradable polymers (collagens or polyesters such as PLGA), silk has been reported to be less immunogenic and inflammatory and has been reported to be used successfully as implants in small diameter blood vessels in a number of animal studies and as sutures for decades (FDA approved).

Deposition of multiple layers of silk was used to entrap various drugs (antiplatelet, antiproliferative) in each layer of the coupler sidewall which eluted as the couplers erode in vivo. This approach presents a unique opportunity to locally deliver multiple drugs over several time scales to treat a variety of clinical conditions. Ambient processing conditions of silk facilitated incorporation of sensitive drugs.

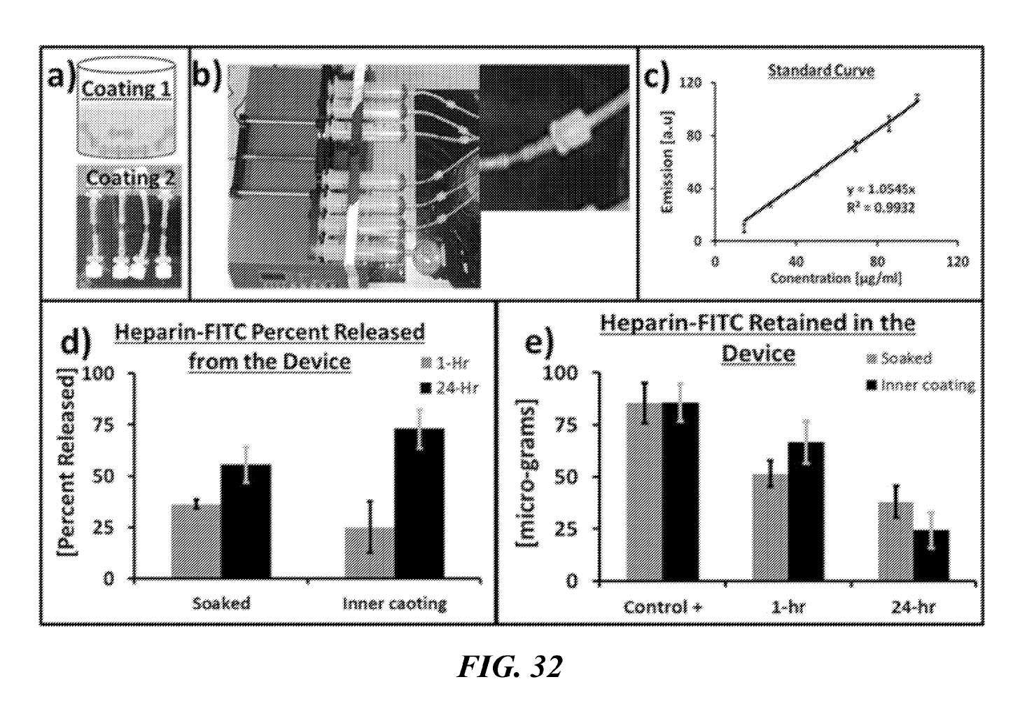

Coupler devices were soaked in fluorescein conjugated heparin solution (0.5 mg/ml in deionized water) using two different techniques. FIG. 32 subpart a shows either that the luminal surface of the couplers were coated with fluorescein conjugated heparin solution or the coupler devices were completely submerged into the solution for 24 hours. FIG. 32 subpart b shows that after equilibration for 24 hours, the couplers were rinsed with deionized water and secured between two segments of silicon tubing mounted in line with a standard perfusion system to mimic dynamic flow conditions for drug release. The devices were perfused at a rate of 2 ml/min for 1 hour and 1 ml/hr for 24 hours using deionized water. The perfused silk couplers were removed from the perfusion system and then dissolved in lithium bromide solution to quantify the remnant drug. The dissolved samples and standards (known amount of fluorescein conjugated heparin in silk/lithium bromide solution) were measured (at 495 nm excitation and 515 nm emission) using a plate reader (Molecular Device, LLC, model: SpectraMax M12, Sunnyvale, Calif.) and plotted in micro-grams. The error bars represent the standard deviation and n=5 per condition and time point.

Luminal surfaces were coated with heparinized-silk or the devices were hydrated with a heparinized solution. Hydrated heparin-loaded devices rapidly released most of the drug. Dry luminally-coated couplers exhibited delayed release. While not wishing to be bound to a theory, it is believed that the delay in release was due to the absorption of the drug during the drying process of the lumen coating. Once the coupler lumen had hydrated during the study the release rate of the remaining drug was similar. By the 24 hour time point, the luminally-coated devices released approximately 20% more heparin than the devices loaded via hydration with heparin solution. FIG. 32 subpart d shows a total quantity of Heprain released from the devices over 24 hours. FIG. 32 subpart e shows the amount of remant drug retained in the devices at 0, 1, or 24 hours.

### OTHER EMBODIMENTS AND EQUIVALENTS

While the present disclosures have been described in conjunction with various embodiments, and examples, it is not intended that they be limited to such embodiments, or examples. On the contrary, the disclosures encompass various alternatives, modifications, and equivalents, as will be appreciated by those of skill in the art. Accordingly, the descriptions, methods and diagrams of should not be read as limited to the described order of elements unless stated to that effect.

Although this disclosure has described and illustrated certain embodiments, it is to be understood that the disclosure is not restricted to those particular embodiments. Rather, the disclosure includes all embodiments, that are functional and/or equivalents of the specific embodiments, and features that have been described and illustrated. Moreover, the features of the particular examples and embodiments, may be used in any combination. The present invention therefore includes variations from the various examples and embodiments, described herein, as will be apparent to one of skill in the art.

What is claimed is:

1. A bio-ink composition, comprising:
a polypeptide, a humectant, and a solvent;
wherein the polypeptide and humectant are present in absolute and relative amounts so that the ink is characterized in that when printed on a substrate, it forms a crystallized layer whereby subsequent additional crystallized layers of the ink can be printed substantially concurrently atop prior layers to form a three-dimensional structure.

2. The bio-ink composition of claim 1, wherein the solvent is substantially free of an organic solvent.

3. The bio-ink composition of claim 1, wherein each crystallized layer is substantially insoluble in water so that the crystallized layers do not dissolve, denature, and/or decompose when exposed to subsequent printed layers.

4. The bio-ink composition of claim 3, wherein the crystallized layer comprises at least 35% β-sheet content.

5. The bio-ink composition of claim 1, wherein each crystallized layer is partially soluble.

6. The bio-ink composition of claim 1, wherein the polypeptide is selected from the group consisting of:
fibroins, actins, collagens, catenins, claudins, coilins, elastins, elaunins, extensins, fibrillins, lamins, laminins, keratins, tublins, viral structural proteins, zein proteins (seed storage protein) and any combinations thereof.

7. The bio-ink composition of claim 6, wherein the polypeptide is or comprises silk fibroin.

8. The bio-ink composition of claim 1, wherein the humectant is or comprises a sugar alcohol, a sugar polyol, or a combination thereof.

9. The bio-ink composition of claim 8, wherein the humectant is selected from the group consisting of:
glycerol; ethylene glycol; 1,3-propanediol; 1,4-butanediol; diethylene glycol; −+ butanetriol; −− butanetriol; erythritol; D,L threitol; 1,5-pentanediol; 1,2-pentanediol; adonitol; xylitol; 1,2,6-hexanetriol; 1,2-octanediol; acemannan; mannitol; trehalose; galactrose; sorbitol; hexane; adonitol; butane; isopropyl; ethanol; methanol; or combinations thereof.

10. The bio-ink composition of claim 1, wherein the polypeptide comprises about 2% w/v to about 25% w/v of the ink and the humectant comprises about 2% w/v to about 30% w/v of the ink.

11. The bio-ink composition of claim 1, wherein the polypeptide comprises about 2% w/v to about 40% w/v of the ink.

12. The bio-ink composition of claim 1, wherein the humectant comprises about 2% w/v to about 30% w/v of the ink.

13. The bio-ink composition of claim 1, wherein a ratio of polypeptide:humectant is between about 5:1 and 2:1.

14. The bio-ink composition of claim 1, wherein the ink is further characterized in that a ratio of humectant:polypeptide at least in part modulates a degree of imparted crystallinity.

15. The bio-ink composition of claim 1, wherein the ink is further characterized in that a droplet size modulates a degree of imparted crystallinity.

16. The bio-ink composition of claim 16, wherein the droplet size is between about 0.1 nL and 30 nL.

17. The bio-ink composition of claim 1, wherein the ink is further characterized in that a thin printed layer can be removed from the substrate without breaking the layer.

18. The bio-ink composition of claim 1, further comprising a radiopaque marker.

19. The bio-ink composition of claim 18, wherein the radiopaque marker is or comprises iron or magnesium.

20. The bio-ink composition of claim 18, wherein the radiopaque marker is greater than about 10% w/v.

21. The bio-ink composition of claim 1, further comprising at least one agent.

22. The bio-ink composition of claim 21, wherein the at least one agent is selected from the group consisting of: anti-proliferative agents, antibodies or fragments or portions thereof (e.g., paratopes or complementarity-determining regions), antibiotics or antimicrobial compounds, antigens or epitopes, aptamers, biopolymers, carbohydrates, cell attachment mediators (such as RGD), cytokines, cytotoxic agents, diagnostic agents (e.g. contrast agents; radionuclides; and fluorescent, luminescent, and magnetic moieties), drugs, enzymes, growth factors or recombinant growth factors and fragments and variants thereof, hormone antagonists, hormones, immunological agents, lipids, metals, nanoparticles (e.g., gold nanoparticles), nucleic acid analogs, nucleic acids (e.g., DNA, RNA, siRNA, modRNA, RNAi, and microRNA agents), nucleotides, nutraceutical agents, oligonucleotides, peptide nucleic acids (PNA), peptides, prodrugs, prophylactic agents (e.g. vaccines), proteins, radioactive elements and compounds, small molecules, therapeutic agents (e.g. antibiotics, NSAIDs, glaucoma medications, angiogenesis inhibitors, neuroprotective agents), toxins, or any combinations thereof.

23. The bio-ink composition of claim 22, wherein the at least one agent is releasable.

24. The bio-ink composition of claim 23, wherein a controlled release of the at least one releasable agent is achieved by diffusion as the layers degrade, decompose, and/or delaminate.

25. A method of printing a bio-ink composition, the method comprising steps of:
flowing a bio-ink compositions from a print head onto a substrate;
moving the flowing ink and substrate relative to one another so that the ink is printed on the surface of the substrate.

26. The method of claim 25, wherein the step of flowing the ink from the print head further comprises:
applying a voltage to the ink as it exits the print head to cause the ink to form a Taylor cone; and
contacting a tip of the Taylor cone with the substrate.

27. The method of claim 26, wherein the step of applying a voltage comprises applying the voltage while dragging the Taylor cone across a surface of the substrate, thereby printing the ink on the surface of the substrate.

28. The method of claim 27, wherein the flow of the ink from the print head to the substrate is substantially continuous so that a non-interrupted printing of the ink forms along a path defined by movement.

29. The method of claim 28, wherein the surface of the substrate is irregular.

30. The method of claim 29, wherein the step of applying a voltage further comprises electrically controlling the applied voltage to selectably contact and disengage the Taylor cone from the surface.

31. The method of claim 26, further comprising printing at least one additional layer of the ink atop a printed layer, thereby printing a three-dimensional structure.

32. The method of claim 27, wherein the ink is or comprised of silk fibroin.

33. The method of claim 27, further comprising a step of rotating the substrate relative to the print head while dragging the Taylor cone across the surface of the substrate to form a tubular structure.

34. The method of claim 33, the step of rotating the substrate relative to the print head, wherein rotation is about an axis that is perpendicular to a direction of flow of the ink from the print head.

35. The method of claim 27, the step of applying the voltage, wherein the voltage is applied between a conductive extruder nozzle of the print head and a ground electrode on a side of the substrate opposite the print head.

36. A three-dimensional printer system, comprising:
a substrate having a printing surface;
a multi-motor stepper for precision movement;
a print head having at least one extruder configured to provide a biopolymer ink onto the printing surface;
a ground electrode; and
a power supply configured to apply a voltage between the at least one extruder nozzle and the ground electrode to cause the bio-ink composition to form a Taylor cone as it exits the extruder nozzle.

37. The printer of claim 36, further comprising a controller configured to control the applied voltage to selectably contact or disengage the Taylor cone from the surface.

38. The printer of claim 36, further comprising a programmed pattern so that the applied voltage is controlled to selectably contact or disengage the Taylor cone from the surface in a predetermined manner.

39. The printer of claim 36, wherein the print head has a plurality of extruders configured to dispense components of the ink during printing.

40. The printer of claim 36, wherein printed layers of the ink are characterized in that a droplet size modulates a degree of imparted crystallinity.

41. The printer of claim 40, wherein the droplet size is between about 0.1 nL and 30 nL.

42. The printer of claim 36, wherein the multi-motor stepper has a minimal increment of programmable linear movement is between about 0.05 μm and about 1.0 mm.

43. A surgical implant comprising:
a device body configured to be placed in situ in a patient; and
a biopolymer-ink pattern printed onto a surface of the device body.

44. The surgical implant of claim 43, wherein the ink comprises a radiopaque marker configured to be identifiable in situ via X-ray imaging.

45. The surgical implant of claim 44, wherein the radiopaque marker configured to be identifiable in situ via X-ray imaging indicates a presence of an agent.

46. The surgical implant of claim 44, wherein the ink comprises an agent.

47. The surgical implant of claim 46, wherein the agent is a releasable agent.

48. The surgical implant of claim 44, wherein the pattern printed onto a surface of the device body is configured to indicate a presence of an agent in the biopolymer ink.

49. The surgical implant of claim 44, wherein the pattern printed onto a surface of the device body is comprised of markings at respective ends of the device body to allow for identification of a location and/or position of the surgical implant via X-ray imaging during implantation.

50. The surgical implant of any claims 43-49, wherein the surgical implant is a stent.

51. The surgical implant of any claims 43-49, wherein the device body is tubing and the surgical implant is an anastomosis device.

52. The bio-ink composition of claim 1, wherein the ink comprises more than one part, a first part of the ink further comprises a protein gel and a second part of the ink further comprises a polysaccharide gel, for forming complex shapes, wherein the complex shapes are irregular and/or hollow.

53. The bio-ink composition of claim 52, the first part is a sacrificial support material ink and the second part is a permanent structural material ink, wherein the sacrificial support material ink is a blend of the polypeptide, the humectant, the solvent, and the protein gel and the permanent structural material ink is a blend of the polypeptide, the humectant, the solvent, and the polysaccharide gel.

54. The bio-ink composition of claim 53, wherein the sacrificial support material part is about 10% gelatin, about 5% silk, and about 1% glycerol, and the permanent structural material part is about 5% silk, about 5% agar, and about 1% glycerol.

55. The bio-ink composition of claim 54, when printed and combined with a media and heat, the support material dissolves leaving the permanent structural material having a desired shape.

56. The three-dimensional printer system of claim 36, wherein the at least one extruder comprises more than one extruder; and further comprises an aspirator when modulating ink blends.

57. The bio-ink composition of claim 1, wherein the polypeptide comprises a range of about 0.05 mM to about 10 mM of the ink and the humectant comprises a range of about 5 mM to about 1000 mM of the ink.

58. The bio-ink composition of claim 57, wherein the polypeptide comprises about 0.5 mM of the ink and the humectant comprises about 400 mM of the ink.
